(19) United States
(12) Patent Application Publication
(10) Pub. No.: US 2026/0201400 A1
(43) Pub. Date: Jul. 16, 2026

(54) RHIZOBIAL tRNA-DERIVED SMALL RNAs AND USES THEREOF FOR REGULATING PLANT NODULATION

(71) Applicant: Purdue Research Foundation, West Lafayette, IN (US)

(72) Inventors: Jianxin Ma, West Lafayette, IN (US); Bo Ren, Beijing (CN); Jingbo Duan, West Lafayette, IN (US); Xutong Wang, West Lafayette, IN (US)

(21) Appl. No.: 19/317,050

(22) Filed: Sep. 2, 2025

Related U.S. Application Data
(62) Division of application No. 17/611,802, filed on May 18, 2020, now Pat. No. 12,404,517.
(60) Provisional application No. 62/848,672, filed on May 16, 2019.

(51) Int. Cl. C12N 15/82

(52) U.S. Cl. CPC C12N 15/8262; C12N 15/8218

(57) ABSTRACT

Rhizobial infection is the key process for initiating symbiotic nitrogen-fixing root nodules in legumes, which requires specific recognition of signal molecules produced by the bacteria and their hosts. Here, it is established that rhizobial tRNA-derived small RNA fragments (tRFs) are crucial signal molecules modulating host nodulation, which uncovers a bacterial small RNA-mediated mechanism for prokaryote-eukaryote interaction. Transgenic plants are also provided that express a construct encoding rhizobial-derived tRNA, which is subsequently cleaved to produce artificial tRFs. Constructs and methods of producing the same are also provided, as well as modifications for repressing a mechanism for the negative regulation of nodulation present within plants.

### PRIORITY

This patent application is related to, claims the priority benefit of, and is divisional patent application of U.S. patent application Ser. No. 17/611,802, filed Nov. 16, 2021, which issues as U.S. Pat. No. 12,404,517 on Sep. 2, 2025, and which is related to, claims the priority benefit of, and is a 35 U.S.C. § 371 national stage application of International Patent Application No. PCT/US2020/033464 to Ma et al., filed May 18, 2020, which is related to and claims the priority benefit of U.S. Provisional Patent Application No. 62/848,672, filed May 16, 2019. The entire contents of each of the aforementioned priority applications are hereby expressly incorporated herein by reference in their entireties into this disclosure.

### STATEMENT OF GOVERNMENT SUPPORT

This invention was made with government support under 2015-67013-22811 and 2018-67013-27425, both awarded by the United States Department of Agriculture National Institute of Food and Agriculture (USDA-NIFA). The government has certain rights in the invention.

### FIELD

This disclosure is related to enhancing nodulation efficiency in legumes and for extending symbiotic nitrogen fixation capabilities to non-legumes. Particularly, Rhizobial tRNA-derived small RNA fragments (tRFs) and their impacts on soybean root nodule initiation provide alternative targets and mechanisms to increase nitrogen fixation capability in plants.

### BACKGROUND

This section introduces aspects that may help facilitate a better understanding of the disclosure. Accordingly, these statements are to be read in this light and are not to be understood as admissions about what is or is not prior art.

Legumes are plants, such as soybeans, alfalfa, clover, peas, beans, lentils, lupins, mesquite, and peanuts, that form a symbiotic relationship between their roots and bacteria, specifically of the family Rhizobiaceae. Rhizobial lipo-chitooligosaccharidic nodulation (Nod) factors are the key signal molecules responsible for induction of plant responses that lead to nodule formulation. Upon perception of plant flavonoids, Rhizobia secrete Nod factors, which induce root hair curling around the bacteria and the subsequent development of infection treads that allow the bacteria to penetrate the cortical cells of the roots to form nodules.

Nodulation in legumes provides a major conduit of available nitrogen into the biosphere. More specifically, the plant provides the bacteria both sustenance and an energy source in the form of adenosine triphosphate (ATP) that is generated by photosynthesis. In return, the bacteria fix elemental nitrogen from the atmosphere into ammonia, a usable form of nitrogen that is digestible by plants. This interaction, called symbiotic nitrogen fixation (SNF), is important because it enables the plant hosts to access atmospheric nitrogen and, thus, provides a rich nitrogen source to the plant. SNF occurs in specialized root organs known as nodules of legumes and is a major source of nitrogen in agricultural and natural eco-systems. As nitrogen is the nutrient that most frequently limits the growth of green plants, optimizing its application is a key to optimizing plant yield.

Legumes are generally higher in protein content than other plant families due to the availability of nitrogen from nitrogen fixation. The high protein content makes legumes one of the most important food crops for both human consumption and animal feed. Further, legumes are used in crop rotation practice to increase the nitrogen content of soils, through SNF, for future growth seasons and to reduce the amount of fertilizer required. This has cost benefits to the grower and can reduce nitrogen runoff.

To optimize legume crop yield, it is important to increase efficiencies of nodulation and nitrogen fixation, which maintain adequate nitrogen levels in the plants. Accordingly, what is needed is an efficient and cost-effective approach to improve nodulation of legumes and, thus, increase the plant's yield potential.

### BRIEF SUMMARY

Transgenic leguminous plants, plant roots, and plant cells are provided. In at least one exemplary embodiment, such leguminous plants, plant roots, and plant cells comprise at least one mutation to express a polynucleotide construct encoding rhizobial-derived RNA having a hairpin structure that is cleaved in vivo in the plant root into an artificial small RNA fragment (i.e. “artificial tRFs”). The artificial small RNA fragments/tRFs downregulate or repress one or more target genes of the leguminous plant, plant root or plant cell that are negative regulators of nodulation. In this manner, the transgenic leguminous plant, plant root, or plant cell produces increased nodules when inoculated with a rhizobium as compared to nodules produced from a corresponding inoculated wild type legume plant, plant root, or plant cell. In certain embodiments, the leguminous plant, plant root, or plant cell is Glycine max and the artificial small RNA fragment has a nucleotide sequence selected from a group consisting of SEQ ID NOs: 1-3, SEQ ID NOs: 50-70, and a substantially homologous nucleic acid sequence of SEQ ID NOs: 1-3, SEQ ID NOs: 50-70. In at least one exemplary embodiment, the nucleotide sequence of the artificial small RNA fragment is SEQ ID NO: 1, SEQ ID NO: 2, SEQ ID NO: 3, or a substantially homologous nucleic acid sequence of SEQ ID NOs: 1-3. In an alternative embodiment, the leguminous plant, plant root, or plant cell is Rhizobium etli and the nucleotide sequence of the artificial small RNA fragment is SEQ ID NOs: 34-49 or a substantially homologous nucleic acid sequence of SEQ ID NOs: 34-49.

In other embodiments, the least one mutation may further comprise a second mutation in at least one of the one or more target genes of the leguminous plant, plant root or plant cell. There, the second mutation can be designed to downregulate or repress the one or more target genes. Such target genes may comprise any host plant genes that are negative regulators of the nodulation mechanism such as, by way of nonlimiting example, GmRHD3a, GmRHD3b, GmHAM4a, GmHAM4b, GmLRX5, or an ortholog of the any of the foregoing. It will further be appreciated that one or any combination of the foregoing genes may also comprise this second mutation. For example, the second mutation may be at both GmRHD3a and GmRHD3b, or both GmHAM4a and GmHAM4b. Additionally or alternatively, the second mutation may comprise a deletion. There, all of the target genes may be deleted or, one or more of the target genes may be deleted while other target genes are simply repressed or unmodified.

Methods for increasing nodulation activity in a leguminous plant root are also provided. Such methods may comprise repressing or downregulating expression of one or more target genes of a host plant root or cell, each of the target genes being a negative regulator of nodulation in the host, and inoculating, or having inoculated, the modified plant host root or cell with at least one rhizobium to initiate nodulation. In at least one embodiment, the target gene(s) may be one or more of: RHD3, HAM4, LRX5, an ortholog of RHD3, an ortholog of HAM4, and an ortholog of LRX5 and/or the species or strain(s) of the at least one rhizobium is selected from a group consisting of: Bradyrhizobium japonicum, Rhizobium etli, Sinorhizobium meliloti, Rhizobium leguminosarum, Parasponia rhizobium, and Mesorhizobium loti. In at least one exemplary embodiment, the host root or cell is Glycine max and each target gene is selected from a group consisting of: GmRHD3a, GmRHD3b, GmHAM4a, GmHAM4b, and GmLRX5.

The repressing or downregulating step may comprise introducing and expressing a first polynucleotide construct encoding rhizobial-derived RNA, the rhizobial-derived RNA for producing one or more artificial small RNA fragments (“artificial tRFs”) in the plant root that downregulate at least one of the target genes. In at least one embodiment, such rhizobial-derived RNA may have a hairpin structure that is cleaved in vivo to produce the artificial tRFs in the plant root. Furthermore, each of the artificial tRFs may have a nucleotide sequence selected from a group consisting of: SEQ ID NOs: 1-3, SEQ ID NOs: 34-70, or a substantially homologous nucleic acid sequence of SEQ ID NOs: 1-3 or SEQ ID NOs: 34-70. Still further, each of the artificial tRFs may have a nucleotide sequence of SEQ ID NO. 1, SEQ ID NO. 2, SEQ ID NO. 3, or a substantially homologous nucleic acid sequence of SEQ ID NO. 1, SEQ ID NO. 2, or SEQ ID NO. 3. Additionally or alternatively, the repressing or downregulating step may comprise constructing a CRISPR-Cas9 vector to silence one or more of the target genes and expressing the vector in the host plant root or cell. In at least one exemplary embodiment, the host root or cell is Glycine max and each target gene is selected from a group consisting of: GmRHD3a (which encodes SEQ ID NO: 80), GmRHD3b (which encodes SEQ ID NO: 81), GmHAM4a (which encodes SEQ ID NO: 82), GmHAM4b (which encodes SEQ ID NO: 83), GmLRX5 (which encodes SEQ ID NO: 84).

In yet another embodiment, where the target gene(s) are GmRHD3a, GmRHD3b, or both, the CRISPR-Cas9 vector comprises SEQ ID NOs. 74 and 75; where the target gene(s) are GmHAM4a, GmHAM4b, or both, the CRISPR-Cas9 vector comprises SEQ ID NOs. 76 and 77; and/or where at least one of the target genes is GmLRX5, the CRISPR-Cas9 vector comprises SEQ ID NOs. 78 and 79. In at least one exemplary embodiment, the repressing or downregulating step comprises: introducing and expressing a first polynucleotide construct encoding rhizobial-derived RNA that is cleaved in vivo to produce one or more artificial small RNA fragments in the plant root; and expressing a CRISPR-Cas9 vector in the host plant root or cell, the CRRISPR-Cas9 vector constructed to silence one or more of the following host genes: GmRHD3a (which encodes SEQ ID NO: 80), GmRHD3b (which encodes SEQ ID NO: 81), GmRHD3a/GmRHD3b, GmHAM4a (which encodes SEQ ID NO: 82), GmHAM4b (which encodes SEQ ID NO: 83), GmHAM4a/GmHAM4b, GmLRX5 (which encodes SEQ ID NO: 84).

Additional embodiments of the method provide for repressing or downregulating the expression of the one or more host genes by introducing and expressing a second polynucleotide construct into the host plant root or cell, where the second polynucleotide construct selectively represses expression of the one or more target genes. Certain methods of the present disclosure may additionally or alternatively include the at least one rhizobium comprising one or more species or strains of a rhizobial microorganism that expresses a third polynucleotide construct that enhances production of one or more transfer ribonucleic acid-derived fragments (tRFs) as compared to production of tRFs in a corresponding wild-type rhizobial microorganism.

Novel constructs are also provided in the present disclosure, with embodiments of such constructs encoding a ribonucleic acid transcript that folds into a hairpin structure and is cleaved in vivo into an artificial small ribonucleic acid molecule that, when expressed in a leguminous plant root, increases root nodule formation. In at least one non-limiting embodiment, the artificial small ribonucleic acid molecule has a nucleotide sequence selected from a group consisting of SEQ ID NOs. 1-3, 34-49, and 50-70. Such novel constructs may be utilized in any of the transgenic plants, plant roots, plant cells, and methods of the present disclosure.

Still further, certain embodiments provide for a rhizobial microorganism exhibiting selectively amplified production of one or more transfer ribonucleic acid-derived fragments (tRFs) as compared to production of tRFs in a corresponding wild-type rhizobial microorganism. For example, such a rhizobial microorganism may express a polynucleotide construct that enhances production of one or more tRFs and, in at least one embodiment, such tRFs may be capable of repressing or downregulating expression of at least one target gene of a host organism (such as the target genes previously described). In at least one embodiment, the at least one target genes of the host organism is selected from a group consisting of: RHD3, HAM4, LRX5 or an ortholog thereof. In yet another embodiment, the host organism is Glycine max and each of the at least one target genes is selected from a group consisting of: GmRHD3a (which encodes SEQ ID NO: 80), GmRHD3b (which encodes SEQ ID NO: 81), GmHAM4a (which encodes SEQ ID NO: 82), GmHAM4b (which encodes SEQ ID NO: 83), and GmLRX5 (which encodes SEQ ID NO: 84). Certain other embodiments provide for a compound comprising one or more species or strains of the rhizobial microorganism described herein, wherein the tRFs repress or downregulate expression of at least one target gene of a host plant root and the compound is formulated in an inoculant composition.

### BRIEF DESCRIPTION OF THE SEQUENCE LISTINGS

SEQ ID NO: 1 is a nucleic acid sequence of a Bradyrhizobium japonicum-derived tRF:
CGAUCCCUUGUGCGCCCACCA;
SEQ ID NO: 2 is a nucleic acid sequence of a B. japonicum-derived tRF:
CGAUUCCCUUCACCCGCUCCA;
SEQ ID NO: 3 is a nucleic acid sequence of a B. japonicum-derived tRF:
UGGGGAAUGGUGUAACGGUAG;
SEQ ID NO: 4 is an artificial nucleic acid sequence of oligonucleotides targeting RHD3
(RHD3_pSC_F): ATTGAGGTGAAGTTGGCTGAATG;
SEQ ID NO: 5 is an artificial nucleic acid sequence of oligonucleotides targeting RHD3
(RHD3_pSC_B): AACCATTCAGCCAACTTCACCTC;
SEQ ID NO: 6 is an artificial nucleic acid sequence of oligonucleotides targeting HAM4
(HAM4_pSC_F): ATTGCCTGTGGGGATGAGGGAAC;
SEQ ID NO: 7 is an artificial nucleic acid sequence of oligonucleotides targeting HAM4
(HAM4_pSC_B): AACGTTCCCTCATCCCCACAGGC;
SEQ ID NO: 8 is an artificial nucleic acid sequence of oligonucleotides targeting LRX5
(LRX5_pSC_F): ATTGTTGAAACTGCTCTACGAAC;
SEQ ID NO: 9 is an artificial nucleic acid sequence of oligonucleotides targeting LRX5
(LRX5_pSC_B): AACGTTCGTAGAGCAGTTTCAAC;
SEQ ID NO: 10 is a nucleic acid sequence of a wild-type B. japonicum-derived tRF:
CAAATCCTATCCCCGCAACCA;
SEQ ID NO: 11 is a nucleic acid sequence of a wild-type B. japonicum-derived tRF:
CAAATCCTCTCGCTCCGACCA;
SEQ ID NO: 12 is a nucleic acid sequence of a wild-type B. japonicum-derived tRF:
CAATCCTGTCTGGCAGCACCA;
SEQ ID NO: 13 is a nucleic acid sequence of a wild-type B. japonicum-derived tRF:
CGAATCCAGCTGCCCCGACCA;
SEQ ID NO: 14 is a nucleic acid sequence of a wild-type B. japonicum-derived tRF:
CGAATCCAGGTTCCCCAGCCA;
SEQ ID NO: 15 is a nucleic acid sequence of a wild-type B. japonicum-derived tRF:
CGAATCCCACTCTCTCCGCCA;
SEQ ID NO: 16 is a nucleic acid sequence of a wild-type B. japonicum-derived tRF:
CGAATCCCTCCCTCTCCGCCA;
SEQ ID NO: 17 is a nucleic acid sequence of a wild-type B. japonicum-derived tRF:
CGAATCCTGCCACTCCGACCA;
SEQ ID NO: 18 is a nucleic acid sequence of a wild-type B. japonicum-derived tRF:
CGAATCGTGTCGGGTGCGCCA;
SEQ ID NO: 19 is a nucleic acid sequence of a wild-type B. japonicum-derived tRF:
CGAGCCCACCCAACTGTACCA;
SEQ ID NO: 20 is a nucleic acid sequence of a wild-type B. japonicum-derived tRF:
CGAGCCCTGCCACTCCTGCCA;
SEQ ID NO: 21 is a nucleic acid sequence of a wild-type B. japonicum-derived tRF:
CGAGTCATCCCGGGATCGCCA;
SEQ ID NO: 22 is a nucleic acid sequence of a wild-type B. japonicum-derived tRF:
CGAGTCCCTCCGAGCGCACCA;
SEQ ID NO: 23 is a nucleic acid sequence of a wild-type B. japonicum-derived tRF:
CGATCCCGTCTGGCTCCACCA;
SEQ ID NO: 24 is a nucleic acid sequence of a wild-type B. japonicum-derived tRF:
CGATCCCTGTCGTGCCCACCA;
SEQ ID NO: 25 is a nucleic acid sequence of a wild-type B. japonicum-derived tRF:
CGATCCCTTGTGCGCCCACCA;
SEQ ID NO: 26 is a nucleic acid sequence of a wild-type B. japonicum-derived tRF:
CGATTCCCTTCACCCGCTCCA;
SEQ ID NO: 27 is a nucleic acid sequence of a wild-type B. japonicum-derived tRF:
CGATTCCCTTCGCCCGCTCCA;
SEQ ID NO: 28 is a nucleic acid sequence of a wild-type B. japonicum-derived tRF:
CGATTCCGCCCCTGGGCACCA;
SEQ ID NO: 29 is a nucleic acid sequence of a wild-type B. japonicum-derived tRF:
GCGCTCGTGGCGGAACTGGTA;
SEQ ID NO: 30 is a nucleic acid sequence of a wild-type B. japonicum-derived tRF:
GCGGGTGTAGCTCAGTGGTAG;
SEQ ID NO: 31 is a nucleic acid sequence of a wild-type B. japonicum-derived tRF:
TCCTCGGTAGCTCAGCGGTAG;
SEQ ID NO: 32 is a nucleic acid sequence of a wild-type B. japonicum-derived tRF:
TGGGGAATGGTGTAACGGTAG;
SEQ ID NO: 33 is a nucleic acid sequence of a wild-type B. japonicum-derived tRF:
TTGGTAGACGCAAGGGACTTA;
SEQ ID NO: 34 is a nucleic acid sequence of a wild-type Rhizobium etli-derived tRF:
CAAATCCCTCCTCCGCTACCA;
SEQ ID NO: 35 is a nucleic acid sequence of a wild-type Rhizobium etli-derived tRF:
CAAGTCTTCCCGGGCCCACCA;
SEQ ID NO: 36 is a nucleic acid sequence of a wild-type Rhizobium etli-derived tRF:
CGAGTCCAGGTTCCCCAGCCA;
SEQ ID NO: 37 is a nucleic acid sequence of a wild-type Rhizobium etli-derived tRF:
AGTAGGTCAGAGCAGAGGAAT;
SEQ ID NO: 38 is a nucleic acid sequence of a wild-type Rhizobium etli-derived tRF:
AGTAGGTCAGAGCAGAGGAAT;
SEQ ID NO: 39 is a nucleic acid sequence of a wild-type Rhizobium etli-derived tRF:
AGTAGGTCAGAGCAGAGGAAT;
SEQ ID NO: 40 is a nucleic acid sequence of a wild-type Rhizobium etli-derived tRF:
CAAGTCTTCCCGGGCCCACCA;
SEQ ID NO: 41 is a nucleic acid sequence of a wild-type Rhizobium etli-derived tRF:
CGAATCACTCCACTCCGACCA;
SEQ ID NO: 42 is a nucleic acid sequence of a wild-type Rhizobium etli-derived tRF:
CGAATCCGGCCCGGGGAGCCA;
SEQ ID NO: 43 is a nucleic acid sequence of a wild-type Rhizobium etli-derived tRF:
CGAGCCCTGCTGCCCCTGCCA;
SEQ ID NO: 44 is a nucleic acid sequence of a wild-type Rhizobium etli-derived tRF:
CGAGTCTCTCATCGCCCACCA;
SEQ ID NO: 45 is a nucleic acid sequence of a wild-type Rhizobium etli-derived tRF:
GATTTTCATTCTGTAAAGAGG;
SEQ ID NO: 46 is a nucleic acid sequence of a wild-type Rhizobium etli-derived tRF:
GATTTTCATTCTGTAAAGAGG;
SEQ ID NO: 47 is a nucleic acid sequence of a wild-type Rhizobium etli-derived tRF:
GTAGGTCAGAGCAGAGGAATC;
SEQ ID NO: 48 is a nucleic acid sequence of a wild-type Rhizobium etli-derived tRF:
GTAGGTCAGAGCAGAGGAATC;
SEQ ID NO: 49 is a nucleic acid sequence of a wild-type Rhizobium etli-derived tRF:
GTAGGTCAGAGCAGAGGAATC;
SEQ ID NO: 50 is a nucleic acid sequence of a B. japonicum-derived tRF:
CAAAUCCUAUCCCCGCAACCA;
SEQ ID NO: 51 is a nucleic acid sequence of a B. japonicum-derived tRF:
CAAAUCCUCUCGCUCCGACCA;
SEQ ID NO: 52 is a nucleic acid sequence of a B. japonicum-derived tRF:
CAAUCCUGUCUGGCAGCACCA;
SEQ ID NO: 53 is a nucleic acid sequence of a B. japonicum-derived tRF:
CGAAUCCAGCUGCCCCGACCA;
SEQ ID NO: 54 is a nucleic acid sequence of a B. japonicum-derived tRF:
CGAAUCCAGGUUCCCCAGCCA;
SEQ ID NO: 55 is a nucleic acid sequence of a B. japonicum-derived tRF:
CGAAUCCCACUCUCUCCGCCA;
SEQ ID NO: 56 is a nucleic acid sequence of a B. japonicum-derived tRF:
CGAAUCCCUCCCUCUCCGCCA
SEQ ID NO: 57 is a nucleic acid sequence of a B. japonicum-derived tRF:
CGAAUCCUGCCACUCCGACCA;
SEQ ID NO: 58 is a nucleic acid sequence of a B. japonicum-derived tRF:
CGAAUCGUGUCGGGUGCGCCA;
SEQ ID NO: 59 is a nucleic acid sequence of a B. japonicum-derived tRF:
CGAGCCCACCCAACUGUACCA;
SEQ ID NO: 60 is a nucleic acid sequence of a B. japonicum-derived tRF:
CGAGCCCUGCCACUCCUGCCA;
SEQ ID NO: 61 is a nucleic acid sequence of a B. japonicum-derived tRF:
CGAGUCAUCCCGGGAUCGCCA;
SEQ ID NO: 62 is a nucleic acid sequence of a B. japonicum-derived tRF:
CGAGUCCCUCCGAGCGCACCA;
SEQ ID NO: 63 is a nucleic acid sequence of a B. japonicum-derived tRF:
CGAUCCCGUCUGGCUCCACCA;
SEQ ID NO: 64 is a nucleic acid sequence of a B. japonicum-derived tRF:
CGAUCCCUGUCGUGCCCACCA;
SEQ ID NO: 65 is a nucleic acid sequence of a B. japonicum-derived tRF:
CGAUUCCCUUCGCCCGCUCCA;
SEQ ID NO: 66 is a nucleic acid sequence of a B. japonicum-derived tRF:
CGAUUCCGCCCCUGGGCACCA;
SEQ ID NO: 67 is a nucleic acid sequence of a B. japonicum-derived tRF:
GCGCUCGUGGCGGAACUGGUA;
SEQ ID NO: 68 is a nucleic acid sequence of a B. japonicum-derived tRF:
GCGGGUGUAGCUCAGUGGUAG;
SEQ ID NO: 69 is a nucleic acid sequence of a B. japonicum-derived tRF:
UCCUCGGUAGCUCAGCGGUAG;
SEQ ID NO: 70 is a nucleic acid sequence of a B. japonicum-derived tRF:
UUGGUAGACGCAAGGGACUUA;
SEQ ID NO: 71 is a wild-type B. japonicum tRNA:
GGGCGCAUAGCUCAGCGGGAGAGCGUUCCCUUCACACGGGAGAGGUCCAAGGUUCGAUCCCUUGUGCGCCCACCA;
SEQ ID NO: 72 is a wild-type B. japonicum tRNA:
GCGGGUGUAGCUCAAUGGUAGAGCAGCAGCCUUCCAAGCUGAAUACGAGGGUUCGAUUCCCUUCACCCGCUCCA;
SEQ ID NO: 73 is a wild-type B. japonicum tRNA:
UGGGGAAUGGUGUAACGGUAGCACAACAGACUCUGACUCUGUUUGUCUUGGUUCGAAUCCAGGUUCCCCAGCCA;
SEQ ID NO: 74 is an artificial nucleic acid sequence of a primer for an overexpression
construct RHD3b (RHD3b_CDS_F): GCGCGTCGACATGGCAAATAGTGAGACTTGTTG;
SEQ ID NO: 75 is an artificial nucleic acid sequence of a primer for an overexpression
construct RHD3b (RHD3b_CDS_B): gcgcTCTAGACTACTCATCTTTTAATGGACTTGC;
SEQ ID NO: 76 is an artificial nucleic acid sequence of a primer for an overexpression
construct HAM4a (HAM4a_CDS_F): TTGCctcgagATGAGAGTTCCCGTTCCCTCATCC;
SEQ ID NO: 77 is an artificial nucleic acid sequence of a primer for an overexpression
construct HAM4a (HAM4a_CDS_B): GCTTggtaccCTAACAGCGCCATGCTGACGTGGCA;
SEQ ID NO: 78 is an artificial nucleic acid sequence of a primer for an overexpression
construct LRX5 (LRX5_CDS_F): gcgcGTCGACATGATGATGATGATGAAGAAGAAGG;
SEQ ID NO: 79 is an artificial nucleic acid sequence of a primer for an overexpression
construct LRX5 (LRX5_CDS_B): gcaaTCTAGACTAATAAAAGGGTGGTGGTGGAGGC.

SEQ ID NO: 80 is an amino acid sequence encoded by Glycine max gene GmRHD3a (Glyma.07G010200).

SEQ ID NO: 81 is an amino acid sequence encoded by Glycine max gene GmRHD3b (Glyma.08G0193200).

SEQ ID NO: 82 is an amino acid sequence encoded by Glycine max gene GmHAM4a (Glyma.01G177200).

SEQ ID NO: 83 is an amino acid sequence encoded by Glycine max gene GmHAM4b (Glyma.11G065200).

SEQ ID NO: 84 is an amino acid sequence encoded by Glycine max gene GmLRX5 (Glyma.08G039400).

In addition to the foregoing, the above-described sequences have been submitted electronically in computer readable form, XML format (file entitled “68447-06SequenceListing.xml; file size=81,605 bytes; date created Mar. 30, 2026), which is hereby incorporated by reference in its entirety. The information recorded in computer readable form is identical to the written Sequence Listings provided above, pursuant to 37 C.F.R. § 1.821(f).

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIG. 1 is a schematic depiction of the mechanisms that regulate soybean nodulation and, thus, nodule numbers, with the lines labeled X indicating plant-based negative regulation of nodulation and lines labeled Y indicating Rhizobium-based positive regulation of nodulation as described herein (AON=autoregulation of nodulation; NON=nitrogen regulation of nodulation; and RIC1 and RIC2=nitrogen-suppression CLAVATA3/EMBRYO SURROUNDING REGION-RELATED (CLE) peptides 1 and 2);

FIG. 2 illustrates graphical data relating to the sizes, positions, and abundance of rhizobial tRFs in Rhizobia and root nodules, with subparts A-D representing data on size distribution and relative abundance of rhizobial tRFs in free-living Rhizobia (strain USDA 110) (●), and soybean root nodules induced by USDA 110 (▪ and ▴), subparts E-H representing data on the positions and abundance of 21-nt tRFs along rhizobial tRNAs in soybean root nodules induced by USDA 110, with the frequencies reflecting the relative coverage of each site along individual tRNAs by 21-nt tRFs, and subparts I-K illustrating the structures of three wild-type rhizobial tRNAs arranged in a hairpin structure and the physical positions of three tRFs as indicated between the black lines (Bj-tRF001/SEQ ID NO: 1, Bj-tRF002/SEQ ID NO: 2, and Bj-tRF003/SEQ ID NO: 3, respectively) produced by corresponding tRNAs, with subpart I having SEQ ID NO: 71, subpart J having SEQ ID NO: 72, and subpart K having SEQ ID NO: 73;

FIG. 3A is a neighbor-joining tree representation of the phylogenic relationship of tRNAs in the Bradyrhizobium japonicum genome strain USDA110k prepared based on the tRNA sequences (each named in a format of “Bj (Abbreviation of the rhizobium species)”-“amino acid transferred by a particular tRNA copy”-“order of tRNA transferring a same amino acid by recognizing multiple codons”-“(condo)”), with the numbers adjacent to nodes indicating the bootstrap values from the test of 1000 replicates;

FIGS. 3B and 3C illustrate graphical data relating to the size distribution and abundance of rhizobial tRFs in Rhizobia and root nodules, with the x-axis indicating the size of major rhizobial tRFs that range from 18-24nt and they-axis indicating the relative abundance of the tRFs of different sizes; lower plot line in all cases represents size distribution and relative abundance of rhizobial tRFs in free-living rhizobia (strain USDA110), whereas the plot lines labeled A and B represent size distribution and relative abundance of rhizobial tRFs in soybean 10-dpi nodules and 20-dpi nodules, respectively;

FIGS. 4A and 4B show data related to the physical positions and abundance of 21-nt tRFs along rhizobial tRNAs in soybean root nodules where the 10-dpi and 20-dpi nodules were induced by USDA 110 and the frequencies shown reflect the relative coverage of each site along individual tRNAs by the 21-nt tRFs, with each x-axis representing nucleotide locations of a tRNA in B. japonicum USDA110 and each y-axis indicating the frequencies of individual nucleotides along the tRNA that are covered by the 21-nt tRFs derived from the tRNA in the 10-dpi or 20-dpi nodules induced by USDA110;

FIG. 5A shows graphical data illustrating the abundance of the three tRFs, measured by means of stem-loop quantitative RT-PCR, in free-living B. japonicum (B.j.) USDA 110 (1) and 10-day and 20-day post-inoculation (dpi) nodules (2 and 3, respectively), with values shown as means±SE from three biological replicates (one set as “1” and the others adjusted accordingly), and asterisks indicating the significance level at p<0.01 (Student's t test);

FIG. 5B shows graphical data illustrating the expression of the identified tRF target genes, measured with quantitative RT-PCR in the 10-day-old and 20-day-old nodules (2 and 4, respectively) and uninoculated soybean roots (1 and 3), with values shown as means±SE from three biological replicates (one set as “1” and the others adjusted accordingly), and asterisks indicating the significance level at p<0.01 (Student's t test);

FIG. 5C shows a gel following the enrichment of three rhizobial tRFs in soybean root nodules, with abundance shown relative to that of the transcripts from a B. japonicum gene b11631 in free living B. japonicum strain USDA110, and 10-dpi and 20-dpi nodules detected by stem-loop RT-PCR using the reaction from the 20-dpi RNA sample without adding reverse transcriptase as a negative control; the specificity of stem-loop RT-PCR products from individual tRFs confirmed by sequencing of the amplified fragments and equal amounts of total RNAs from different samples loaded on the gel to visualize different sizes and relative abundances of rhizobial rRNAs and soybean rRNAs;

FIGS. 6A-6C show the three identified tRFs of the present disclosure, their putative target transcripts, and the cleavage sites and frequencies (indicated with arrows and ratios) that were detected in the 20-dpi nodules;

FIG. 6D shows the sequences of artificial small RNAs, asRNA001 (SEQ ID NO: 1) and asRNA001*, and a BJ-tRF001 target site as identified herein, and FIG. 6E shows graphical data illustrating that target host gene GmRHD3a 3b has reduced expression in soybean asR001-transgenic root protoplasts as compared with that in asR001*-transgenic root protoplasts 20 h post transformation, with levels of gene expression relative to that of GmCos4 as detected by qRT-PCR and shown as means±standard errors from three biological replicates (in each panel, the expression level of one sample was set at 1 and those of the others adjusted accordingly; * and ** indicate significant difference at P<0.05 and P<0.01, respectively (t-test);

FIG. 6F shows the cleavage sites within mRNAs of identified rhizobial tRF target genes pursuant to the present disclosure, with distribution (x-axes) of cleavage sites within the mRNAs of the five soybean genes and times (y-axes) of cleavage in 20-dpi nodules and uninoculated roots detected by sequencing the RLM-RACE products; dots pointing to the sites between the boxed nucleotides represent cleavage sites, some of the 5′ and 3′UTRs (labeled U), introns (labeled I), and exons (labeled E) of the soybean genes, their mRNAs, and the identified tRF target sites indicated by their respective labels, alignments and arrows;

FIG. 7A shows the sequences of hairy root mutations created by site-specific CRISPR-Cas9 editing of GmRHD3a 3b, GmHAM4a 4b, and GmLRX5, with the wild-type of exonic sequence of each gene for editing and the corresponding amino acid sequence shown on the top of each panel, the protospacer-adjacent motifs (PAMs) used for design guided RNAs underlined, and nucleotide deletions, insertions and substitutions created by CRISPR-Cas9 each positioned within a box (□); asterisks denote the consensus nucleotides;

FIG. 7B, subparts A-C show graphical depictions of the rhizobial tRF targets and the CRISPR-Cas9-edited forms of the targets from FIG. 7A in the transgenic roots, with the expression levels of the wild-type (WT) tRF targets and CRISPR-Cas9-edited forms of the targets shown relative to that of GmELF1b detected by qRT-PCR and shown as means±s.e. from three technical replicates, the transgenic root samples being the same as described in FIG. 8A, which were collected 28 dpi, controls being transgenic roots of empty vectors used for developing the CRISPR-Cas9 knockouts, and, in each panel, the expression level of one sample set as 1, and those of the others adjusted accordingly (“−1” and “−2” indicate two hairy roots with the same gene or a pair of duplicated genes edited by CRISR-Cas9 using a same guide RNA, each with three technical replicates;

FIG. 8A shows photographs of the roots of mutants for each of the five genes (GmRHD3a, GmRHD3b, GmHAM4a, GmHAM4b, and GmLRX5) and both copies of the two duplicated gene pairs (GmRHD3a/GmRHD3b and GmHAM4a/GmHAM4b), with each of the knockouts having increased nodule numbers over the controls;

FIG. 8B shows a graph of nodule numbers from the mutants and controls as exemplified in FIG. 8A, with all data points represented by dots and shown as box and whisker plots displaying 95 to 5% interval from three biological replicates (12 plants per replicate) collected 28 days after inoculation (asterisks indicate the significance level of P<0.01 (Student's t test); shown as means standard deviations from three independent experiments (n=12 per experiment);

FIG. 9A shows photographs of transgenic roots engineered to overexpress three of the tRF targets of the present disclosure (GmRHD3b, GmHAM4a, and GmLRX5), with all mutants producing reduced nodule numbers relative to the control; FIG. 9B shows a graph of the nodule numbers from the mutants and control as exemplified in FIG. 9A (all data points represented by dots and shown as box and whisker plots displaying 95 to 5% interval from three biological replicates (12 plants per replicate) collected 28 days after inoculation (asterisks indicate the significance level of P<0.01 (Student's t-test); shown as means±standard deviations from three independent experiments (n=12 per experiment)); and FIG. 9C shows graphical data related to the overexpression of rhizobial tRF targets in hairy roots as compared with that in empty vector transgenic controls, with ** indicating significant difference (t-test, P<0.01), levels of gene expression relative to that of GmELF1b as detected by qRT-PCR, shown as means±errors from three biological replicates, and, in each panel, the expression level of one sample was set as 1 and those of the other adjusted accordingly;

FIG. 10A shows graphical data related to the increased expression of the rhizobial tRF targets in the STTM hairy roots that inhibited the rhizobial tRF function (S) as compared with that in an empty vector transgenic control (C), with levels of gene expression relative to that of GmELF1b as detected by qRT-PCR, shown as means±s.e. from three biological replicates, and all samples collected 28 dpi; FIG. 10B shows photographs of transgenic roots engineered to silence individual tRFs by means of STTM; FIG. 10C shows a graph of the nodule numbers of the roots shown in FIG. 10A all data points represented by dots and shown as box and whisker plots displaying 95 to 5% interval from three biological replicates (12 plants per replicate) collected 28 days after inoculation, and shown as means±standard deviations from three independent experiments (n=12 per experiment)); and FIG. 10D shows graphical data related to rhizobia tRFs and the expression of the identified target genes in STTM transgenic roots and nodules, where the expression levels of the tRFs and soybean genes relative to that of GmELF1b were detected by qRT-PCR, shown as means±s.e. from three biological replicates, controls were transgenic roots of empty-vector used for developing the STTM tRF-silencing roots, and all samples collected 28 dpi (FIGS. 10A, 10C and 10D, asterisks in indicate the significance level of P<0.01 (Student's t test) and, in each panel of FIGS. 10A and 10D, the expression level of one sample was set as 1 and those of the other adjusted accordingly);

FIGS. 11A and 11B show the rhizobial tRF abundance and expression of their target genes at early-stage rhizobial infection of soybean root hairs, with FIG. 11A showing graphical data representative of the enrichment of rhizobial tRFs relative to the transcripts of rhizobium b11631 in root hairs (unmarked) as compared to that in free-living rhizobia (C) detected by stem-loop RT-qRT-PCR, and FIG. 11B showing graphical data showing reduced expression of the rhizobial tRF targets relative to GmELF1b in root hairs as compared with expression in uninoculated root hairs measured by qRT-PCR (values in FIGS. 11A and 11B shown as means±s.e. from three biological replicates, and the value of one sample was set as 1 and those of the others adjusted accordingly; asterisks indicating significant difference at P<0.05 (t-test);

FIG. 12 shows graphical data related to root hair numbers and lengths in overexpression and STTM transgenic hairy roots, with subpart A showing a comparison between soybean hairy roots overexpressing the rhizobial tRF target genes and hairy roots transformed with the empty vector at 6 dpi, and subpart B showing a comparison between soybean STTM hairy roots inhibiting the rhizobial tRF function and hairy roots transformed with the empty vector at 6 dpi (values shown as means s.d. of the means from three independent experiments (each experiment, n=25);

FIG. 13 shows data related to modulation of early-stage rhizobial infection by rhizobial tRFs and their targets in soybean, with subparts A and B showing the morphological differences between the root hairs overexpressing the rhizobial tRF targets and the negative control (subpart A) and between the STTM root hairs inhibiting the rhizobial tRF function and the negative control (subpart B) and subpart C showing graphical data related to the quantitation of deformed root hairs and curled root hairs with infection foci in samples as exemplified in subparts A and B (values shown as means±SD from three biological replicates (n=25 hairy roots per replicate) and asterisks indicating the significant level at P<0.05 (Student's t test);

FIGS. 14A-14C show graphical data related to rhizobial tRF-guided gene regulation by hijacking the host RNAi machinery, with FIG. 14A illustrating data related to the abundance of artificial miRNAs measured with stem-loop qRT-PCR in aMIR-tRF001 (2) and aMIR-tRF003 (3) transgenic roots and respective empty-vector transgenic roots (1) at 28 dpi, FIG. 14B showing expression of the identified tRF/artificial miRNA targets measured with qRT-PCR in the same samples as shown in FIG. 14A (values in both FIGS. 14A and 14B, with one set as “1” and the others adjusted accordingly, shown as means±SE from three biological replicates and asterisks denoting the significance level at P<0.01 (Student's t test), and FIG. 14C showing box and whisker plots indicative of nodule numbers in the same samples as described in FIG. 14A, with all data points represented by dots, displaying 95 to 5% interval from three biological replicates (12 plants per replicate);

FIG. 14D shows a schematic representing the structure of the artificial miRNA genes producing artificial miRNAs pursuant to the methods of the present disclosure, where such produced miRNAs are identical to two wild-type rhizobial tRF sequences;

FIG. 15, subpart A is a schematic of GFP-tRF target sites constructs, with W1-W3 representing three wild-type transgenic roots and M1-M3 representing three mutation type transgenic roots prepared according to the present disclosure, with the nucleotides within each box (□) representing cleavage sites and underlines and asterisks denoting mutations; subpart B showing graphical data resulting from the quantitation of GFP signals shown in subpart D of FIG. 15, where the GFP signals were measured using the ImageJ software and error bars indicating s.d. of GFP signals from 12 images; subpart C shows graphical data related to the reduction of expression levels of GFP fusion genes relative to that of GmEFL1b in wild-type fusion gene transgenic roots 24 hrs post inoculation (hpi) with USDA110, where GFP transcript abundance was measured by qRT-PCR and the value of one sample was set at 1 and those of the others adjusted accordingly (values shown as means±s.e. from three biological replicates, and the value of one sample set as 1 and those of the other adjusted accordingly, Bj+ indicating inoculated roots 24 hpi, Bj− indicated uninoculated roots and asterisks above columns indicative of the significance level at P<0.01 (t-test)); and subpart D shows photographs of GFP activity in transgenic roots of “GFP-tRF target site” fusion genes (W1 to W3) and “GFP-mutated tRF target site” (M1 to M3) 24 hours after inoculation with USDA110, with Bj− and Bj+ indicating uninoculated and inoculated roots, respectively;

FIG. 16 shows a gel illustrating the association of the three tRFs with soybean GmAGO1b in nodules 28 days after inoculation detected from the three experimental replicates, with “+” and “−” indicative of the GmAGO1b-Myc fusion protein-associated fraction immunoprecipitated by the Myc antibody and the nodule lysate without Myc antibody incubation, respectively;

FIGS. 17 and 18 show graphical data related to the nucleotide diversity (71) along each of the five rhizobial targets in soybean populations, with the x-axes representing the nucleotide positions of each of the five soybean genes in the soybean reference genome (cv., Williams 82, version 2), they-axes representing the levels of diversity of individual nucleotide positions among the 699 soybean accessions, and the arrows pointing to the tRF target sites within the five genes;

FIGS. 19A-19C illustrate the sequence conservation and divergence of three rhizobial tRNAs among four rhizobium groups, with FIG. 19A showing Val-1-tRNA (CAC), FIG. 19B showing Gly-1-tRNA (UCC), and FIG. 19C showing Gln-1-tRNA (CUG); and

FIGS. 20A and 20B show graphical depictions of distribution and relative abundance data of 21-nt tRFs along rhizobial tRNAs in common bean nodules, with each x-axis representing nucleotide locations of a tRNA of R. etli strain CE3 pMP604 and each y-axis indicating the frequencies of individual nucleotides along the tRNA that are covered by the 21-nt tRFs derived from the tRNA in the common bean nodules induced by CE3 pMP604.

While the present disclosure is susceptible to various modifications and alternative forms, exemplary embodiments thereof are shown by way of example in the drawings and are herein described in detail.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, systems and methods hereof may comprise many different configurations, forms, materials, and accessories.

In the following description, numerous specific details are set forth in order to provide a thorough understanding of the present disclosure. Particular examples may be implemented without some or all of these specific details and it is to be understood that this disclosure is not limited to particular biological systems, which can, of course, vary.

Unless defined otherwise, all technical and scientific terms used herein have the same meaning as commonly understood by one of skill in the relevant arts. Although any methods and materials similar to or equivalent to those described herein can be used in the practice or testing of the subject of the present application, the preferred methods and materials are described herein. To facilitate understanding of the invention, a number of terms are defined below. All publications, patents and patent applications cited herein, whether supra or infra, are hereby incorporated by reference in their entirety to the extent that the reference is not inconsistent with the teachings provided herein. Additionally, as used in this specification and the appended claims, the singular forms “a”, “an” and “the” include plural referents unless the content clearly dictates otherwise. Thus, for example, reference to “a tRNA” includes a combination of two or more tRNAs; reference to “bacteria” includes mixtures of bacteria, and the like.

Furthermore, unless specifically stated otherwise, the term “about” can allow for a degree of variability in a value or range, for example, within plus or minus 10%, within 5%, or within 1% of a stated value or limit of a range for percentages and plus or minus 1.0 unit for unit values, for example, about 1.0 refers to a range of values from 0.9 to 1.1. The term “substantially” can allow for a degree of variability in a value or range, for example, within 90%, within 95%, or within 99% of a stated value or of a stated limit of a range.

The term “plant” includes whole plants, plant organs (e.g., leaves, stems, flowers, roots, reproductive organs, embryos and parts thereof, etc.), seedlings, seeds and plant cells and progeny thereof. While the class of plants of predominant focus in the present disclosure relates to legumes, it will be understood that the inventive techniques and concepts hereof is not limited to any particular class of higher plants and the methods hereof are generally as broad as the class of higher plants amenable to transformation techniques and/or that exhibit nodulation or a similar symbiotic, cross-kingdom mechanism.

A “leguminous plant” as referred to herein is any member of the Fabaceae (or Leguminosae) family that can form nodules when infected with a rhizobial microorganism.

As used herein, the term “transgenic plants” or “transgenic plant roots” or “transgenic plant cells” refers to plants, Agrobacterium-induced plant roots, and plant cells that have DNA sequences not normally transcribed into RNA or translated into a protein (“expressed”), including, but not limited to genes that are perhaps not normally present, or any other genes or DNA sequences that one desires to introduce into the non-transformed plant, but which one desires to either genetically engineer or to have altered expression. It is contemplated that in some instances the genome of transgenic plants of the present invention will have been augmented through the stable introduction of the transgene; however, in other instances, the introduced gene will replace an endogenous sequence. A transgenic plant/root/cell includes a plant/root/cell regenerated from an originally-transformed plant or cell of the present disclosure and progeny transgenic plants from later generations or crosses of a transformed plant described herein.

A “rhizobial microorganism” as referred to herein may include any microorganism that is capable of fixing nitrogen after becoming established in a root nodule of a leguminous plant.

As used herein, “inoculating” means any method or process where a leguminous plant (including a leguminous plant seed or root) is brought into contact with a rhizobial microorganism. In some embodiments, inoculating may comprise the rhizobial microorganism being applied to the soil in which the plant is growing. In other instances, inoculating may comprise the rhizobial microorganism being applied to a portion of the plant root.

Further, as used herein, the terms “overexpression” (when used in connection with a gene), and “upregulation” have the meaning ascribed thereto by one of ordinary skill in the relevant arts, which includes (without limitation) the overexpression or misexpression of a wild-type gene product that may cause mutant phenotypes and/or lead to abundant target protein or small RNA fragment expression. “Down-regulation” or “down-regulated” may be used interchangeably and refer to a decrease in the level of a marker, such as a gene, nucleic acid, metabolite, transcript, protein, or polypeptide, as compared to an established level (e.g., that of a corresponding wild-type gene).

The terms “knockout,” “elimination,” and “deletion” in the present disclosure are used interchangeably and mean any addition or loss of a target gene sequence of a cell genome so that the protein expression mediated by the target gene is completely removed.

As used herein, “CRISPR-Cas9” means the system composed of sgRNA (guide RNA) complementarily binding to the target genome and Cas9 protein that can cut the genome gene by binding to the sgRNA and the target genome simultaneously. As a result, when the sgRNA vector and Cas9 vector are expressed temporarily in cells together concurrently, sgRNA and Cas9 protein are produced to change targeted gene sequence, leading to modification of the targeted gene at the Cas9 binding site and possible disruption of the function of the gene.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, that are synthetic, naturally occurring, and non-naturally occurring, have similar binding properties as the reference nucleic acid, and metabolized in a manner similar to the reference nucleotides.

The terms “polypeptide,” “peptide,” and “protein” are used interchangeably herein to refer to a polymer of amino acid residues, a polypeptide, or a fragment of a polypeptide, peptide, or fusion polypeptide. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the corresponding naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e. a carbon that is bound to a hydrogen, a carboxyl group, an amino group, and an R group (e.g., homoserine, norleucine, methionine sulfoxide, methionine methyl sulfonium). Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “regulatory element” means and includes, in its broadest context, a polynucleotide molecule having gene regulatory activity, i.e. one that has the ability to affect the transcription or translation of an operably linked transcribable polynucleotide molecule. Indeed, regulatory elements comprise a series of nucleotides that determine if, when, and at what level a particular gene is expressed. Regulatory elements such as promoters, leaders, introns and transcription termination regions are polynucleotide molecules having gene regulatory activity that play an integral part in the overall expression of genes in living cells. Promoters may be derived from a classical eukaryotic genomic gene, including (without limitation) the TATA box often used to achieve accurate transcription initiation, with or without a CCAAT box sequence and additional regulatory or control elements (i.e. upstream activating sequences, enhancers, and silencers) or may be the transcriptional regulatory sequences of a classical prokaryotic gene. The term “promote” may also be used herein to describe a synthetic or fusion molecule, or derivative that confers, activates, or enhances expression of a nucleic acid molecule in a cell, tissue, or organ. Promoters may contain additional copies of one or more specific regulatory elements to further enhance expression and/or to alter the spatial expression and/or temporal expression of a nucleic acid molecule, or to confer expression of a nucleic acid molecule to specific cells or tissues such as meristems, leaves, roots, embryo, flowers, seeds or fruits (i.e. a tissue-specific promoter). In the context of the present invention, a promoter preferably is a plant-expressible promoter sequence, meaning that the promoter sequence (including any additional regulatory elements added thereto or contained therein) is at least capable of inducing, conferring, activating, or enhancing expression in a plant cell, tissue or organ. Promoters that also function or solely function in non-plant cells such as bacteria, yeast cells, insect cells, and animal cells, however, are not excluded from the invention hereof.

As used herein, the term “operably linked” refers to a juxtaposition wherein the components so described are in a relationship permitting them to function in their intended manner. For example, a first polynucleotide molecule, such as a promoter, is “operably linked” with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter modulates transcription of the gene of interest in a cell.

The term “isolated” means that the material is removed from its original environment, e.g., the natural environment if it is naturally occurring. For example, a naturally-occurring polypeptide present within a living organism is not isolated, but the same polypeptide separated from some or all of the coexisting materials in the natural system is isolated. The term “purified” does not require absolute purity; instead, it is intended as a relative definition. In other words, “purified” indicates that the molecule is present in the substantial absence of other molecules of the same type. The term “purified” as used herein preferably means at least 75% by weight, more preferably at least 85% by weight, more preferably still at least 95% by weight, and most preferably at least 98% by weight, of molecules of the same type are present.

The terms “abundance” and “amount” are used interchangeably herein. “Amplification” refers to any means by which a polynucleotide sequence is copied and thus expanded into a larger number of polynucleotide molecules, e.g., by reverse transcription, polymerase chain reaction, and ligase chain reaction.

As used herein, an “analog” of a chemical compound is a compound that, by way of example, resembles another in structure but is not necessarily an isomer (e.g., 5-fluorouracil is an analog of thymine).

The term “polynucleotide” as used herein refers to a polymeric form of nucleotides of any length, either ribonucleotides or deoxyribonucleotides. This term refers only to the primary structure of the molecule and thus includes double- and single-stranded DNA and RNA. It also includes known types of modifications, for example, labels which are known in the art, methylation, “caps”, substitution of one or more of the naturally occurring nucleotides with an analog, internucleotide modifications, such as those with uncharged linkages and with charged linkages, those containing pendant moieties, such as proteins, those with intercalators, those containing chelators, those containing alkylators, those with modified linkages, as well as unmodified forms of the polynucleotide.

As used herein, the term “expression cassette” refers to a molecule comprising at least one coding sequence operably linked to a control sequence which includes all nucleotide sequences required for the transcription of cloned copies of the coding sequence and the translation of the mRNAs in an appropriate host cell. Expression cassettes can include, but are not limited to, cloning vectors, specifically designed plasmids, viruses or virus particles. The cassettes may further include an origin of replication for autonomous replication in host cells, selectable markers, various restriction sites, a potential for high copy number and strong promoters.

A “vector” is a composition of matter which comprises an isolated nucleic acid, which can be used to transfer gene sequences between cells. Thus, the term includes cloning and expression vehicles, as well as viral vectors. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base pairing rules. For example, for the sequence “A G T,” is complementary to the sequence “T C A.” Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least about 50%, 65%, 75%, 85%, 95%, 99% or more) identity over a specified region, when compared and aligned for maximum correspondence over a comparison window, or designated region as measured using a sequence comparison algorithms, or by manual alignment and visual inspection. This definition also refers to the complement of a test sequence, which has substantial sequence or subsequence complementarity when the test sequence has substantial identity to a reference sequence. This definition also refers to the complement of a test sequence, which has substantial sequence or subsequence complementarity when the test sequence has substantial identity to a reference sequence.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Substantial identity of nucleic acid sequences can be determined by comparing the sequence identity of two sequences, for example by physical/chemical methods (i.e., hybridization) or by sequence alignment via computer algorithm.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

A “compound,” as used herein, refers to a protein, polypeptide, an isolated nucleic acid, or other agent used in the method of the present disclosure.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “fragment” is a portion of an amino acid sequence, comprising at least one amino acid, or a portion of a nucleic acid sequence comprising at least one nucleotide.

As used herein, a “functional” biological molecule is a biological molecule in a form in which it exhibits a property or activity by which it is characterized. A functional enzyme, for example, is one which exhibits the characteristic catalytic activity by which the enzyme is characterized.

The phrase “level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured or discussed in terms of the actual amount of an mRNA or protein present. Such assays are coupled with processes or systems to store and process information and to help quantify levels, signals, etc. and to digitize the information for use in comparing levels.

The novel transgenic plants/roots/cells of the present disclosure are broadly directed towards transgenic roots, namely of legumes, and systems that exhibit increased nodule formation as compared to wild-type. Additionally, artificial small RNAs, identical to rhizobial-derived transfer RNA (tRNA)-derived fragments (tRFs), are provided that, when applied to and/or overexpressed in a plant, upregulate root nodule formation through repression of the plant's innate mechanisms. Methods for controlling the number of nodules on plant roots are also provided, which, in at least one embodiment, leverage the inventive transgenic roots described herein to advantageously affect crop yields.

To increase a plant's yield potential, it is important to increase the efficiencies of nodulation and nitrogen fixation. Many leguminous species can utilize atmospheric dinitrogen (N₂) gas to meet their needs for nitrogen through a symbiotic relationship with nitrogen-fixing rhizobial bacteria. Specifically, N₂ from the atmosphere is converted into ammonia (NH₃), which is then assimilated into amino acids, nucleotides, and other cellular constituents such as vitamins, flavones, and hormones. Their ability to fix gaseous nitrogen makes legumes an ideal agricultural organism as their requirement for nitrogen fertilizer is reduced.

The establishment of rhizobia-legume symbiosis is dependent on recognition of signal molecules between the partners. Upon perception of plant flavonoids, rhizobia synthesize and secrete lipochitin oligosaccharides, so called Nod factors (NF), which are perceived by root Nod factor receptors (NFR) to initiate rhizobial infection and formation of nodules where symbiotic nitrogen fixation (SNF) takes place. More specifically, the NFs initiate root hair curling, which begins with the very tip of the root hair curling around the bacteria, followed by the development of infection treads that provide a pathway for the bacteria to penetrate into the cortical cells of the roots to form nodules. Nodules are relatively distinct organs among plant species, essentially representing a controlled microbial invasion of the root. Similar to the human gut, the plant provides an environment in which specific microbes can thrive. The genetic control of nodulation development is complex, with the data presented herein showing a dependency on small RNAs (sRNAs) trafficked from shoot to root.

Because SNF is resource intensive, legumes have evolved a number of mechanisms, such as autoregulation of nodulation (AON) and nitrogen regulation of nodulation, to control the number of nodules formed. While fungal small RNA-mediated interaction with a plant has been previously reported, beyond this, previously little was known regarding if and/or how any bacterial sRNAs mediate biological processes in eukaryotes. Indeed, conventional thought has been that solely the host plant was in the driver's seat. The present disclosure leverages novel mechanisms identified by the present investigators through which this process is controlled by both symbiotic partners; bacteria indeed playing a role in nodule number modulation through use of bacteria-derived transfer RNA (tRNA)-derived fragments (tRFs).

tRNAs are one of the most conserved and abundant RNA species in cells; they function to decode messenger RNA (mRNA) into proteins in ribosomes. tRNAs have distinctive cloverlike structure, containing three hairpin loops, with a specific amino acid attached to the end that is used to translate codons in the mRNA to an amino acid transferred to the end of a growing protein. During the tRNA biogenesis process, or under specific conditions, cleavage yields tRFs. Indeed, tRFs are found in both prokaryotes and eukaryotes and were historically thought to be random degradation products, as they often accumulate in stressed or virally infected cells. This class of short, noncoding RNA fragments are less than about 30 nucleotide (nt) bases long (more particularly about 17 to about 22-nt), have few known functions, and a significant number of which are derived from precise processing at the 5′ or 3′ end of mature or precursor tRNAs.

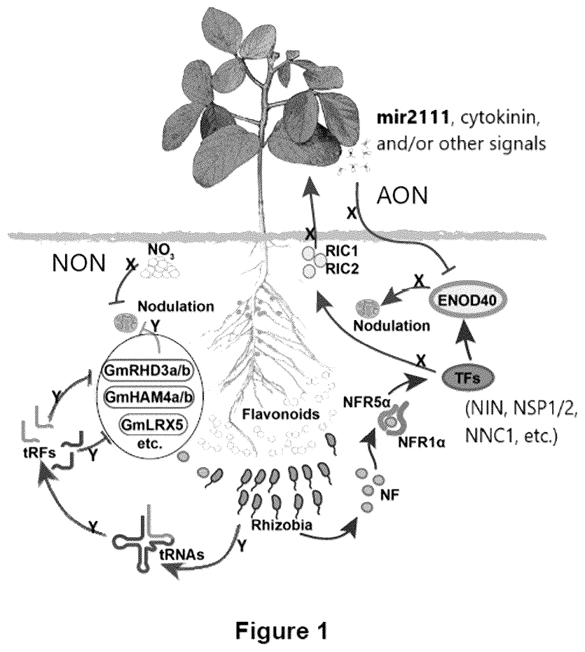

Of particular interest is that some tRFs, akin to microRNAs (miRNAs), are bound by Argonaute (AGO) proteins, indicating that they use a miRNA-like mechanism to regulate gene expression. Different from the previously thought negative regulation of nodulation by the host, the research presented in the present disclosure establishes that tRFs are positive regulators of nodulation that repress retrotransposons through an RNA interference (RNAi)-mediated silencing pathway. As shown in FIG. 1, the symbiotic balance between SNF and the growth of the host plant via tightly controlling nodule numbers is implemented through the interconnection of both the negative and positive regulatory pathways underlying nodulation.

It is contemplated that upon identifying tRFs knocked down target genes and their functions in relation to the nodule formation in plant root, regulation of these target genes' activity by CRISPR-CAS gene editing or otherwise may generate stably transgenic plants having increased nitrogen fixation efficiency. Exemplified embodiments herein are for soybean roots, but other plants suitable for such gene regulation/CRISPR-CAS editing is contemplated provided the target gene is identified.

A recent study reported that endogenous tRFs in the stem cells of mouse were able to block reverse transcription of LTR-retrotransposons further demonstrating the role of tRFs as regulatory small RNAs through RNA interference; however, it was unclear whether tRFs can target genes prior to the presently described study. Instead, conventional studies tend to focus on miRNAs and siRNAs by filtering out tRNAs and rRNAs, whereas the present study exemplifies the importance of tRFs in cross-kingdom interaction.

Perhaps more specifically, and as described in detail below, from sRNA sequencing data, multiple tRFs were identified from the rhizobium Bradyrhizobium japonicum. These tRFs were then enriched in planta and used to identify 57 target genes in the soybean (Glycine max) genome. Three of these genes—GmRHD3, GmHAM4, and GmLRX5, which are orthologs of Arabidopsis thaliana genes ROOT HAIR DEFECTIVE 3 (RHD3), HAIRY MERISTEM 4 (HAM4), and LEUCINE-RICH REPEAT EXTENSIN-LIKE 5 (LRX5), respectively—are directly involved in nodule development, including that of root hairs and were studied further as representative samples. It was then determined that the tRFs produced by the bacteria are loaded into the soybean AGO1 protein, subsequently interacting with target genes in a sequence homology-dependent manner.

As shown herein, the down-regulation of the identified target host genes by rhizobial tRFs was detected from initial rhizobial infection to nodule organogenesis. Despite their relatively low abundance, some of the rhizobial tRFs were detectable in the free-living Rhizobium culture; thus, it is most likely that the tRFs are rapidly accumulated in the rhizobial cells upon Rhizobia-host interaction and migrate into host cells where they regulate host gene expression. Besides the three rhizobial tRFs investigated in detail, additional tRFs were predicted to target candidate auxin receptors and efflux carriers, RING/U-box proteins and protein kinases, which also have ties to nodulation (Table 2). Thus, the functional roles of tRFs in regulating host nodulation are much more significant and extensive than originally thought. The identification of this novel bacterial mechanism through which bacteria regulate nodule numbers in a host plant is a significant advance in the field of symbiosis biology and bacteria-eukaryote interactions.

These new discoveries allow for the transgenic modification of one or both symbiotic partners to leverage the newly discovered cross-kingdom signaling mechanisms and confer a transgenic genotype and/or phenotype to promote and increase nodule formation (as compared to wild-type). While the present disclosure focuses on leguminous plants-Rhizobium symbiotic partners as an experimental system, it will be appreciated that the inventive concepts hereof are not so limited and any species or strain of bacteria may be employed where such bacteria is involved in a cross-kingdom symbiotic relationship with a host organism as, based on the data provided herein, it is apparent cross-kingdom communication and/or modulation via tRFs is evolutionarily conserved.

For example, and without limitation, the compounds, principles, and methods of the present disclosure may be applicable to other host-bacterial symbiotic associations such as mammalian gut microbiota and its host (whether human, animal, or otherwise). The mammalian gut represents a complex ecosystem consisting of an extraordinary number of resident commensal bacteria existing in homeostasis with the host's immune system. Akin to the symbiotic relationship between legumes and nitrogen-fixing bacteria, the host not only tolerates such bacteria, but has evolved to require such colonization for various aspects of digestion and immune development and function. While it is conventionally known that microbiota (e.g., and without limitation, Bacteroides thetaiotaomicron) provides critical signals that promote maturation of immune cells and tissues leading to protection from infections by pathogens and/or contribute to non-infectious immune disorders, heretofore the exact mechanisms of such signaling have remained unknown. Based on the data provided herein, it is contemplated that the principles of tRF signaling to host genes as disclosed herein likely apply and, thus, may be leveraged similarly to described in connection with legumes and nitrogen-fixing bacteria herein.

In at least one embodiment of the present disclosure, transgenic plants, plant roots, and/or plant cells are provided that are engineered to repress or downregulate expression of one or more nodulation-associated host genes. This may be achieved through (1) genetically engineering the host plant to directly downregulate or wholly repress one or more target genes, and/or (2) repression of such genes by engineering the host plant cells/roots to produce artificial small RNAs identical rhizobial-derived tRFs (i.e. expressing one or more polynucleotide sequences encoding RNA precursor(s) for artificial small RNA fragments that are identical to tRFs derived in wild-type Rhizobium). Indeed, the transgenic plants/plant roots/plant cells of the present disclosure may comprise one or multiple mutations directed to encoding rhizobial-derived tRFs and/or to directly repress/downregulate target genes of the plant. As such transgenic plants, plant cells or roots have a reduction in expression of the identified target genes (i.e. through rhizobial tRF mediated miRNA-like post-transcriptional regulation and/or via direct repression or downregulation of the target gene(s) themselves), any resulting transgenic root related thereto will exhibit an increased formation of nodules as compared to wild-type.

In at least one exemplary embodiment, a leguminous plant, plant root, or plant cell is modified to express a polynucleotide construct that encodes a rhizobial-derived RNA such that artificial small RNA fragments are cleaved therefrom and produced in the plant root and/or cell. The polynucleotide construct may be configured to encode a single artificial small RNA (targeting a single host gene) or a combination of small RNAs (targeting combinations of host genes, for example, GmRHD3a/GmRHD3b or GmHAM4a/GmHAM4b). In this way, the plant itself produces artificial tRFs (which are identical to those produced by wild-type rhizobium upon inoculation) that downregulate the targeted host (its own) genes that are negative regulators of nodulation herein. In this manner, the transgenic leguminous plant, plant roots, or plant cells hereof are capable of producing increased numbers of nodules as compared to nodule numbers produced from corresponding wild type legume plants, plant roots, or plant cells. Importantly, the Examples below support that the resulting small RNA fragments are identical to the tRFs produced by wild-type rhizobium and such “artificial tRFs” operate in the host root/cells in the same fashion as do rhizobial-derived tRFs.

Additionally or alternatively, the plant, plant root, or plant cell may be modified to directly downregulate or even silence one or more genes of the plant host that is a negative regulator of nodulation (i.e. the “target genes” or “host genes”). For example, this may be achieved by selective repression of the one or more host genes (using genetic modification techniques known in the art).

Furthermore, CRISPR-Cas9 or similar methodologies may be employed to knockout or delete the targeted genes of the plant/root/cell. For example, in at least one embodiment, a CRISPR-Cas9 vector may be constructed (for example and without limitation, utilizing one or more pairs of primers selected from SEQ ID NOs: 4-9 (see Table 1)) and expressed in the host plant, plant root, or plant cell such that one or more of the targeted genes of the plant/root/cell are silenced.

The target gene(s) may comprise any of the genes listed in Tables 2 and 4 (including RHD3, HAM4, and/or LRX5). Alternatively, the target gene(s) may comprise an ortholog of the foregoing including, without limitation, the following soybean (Glycine max) genes used as representative target genes for the studies disclosed herein: GmRHD3a (which encodes SEQ ID NO: 80), GmRHD3b (which encodes SEQ ID NO: 81), GmHAM4a (which encodes SEQ ID NO: 82), GmHAM4b (which encodes SEQ ID NO: 83), and GmLRX5 (which encodes SEQ ID NO: 84). As shown herein, these genes are targeted by certain tRFs and, while conventionally their function was unknown, the present disclosure establishes that they are, in fact, negative regulators of legume nodulation. In at least one exemplary embodiment, the targeted gene(s) comprises one or more of the following genes or gene combinations: GmRHD3a (which encodes SEQ ID NO: 80), GmRHD3b (which encodes SEQ ID NO: 81), GmRHD3a/GmRHD3b, GmHAM4a (which encodes SEQ ID NO: 82), GmHAM4b (which encodes SEQ ID NO: 83), GmHAM4a/GmHAM4b, GmLRX5 (which encodes SEQ ID NO: 84).

In at least one embodiment, a transgenic plant/plant root/cell may comprise a first mutation at both GmRHD3a and GmRHD3b (i.e. downregulating both genes) and a second mutation at GmLRX5. As noted above, this may be in addition to, or in lieu of, a further mutation that causes the plant/root/cell to express one or more polynucleotide sequences encoding the RNA precursor(s) for rhizobial-derived tRFs. Indeed, it will be understood by one of skill in the art that any combination of the host gene negative regulators of nodulation may be targeted for repression/downregulation, with or without any combination of expression of rhizobial-derived tRFs.

Methods of transforming cells with polynucleotide sequences, vectors, or expression cassettes that encode such compounds are well known to those skilled in the art, as are methods for selectively amplifying or repressing a host gene. Plants, plant roots, and plant cells may be transformed by, for example, electroporation, Agrobacterium transformation, engineered plant virus replicons, electrophoresis, microinjection, micro-projectile bombardment, micro-LASER beam-induced perforation of cell wall, or simply by incubation with or without polyethylene glycol (PEG).

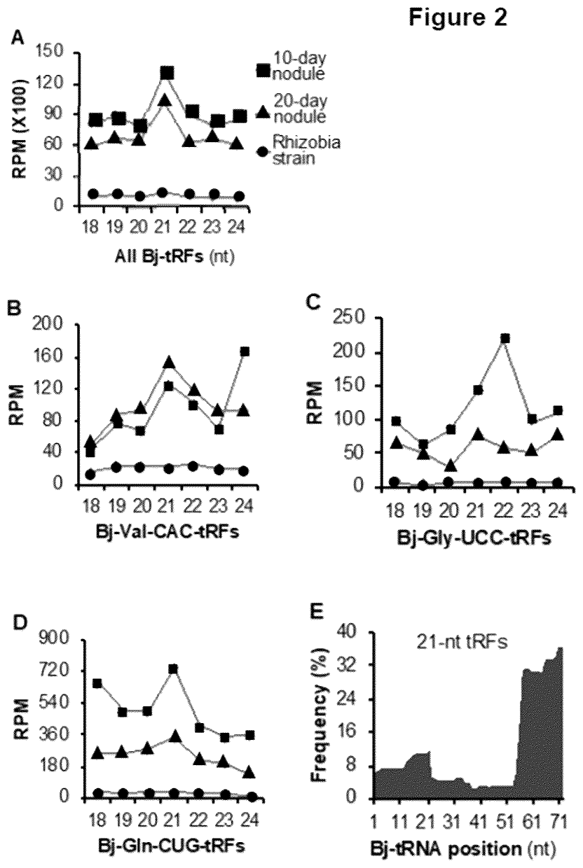

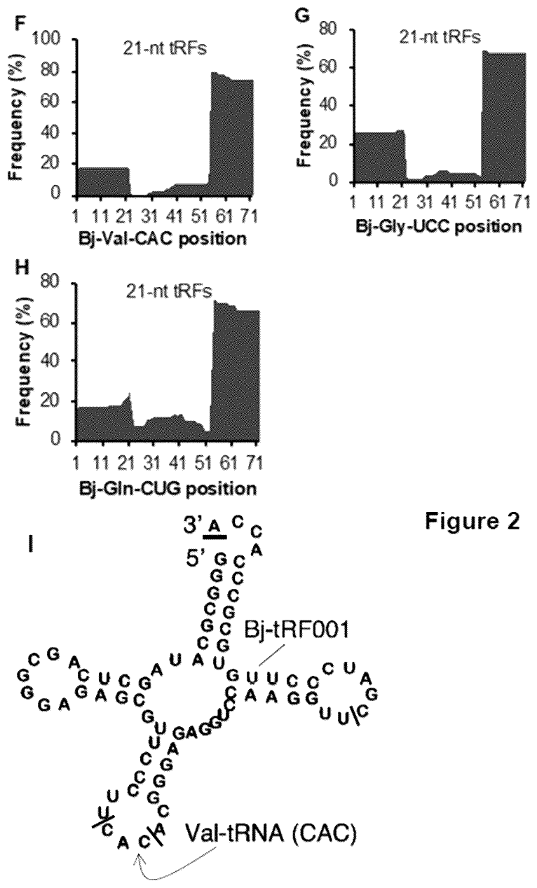

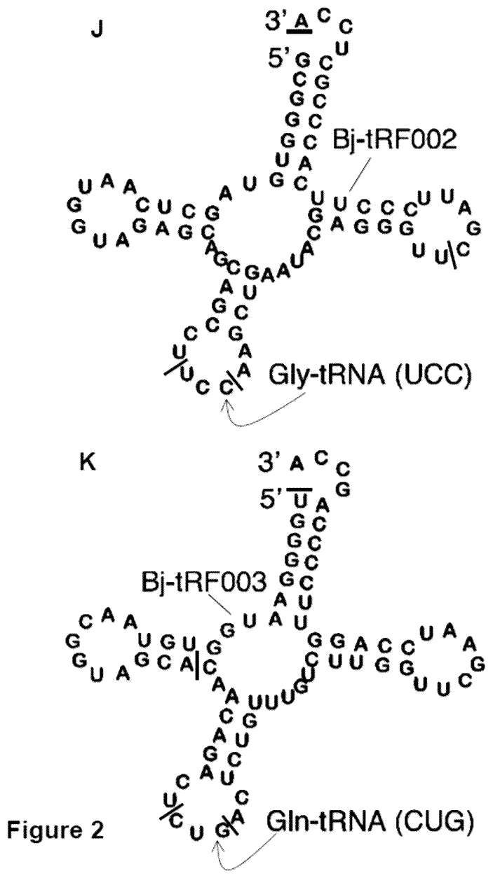

The tRFs of the present disclosure may be any tRFs that target and silence or downregulate targeted host genes through an RNAi-mediated silencing pathway in a host root nodule that inhibits nodule formation (i.e. a host negative regulator of nodulation). As such, amplification of the tRF abundance in the root nodule inhibits the plant's nodulation repression mechanism and, thus, drives increased nodule formation. In at least one embodiment, the tRFs hereof comprise any tRF listed in Tables 2 and 4 (below) or others that are otherwise capable of downregulating expression of plant host genes that are negative regulators of nodulation. Accordingly, the polynucleotide constructs described herein may comprise any of the sequences related to such tRFs. In exemplary embodiments, however, the tRFs comprise one or more of the predominant tRF products derived from three tRNAs: Val-1-tRNA(CAC), Gly-1-tRNA(UCC), and Gln-1-tRNA(CUG) (termed Bj-tRF001 (SEQ ID NO: 1), Bj-tRF002 (SEQ ID NO: 2), and Bj-tRF003 (SEQ ID NO: 3), respectively and as identified in FIG. 2, subparts I-K). Accordingly, the polynucleotide construct may comprise SEQ ID NO: 1-3, or a substantially homologous nucleic acid sequence of SEQ ID NO: 1-3.

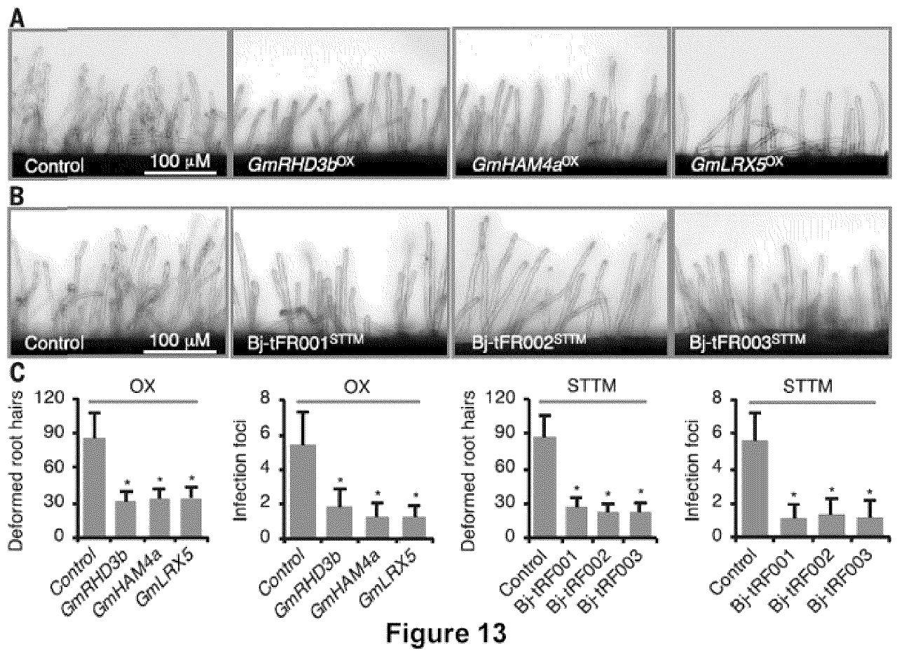

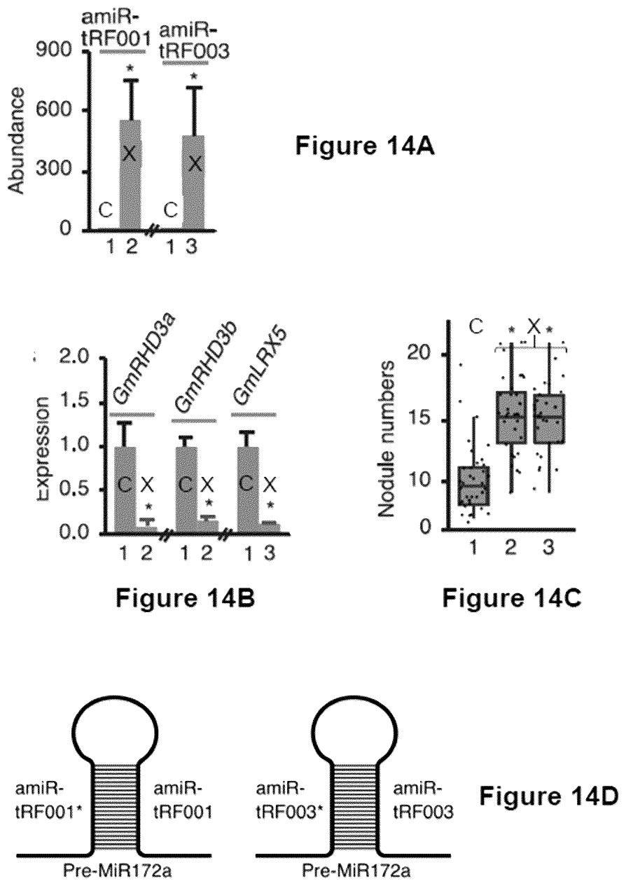

For example and without limitation, such polynucleotide constructs may encode the identified sequences, as well as the complementary sequences thereto, to produce the precursor RNA having a hairpin structure from which artificial small RNAs that are identical to rhizobial-derived tRFs are ultimately cleaved. FIG. 14D illustrates at least one exemplary embodiment of such a precursor RNA prepared pursuant to the present disclosure using a construct encoding SEQ ID NO. 1 and its complementary sequence* (left) and SEQ ID NO. 3 and its complementary sequence* (right). The RNA resulting therefrom is subsequently cleaved in planta to produce the desired artificial small RNAs which are identical to wild-type rhizobial-derived tRFs.

While specific polynucleotide sequences are provided, it will be apparent to one of ordinary skill in the art, that the disclosed artificial tRFs and/or their RNA precursors may be encoded by multiple polynucleotide sequences because of the redundancy of the genetic code. It is well within the skill of a person trained in the art to create these alternative RNA and DNA sequences that have amino acid substitutions, deletions, additions or insertions that do not materially affect biological activity of the artificial tRFs of the invention (namely, those capable of cross-kingdom communication and that initiate nodulation in plant roots). Fragments of the artificial tRFs that retain the ability to induce nodulation are also included in this definition. The polynucleotides of the present disclosure may include vectors and expression cassettes. The vectors and expression cassettes may contain transcriptional control sequences that are operably linked to polynucleotide sequences encoding the precursor RNAs and related artificial tRFs hereof.

Other embodiments of the present disclosure include genetically engineered bacterial microorganisms having upregulated production of one or more tRFs as compared to a corresponding wild-type bacterial microorganism. In at least one embodiment of the present disclosure, a species or strain of bacteria is provided that is engineered to exhibit upregulated expression of one or more tRFs as compared to such production in a wild-type of the corresponding species or strain. The one or more amplified tRFs may be any tRFs capable of cross-kingdom communication and/or signaling with at least one target gene of host organism.

For example, and without limitation, the rhizobial microorganism may express a polynucleotide construct that enhances the microorganism's production of one or more tRFs such as those identified in Tables 2 and 4 of the present disclosure. The polynucleotide construct may be configured to upregulate expression of a single tRF (targeting a single host gene) or a combination of tRFs (targeting combinations of host genes, for example, GmRHD3a/GmRHD3b or GmHAM4a/GmHAM4b).

In at least one exemplary embodiment, the host organism is a leguminous plant, plant root, or plant cell, such as a soybean (e.g., Glycine max) and may comprise the transgenic leguminous plants, plant roots, or plant cells previously described or a wild-type leguminous plant. In any event, the bacterial-derived tRFs are capable of repressing or downregulating expression of at least one gene target in the host organism's nodules. There, the one or more tRFs may be, for example, tRFs that target and silence or repress a target host through an RNAi-mediated silencing pathway in the root nodule designed to inhibit nodule formation.

Where the host organism is a leguminous plant, the bacterial microorganism may be of any strain or species of nitrogen-fixing bacteria such as, for example and without limitation, a rhizobial microorganism such as a species or strain of B. japonicum USDA 110. Other non-limiting examples of such rhizobial microorganisms include other B. japonicum strains (e.g., such as USDA 123 and 138), Rhizobium etli, Sinorhizobium meliloti, Rhizobium leguminosarum, Parasponia rhizobium, Mesorhizobium loti, and other compatible rhizobium species.

Compounds comprising one or more species or strains of the bacterial microorganisms hereof are also disclosed. In at least one embodiment, the compound is formulated in an inoculant composition comprising the engineered bacterial microorganism as heretofore described. The compound may comprise a single species/strain of the engineered bacterial microorganisms of the present disclosure or a combination of different species and/or strains of bacterial microorganisms in the compound. Engineered bacteria may additionally be combined with wild-type bacteria strains or species, or all bacteria within the compound may be genetically modified (with the same or different mutations) to amplify expression of one or more tRFs as described herein.

The inoculant composition may comprise a carrier or additive. The carrier or additive used will depend on the nature of the inoculant composition. For example, the inoculant composition may be in the form of a liquid composition, a solid composition (such as a powder, pellet or granular composition), a seed coating, or in any other form suitable for the desired application. In at least one embodiment, the inoculant composition comprises a liquid composition and includes a solvent suitable for dissolving or suspending the compound.

The inoculant compositions of the present disclosure may be adapted for application to a leguminous plant, plant root or cell in any suitable way. For example, the inoculant composition could be adapted to be applied as a seed coating, applied as a solid or liquid composition to the foliage or roots of a plant, or applied as a solid or liquid composition to the soil before, during or after sowing of a leguminous plant. A range of useful carriers or additives would be readily apparent to those of skill in the art and may include, for example: one or more gums (including xanthan gum), clay or peat based carriers, one or more nutrients including carbon or nitrogen sources, one or more antifungal or antibacterial agents, one or more seed coating agents, one or more wetting agents and the like.

Methods of increasing nodulation activity in leguminous plant roots may be practiced in transgenic plants/plant roots/plant cells that are engineered to repress or downregulate the identified target genes and in non-transgenic plants to which the engineered bacterial microorganisms and/or inoculants are applied. Further, methods are also provided that utilize both approaches concurrently—i.e. application of the engineered bacterial microorganisms having amplified tRF production and/or inoculants to the transgenic plants/plant roots/plant cells of the present disclosure.

In at least one embodiment, such a method comprises repressing or downregulating expression of one or more target genes of a host plant root or cell, and inoculating, or having inoculated, the modified plant host root or cell with at least one rhizobium to initiate nodulation. The inoculating step may be performed pursuant to known methodologies and using conventional microorganism species and strains. In one embodiment, the roots may be wounded to enable the bacterial cells to penetrate the roots more quickly and easily; however, wounding of the roots is not required.

The target gene(s) may comprise any of the target genes described herein and, in at least one exemplary embodiment, comprise RHD3, HAM4, LRX5, an ortholog of RHD3, an ortholog of HAM4, and/or an ortholog of LRX5. Perhaps more specifically, and in yet another exemplary embodiment where the leguminous plant root is Glycine max, each host gene may comprise GmRHD3a (which encodes SEQ ID NO: 80), GmRHD3b (which encodes SEQ ID NO: 81), GmHAM4a (which encodes SEQ ID NO: 82), GmHAM4b (which encodes SEQ ID NO: 83), or GmLRX5 (which encodes SEQ ID NO: 84).

The species or strains of the at least one rhizobium can likewise comprise any microorganism that initiates nodulation in a host plant root or cell including, for example, Bradyrhizobium japonicum, Rhizobium etli, Sinorhizobium meliloti, Rhizobium leguminosarum, Parasponia rhizobium, Mesorhizobium loti and the like.

The step of downregulating or repressing expression of the one or more target genes of the host plant root or cell may comprise introducing and expressing a first polynucleotide construct that encodes a rhizobial-derived RNA that is the precursor for producing one or more artificial tRFs in the plant root as previously described. In at least one exemplary embodiment, the RNA may have a hairpin structure as shown in FIG. 14D. The first polynucleotide construct may encode one or more of SEQ ID NOs. 1-3, 34-70, or any substantially homologous nucleic acid sequence to any of the foregoing. Further, the first construct may also encode a complementary sequence to the aforementioned (similar to as seen in FIG. 14D as associated with the resulting hairpin structure).

Additionally or alternatively, downregulating or repressing expression of the one or more target genes of the host plant root or cell may comprise constructing a CRISPR-Cas9 vector as described herein and expressing the vector in the host plant root or cell, where such vector is designed to silence one or more of the target host genes identified herein. In at least one exemplary embodiment, the CRISPR vector may be designed to silence one or more of the following: GmRHD3a, GmRHD3b, GmRHD3a/GmRHD3b, GmHAM4a, GmHAM4b, GmHAM4a/GmHAM4b, and GmLRX5.

Still further, rather than silencing a host gene via the CRISPR-Cas9 methodology, the step of downregulating or repressing expression of the one or more target genes of the host plant root or cell may comprise repressing expression of one or more of the targeted host genes. Such repressed expression can be achieved through any suitable methodology now or hereinafter known in the art and, in at least one embodiment may comprise expressing a polynucleotide construct in the host root or cell that encodes a transcription factor for downregulating or repressing expression of the one or more host genes. Furthermore, such a method may involve application of the compounds and/or compositions having the engineered bacterial microorganisms directly to the transgenic roots or the roots of the transgenic plants, plant roots, or plant cells described herein. In one embodiment, the roots may be wounded to enable the bacterial cells to penetrate the roots more quickly and easily; however, wounding of the roots is not required.

Due to the confirmed ability of the inventive compounds, plants/roots/cells, and methods of the present disclosure to increase nodule formation in leguminous plants, application of the concepts set forth herein can significantly reduce the use of nitrogen-based fertilizers, cutting down the economic and environmental cost of agriculture, without diminishing yield. The present disclosure is further described with reference to the following non-limiting examples.

### Materials and Methods

For the examples described herein, the following materials and methods were used.

### Plant and Rhizobium Materials

Soybean (Glycine max) cultivar Williams 8 were grown in a greenhouse under 16 h light/8 h dark period for RNA isolation and hairy root transformation, and root and nodule tissue collection. Williams 82 was also used for preparation of protoplasts from soybean roots. Bradyrhizobium japonicum strain USDA110 was cultured in a modified arabinose gluconate (MAG) medium composed of 1.3 g HEPES, 1.1 g MES, 1 g yeast extract, 1 g L-arabinose, 1 g D-gluconic acid sodium salt, 0.22 g KH₂PO₄, and 0.25 g Na₂SO₄ per liter, pH 6.6, and used for soybean root inoculation.

Soybean seeds were germinated in sterilized vermiculite sand mixture (3:1) for 7 days before inoculation with the Rhizobium culture or treatment with the MAG medium without the Rhizobia (control sets). The largest nodules on inoculated roots 10 days and 20 days post inoculation (DPI or dpi) and uninoculated roots at the same stages were collected for RNA isolation. Root hair cells were isolated from the roots following a protocol set forth in Lin et al., Molecular response to the pathogen Phytophthora sojae among ten soybean near isogenic lines revealed by comparative transcriptomics, BMC Genomics, 15: 18 (2014).

### DNA and RNA Isolation, PCR, and Sequencing

Genomic DNA isolation, polymerase chain reaction (PCR), reverse transcription-PCR (RT-PCR), quantitative RT-PCR (qRT-PCR), and sequencing of DNA fragments was performed as previously described in Ping et al., Dt2 is a gain-of-function MADS-domain factor gene that specifies semideterminacy in soybean, Plant Cell, 26: 2831-2842 (2014). In qRT-PCR, the soybean gene GmELF1b (Glyma.02G276600) or GmCons4 (Glyma.12g020500) was used as an internal reference to quantify the relative expression levels of the rhizobial tRF targets identified using three biological replicates.

Stem-loop RT-PCR was performed to evaluate relative abundance of rhizobial tRFs. The specificity of stem-loop RT-PCR for individual tRFs was confirmed by sequencing of the amplified fragments. The b11631 gene in B. japonicum USDA 110 was used as an internal reference to quantify the relative abundance of tRFs determined by stem-loop RT-PCR using three biological replicates. The primers used for the varieties of PCR and sequencing of amplified fragments were developed using strategies and techniques known in the art and are listed in the Supplemental Materials of the article Ren et al., Rhizobial tRNA-derived small RNAs are signal molecules regulating plant modulation, Science 365, 919-922 (2019) (including all Supplemental Materials related thereto, the “Ren et al. Article”)), the entirety of which is incorporated herein by reference, with the specific primers used for CRISPR-Cas9 and amplification studies listed in Table 1.

TABLE 1
Primers used with CRISPR-Cas9 and amplification studies.

| Primers | Sequence (5′-3′) | Purpose |
|---|---|---|
| RHD3_pSC_F | SEQ ID NO: 4 | CRISPR-Cas9 construction |
| RHD3_pSC_B | SEQ ID NO: 5 | CRISPR-Cas9 construction |
| HAM4_pSC_F | SEQ ID NO: 6 | CRISPR-Cas9 construction |
| HAM4_pSC_B | SEQ ID NO: 7 | CRISPR-Cas9 construction |
| LRX5_pSC-F | SEQ ID NO: 8 | CRISPR-Cas9 construction |
| LRX5_pSC-B | SEQ ID NO: 9 | CRISPR-Cas9 construction |
| RHD3b_CDS_F | SEQ ID NO: 74 | overexpression construction |
| RHD3b_CDS_B | SEQ ID NO: 75 | overexpression construction |
| HAM4a_CDS_F | SEQ ID NO: 76 | overexpression construction |
| HAM4a_CDS_B | SEQ ID NO: 77 | overexpression construction |
| LRX5_CDS_F | SEQ ID NO: 78 | overexpression construction |
| LRX5_CDS_B | SEQ ID NO: 79 | overexpression construction |

sRNA-Seg and mRNA-Seg & Data Analyses

Total NRAs from plant roots, root nodules, and Rhizobia were isolated using TRIzol Reagent (Invitrogen/Life Technologies, CA). The small RNA-seq (sRNA-seq) and mRNA libraries were constructed using the TruSeq small-RNA kit and TruSeq Stranded mRNA kit (Illumina, CA), respectively, according to the manufacturer's protocol, and sequenced using HiSeq2500 (Illumina, CA) at the Purdue Genomics Core Facility (West Lafayette, IN).

Raw sRNA-seq data were processed with the FASTQ/A short-reads pre-processing tools, FASTX-Toolkit, for removal of adapter sequences and low-quality reads. The processed reads longer than 16 nt were compared with the soybean reference genome sequence (Version 2, Phytozome) and the soybean chloroplast and mitochondria sequences using Bowtie (version 1.0.0) with a parameter of “-v 0.” The reads unmapped to these soybean sequences were subsequently compared with the B. japonicum USDA110 reference genome sequence using Bowtie. The reads perfectly mapped to the USDA110 genome sequence without any mismatches were further annotated and those perfectly matching transfer RNA (tRNA) sequences of USDA 110 were considered as rhizobial tRNA derived fragments (tRFs). Relative abundance of unique rhizobial tRFs in each library were normalized to counts per million reads (CPM). Unique rhizobial tRFs (Bj-tRFs) with a relative abundance higher than 100 CPM in at least one of the nodule sRNA-seq libraries were used to identify soybean gene targets of rhizobial tRFs.

Raw mRNA-seq data were also processed with the FASTX-Toolkit for removal of adapter sequences and low-quality reads. The processed reads from each library were mapped to the soybean reference genome (Version 2, Phytozome) using TopHat2 mapping tool (version 2.1.1), with a parameter that allowed for one mismatch per read. Relative expression level of an individual soybean gene was determined based on the number of reads uniquely mapped to corresponding genes using the BEDTools software (version 2.27.1). Differentially expressed genes (DEGs) between compared samples were defined using “edgeR” (version 3.22.5), with an adjusted false discovery rate (FDR)-controlled q-value<0.05. A subset of DEGs was further validated by qRT-PCR.

### Sequence Alignment and Phylogenetic Analysis

Sequence alignment and construction of a Neighbor-Joining phylogenetic tree were performed using a procedure described in Niu et al., Expression of artificial microRNAs in transgenic Arabidopsis thaliana confers virus resistance, Nat. Biotechnol., 24: 1420-1428 (2006). The tree was visualized using Evolview, an online visualization tool for phylogenetic trees (version 2).

### tRF Target Gene Prediction and Target Site Validation

psRNA Target, a plant small RNA target analysis server, was used to predict the target genes of rhizobial tRFs. A predicted tRF target was considered as a strong candidate when the expectation value determined by the server was no more than 3 and when the candidate gene exhibited differential expression between the root and nodule samples. 5′ RNA ligase mediated rapid amplification of cDNA ends (RLM-RACE) was performed to identify cleavage sites of mRNAs from the rhizobial tRF targets using the Gene Racer RLM-RACE Kit (Invitrogen, MA) according to the manufacturer's protocol. The cDNA samples were amplified by nested PCR. The PCR products were cloned into the pGEM-T easy vector (Promega, WI) and 40 clones for each target site were sequenced at Purdue Genomics Core Facility (West Lafayette, IN) to determine the cleavage site(s) in the transcripts of the tRF target(s).

### Plasmid Construction and Soybean Hairy Root Transformation

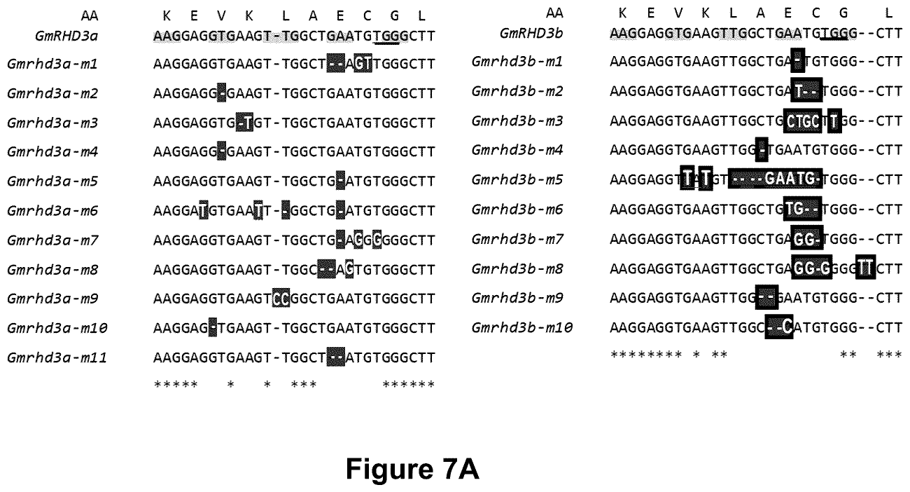

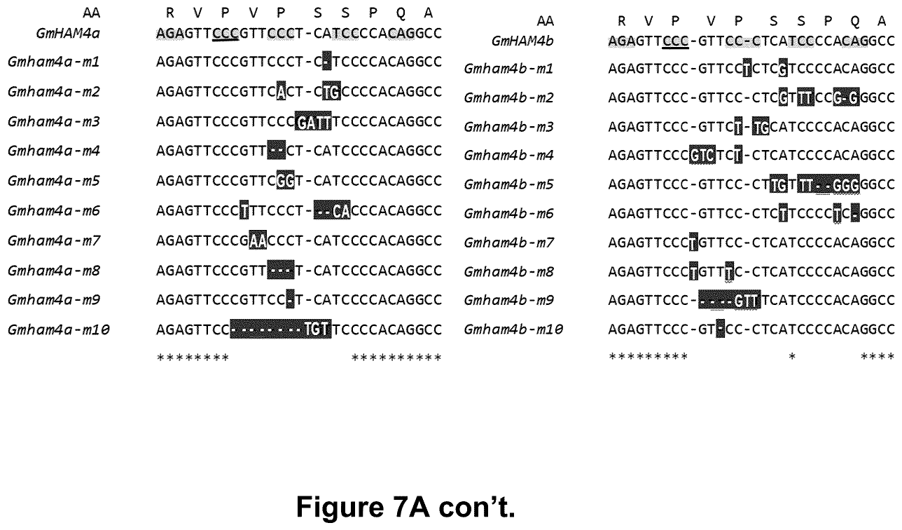

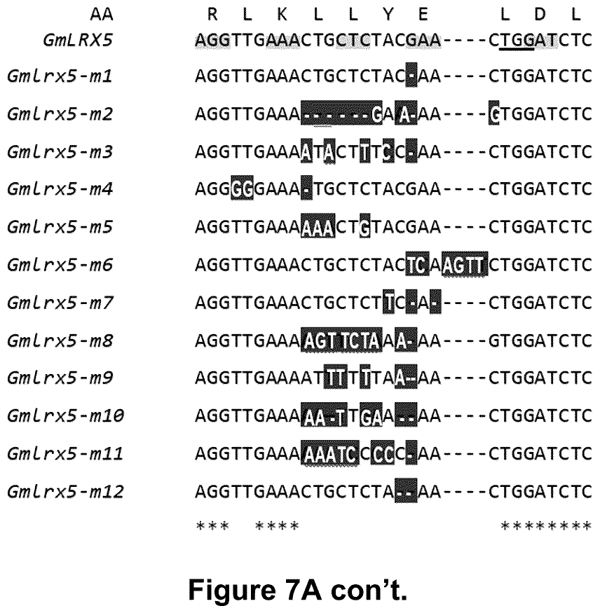

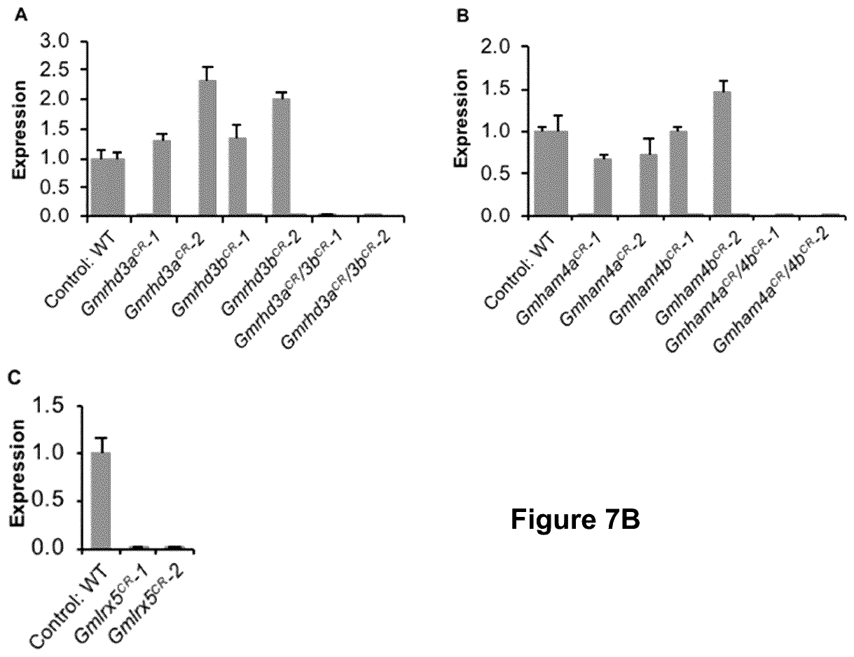

The pSC1 vector was used to develop gRNA-Cas9 expression vectors following a protocol described in Rueden et al., ImageJ2: ImageJ for the next generation of scientific image data,” BMC Bioinformatics, 18: 529 (2017). sgRNAs for editing the five soybean genes GmRHD3a 3b, GmHAM4a 4b, and GmLRX5 were designed using CRISPR-P, a web-based guided RNA design tool. The primer pairs used for this study were annealed (95° C. for 5 min and then cool down to room temperature) to form dimers, and then integrated into the Lgu I-digested pSC1 vector, separately. The sgRNAs targets exons of the genes as shown in FIG. 7.

The pBIN438 vector was used to develop the overexpression constructs under the control of a cauliflower mosaic virus 35S promoter for soybean genes GmRHD3b, GmHAM4a, and GmLRX5. The GmRHD3b and GmLRX5 coding sequences (CDS) were amplified by the primers listed on Table 1, respectively, and then integrated into pBIN438 using the Sal I/Xba I enzymes, and the GmHAM4a CDS was inserted into pBIN438 using the Xho I Kpn I enzymes.

The pEGAD vector was used to develop three STTM constructs, each silencing a rhizobial tRF in soybean hairy roots. Individual STTM modules were designed based on the three 21-nt rhizobial tRFs. The primer pairs containing the STTM nodules were annealed to dimers, and then integrated separately into pEGAD using the Age I Bam HI enzymes. All primers used for plasmid construction are listed in the Ren et al. Article.

Soybean hairy root transformation was performed as previously described with minor modification. Hypocotyls of one-week-old soybean seedlings were injected with Agrobacterium rhizogenes K599 and kept at high humidity (>90%) with plastic lids until hairy roots at the injection sites were developed to 5-10 cm long. The original seedling main roots were then cut and the hairy roots were immersed in water for about five days before the plants with hairy roots were transferred into the pots with sterilized vermiculite and sand mixture (3:1). Two days after the plant transfer into pots, the plant roots were inoculated with the B. japonicum strain USDA110 (20 mL suspension (OD=0.05) per pot). Transgenic roots were identified by PCR of unique sequences from respective plasmid constructs and further validated by sequencing of PCR fragments. CRISPR-Cas9 induced knockout hairy roots were identified by sequencing of amplified gene fragments harboring the target sites of the designed guided RNAs that were cloned into the pGEM-T easy vector (Promega, WI). Only hairy roots with both alleles mutated at the target sites were considered as knockout hairy roots for phenotyping studies.

### Phenotyping of Root Nodule Numbers

Nodules on transgenic hairy roots and the controls were counted 28 dpi. Approximately one-cm root segments in root hair zone of the six dpi-hairy roots were cut and washed with distilled deionized water and then fixed with ethanol: acetic acid (3:1) for two hours. The fixed roots were examined for root hair morphology including deformation and welling tips or wavy growth and curling (formation of infection foci), and root hair numbers and length, and were photographed with a Nikon Eclipse Ti2 microscope (Nikon, NY).

### Example 1

### Verification of Cross-Kingdom Communication

To address if tRFs are involved in cross-kingdom communications, soybean-Rhizobium symbiotic partners were used as an experimental system. In particular, the soybean (Glycine max) and the rhizobium (Bradyrhizobium japonicum) were studied as symbiotic partners. Given the lack of AGO-modified RNAi machinery in bacteria, the intent was to identify tRFs derived from rhizobial tRNAs that regulate soybean genes. Small RNAs and mRNAs from B. japonicum strain USDA110, 10-day and 20-day old soybean (cv., Williams 82) nodules induced by USDA110, and uninoculated Williams 82 roots were isolated and sequenced using the protocols described above, and putative tRFs produced by Rhizobia either in the Rhizobium culture or in the Rhizobia-induced nodules were identified.

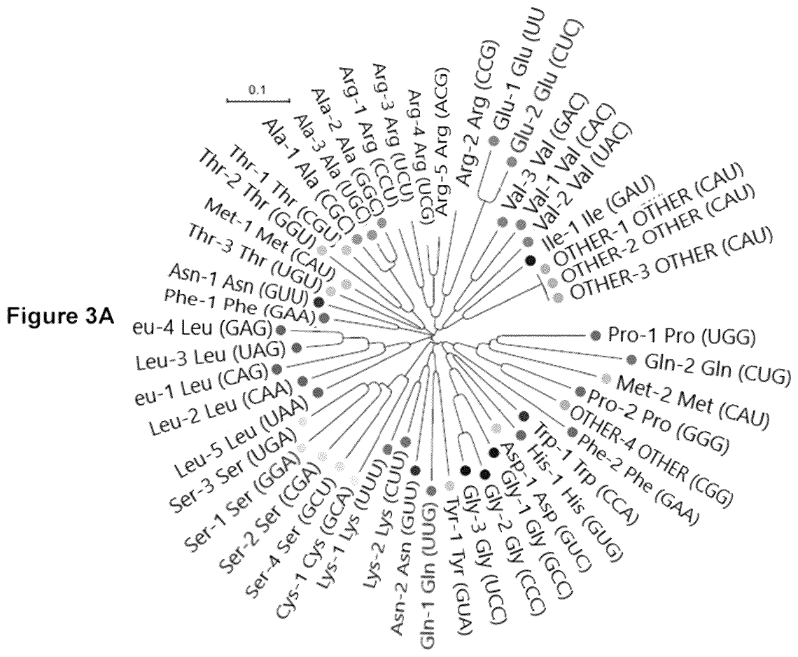

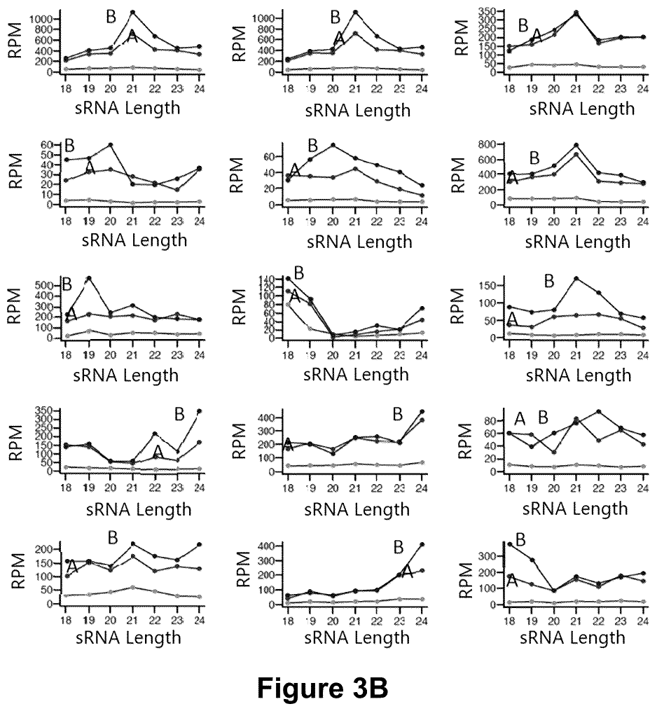

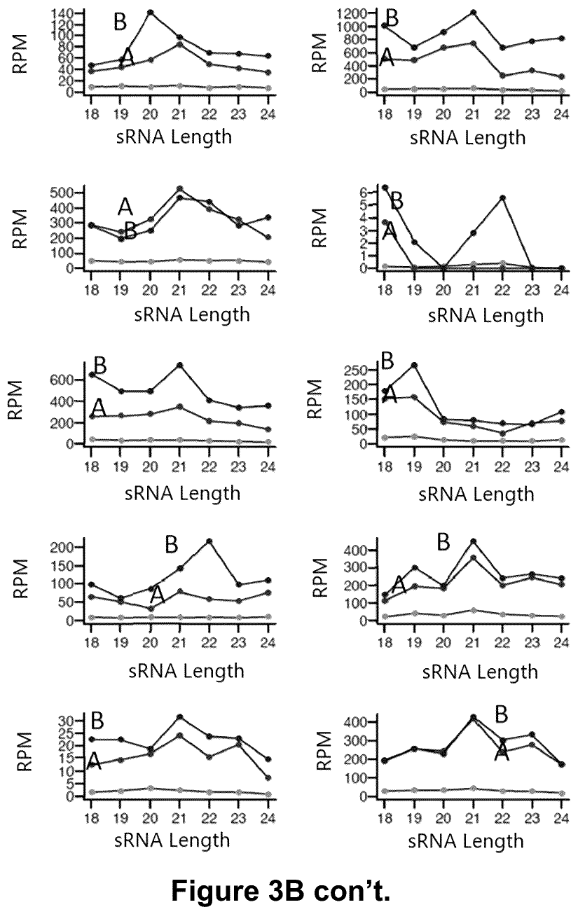

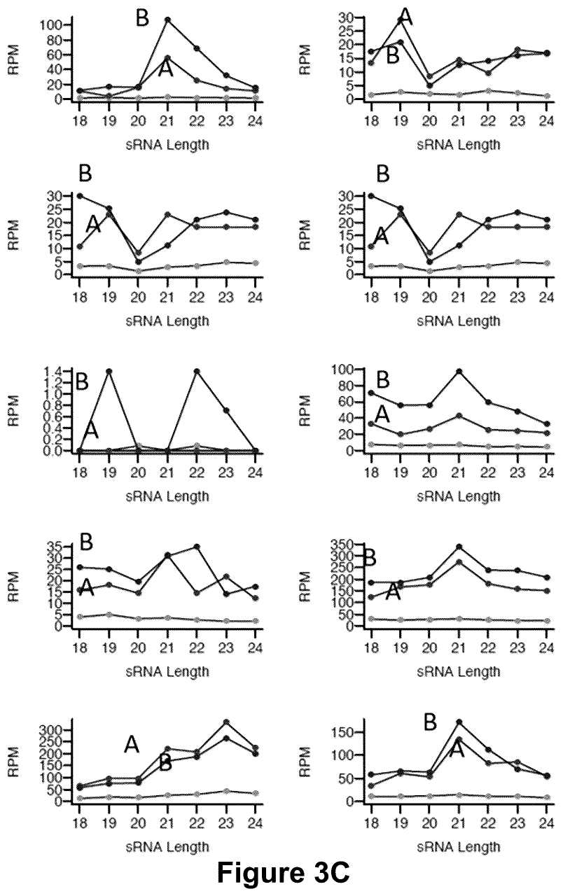

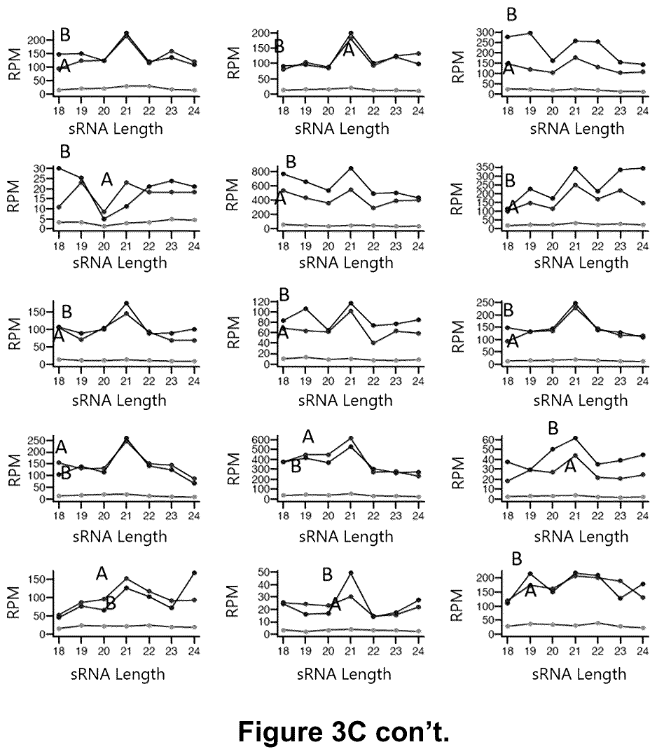

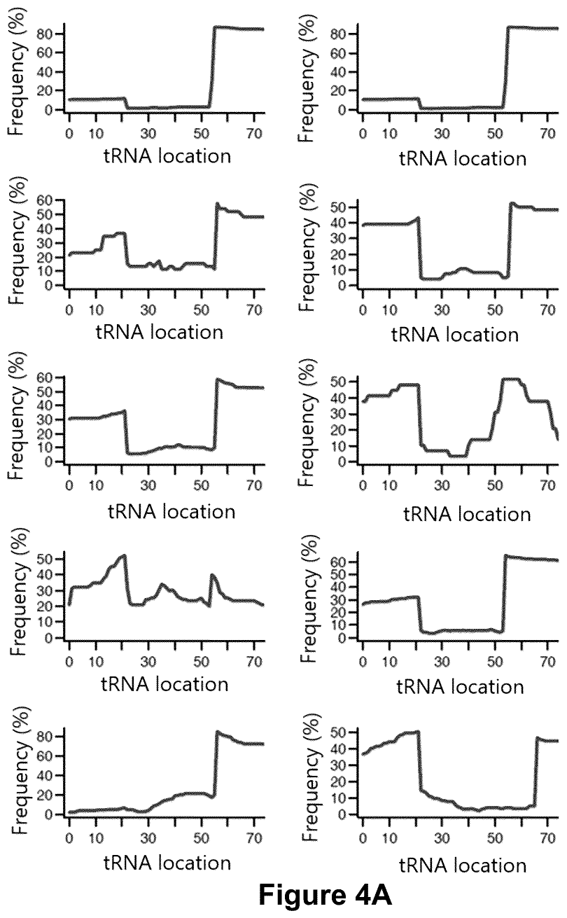

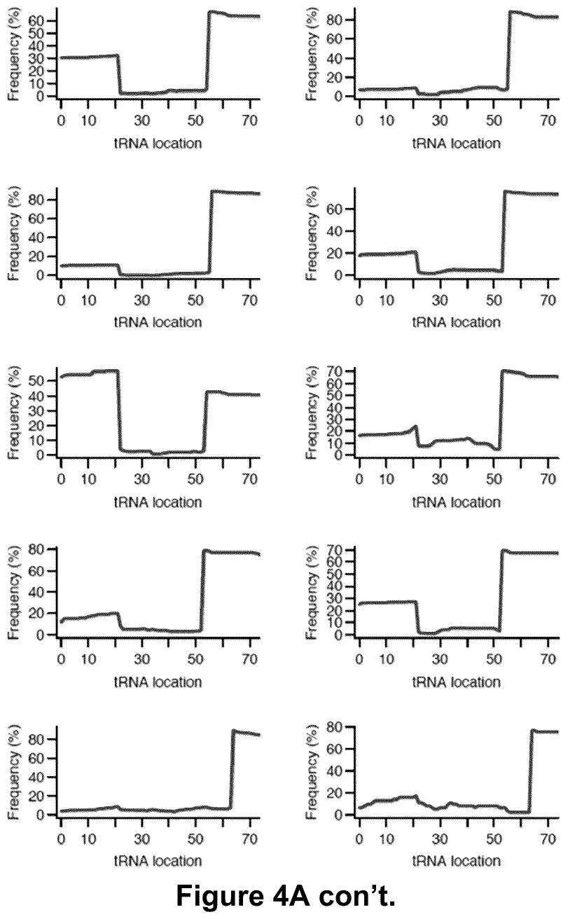

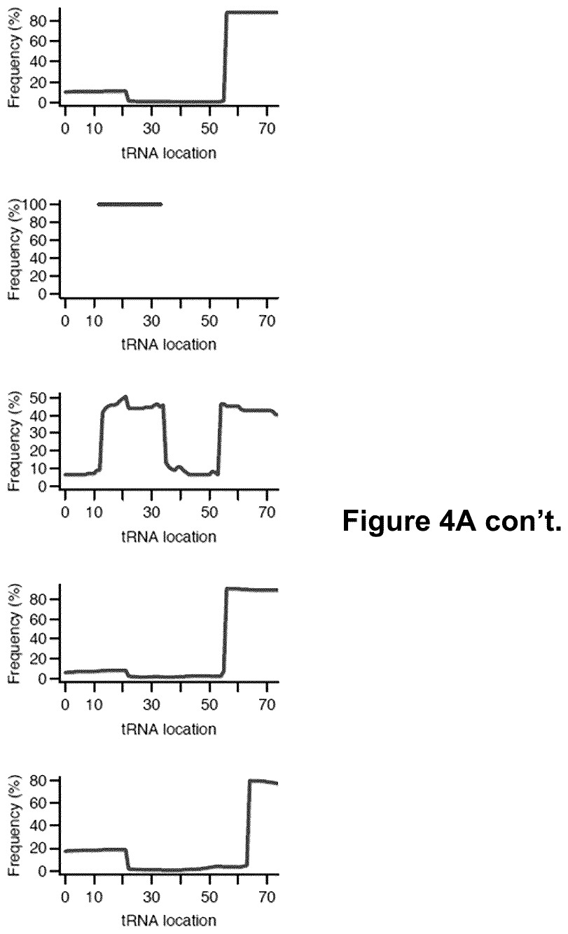

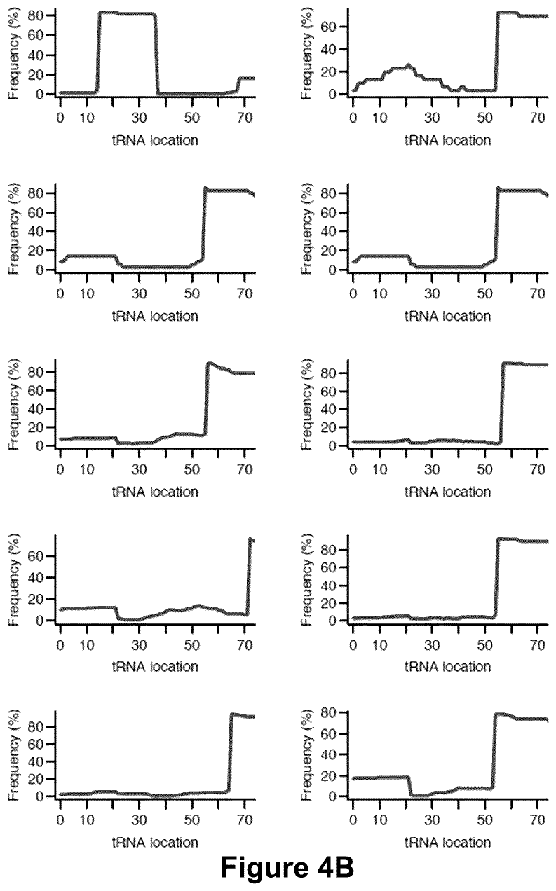

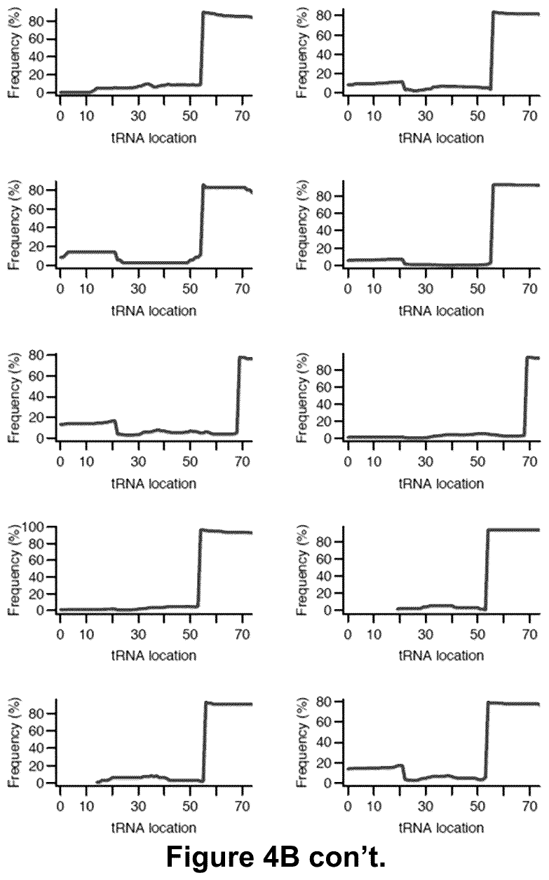

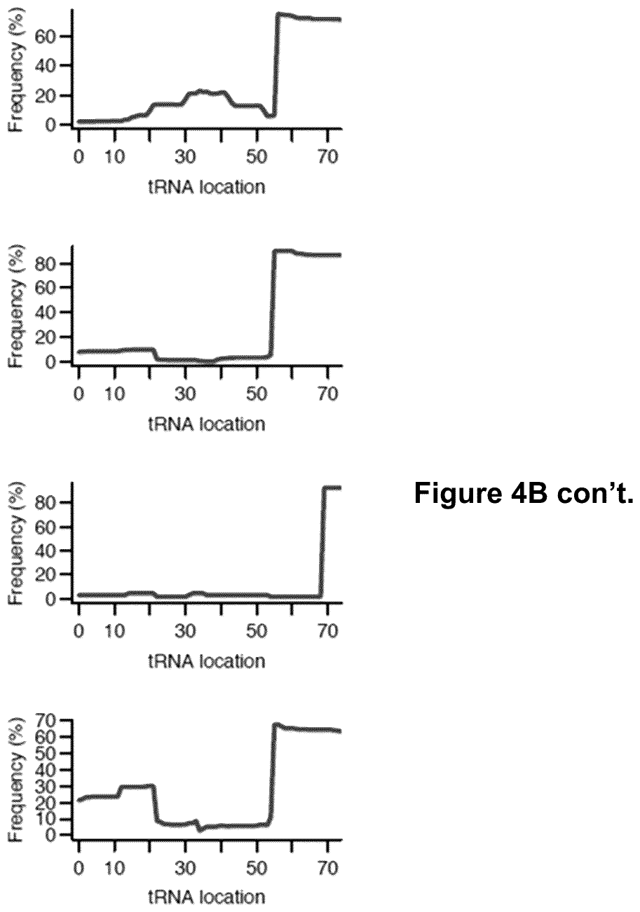

All 50 rhizobial tRNAs produced tRFs in both Rhizobium (strain USDA110) culture and the 10-day-old and 20-day-old soybean (cultivar Williams 82) nodules, with the majority ranging from 18 to 24 nt in size, and abundance varied drastically (see FIGS. 2A and 3A and Table 2). Overall, the tRFs in the nodules were significantly more abundant than those in the Rhizobium culture, with 21-nt tRFs—primarily derived from the 3′ ends of the tRNAs—most abundant (see FIGS. 2A and 3A-3C).

A total of 57 soybean genes in the soybean genome were predicted to be targets of 25 distinct 21- or 22-nt rhizobial tRFs, with a relative abundance of >100 copies per million rhizobial small RNA reads (Table 2, with host target sequences being the complementary sequence to that listed as sRNA-seq).

TABLE 2
Rhizobial tRFs derived from B. japonicum and their potential targets in soybean.

| sRNA | target gene | sRNA-seq | tRNA-name | |
|---|---|---|---|---|
| Bj-tRF001 | Glyma.07G010200 | SEQ ID NO: 1 | BJ-tRNA-Val-1 | RHD3 |
| Bj-tRF001 | Glyma.08G193200 | SEQ ID NO: 1 | BJ-tRNA-Val-1 | RHD3 |
| Bj-tRF001 | Glyma.01G189600 | SEQ ID NO: 1 | BJ-tRNA-Val-1 | SKP1/ASK1-interacting |
| | | | | protein 2 |
| Bj-tRF001 | Glyma.11G052600 | SEQ ID NO: 1 | BJ-tRNA-Val-1 | SKP1/ASK1-interacting |
| | | | | protein 2 |
| Bj-tRF002 | Glyma.06G108100 | SEQ ID NO: 2 | BJ-tRNA-Gly-3 | Galactosyltransferase family |
| | | | | protein |
| Bj-tRF002 | Glyma.04G254100 | SEQ ID NO: 2 | BJ-tRNA-Gly-3 | Galactosyltransferase family |
| | | | | protein |
| Bj-tRF002 | Glyma.11G065200 | SEQ ID NO: 2 | BJ-tRNA-Gly-3 | GRAS family transcription |
| | | | | factor |
| Bj-tRF002 | Glyma.01G177200 | SEQ ID NO: 2 | BJ-tRNA-Gly-3 | GRAS family transcription |
| | | | | factor |
| Bj-tRF002 | Glyma.02G058700 | SEQ ID NO: 2 | BJ-tRNA-Gly-3 | GRAS family transcription |
| | | | | factor |
| Bj-tRF002 | Glyma.16G141300 | SEQ ID NO: 2 | BJ-tRNA-Gly-3 | GRAS family transcription |
| | | | | factor |
| Bj-tRF003 | Glyma.08G039400 | SEQ ID NO: 3 | BJ-tRNA-Gln-2 | LRR receptor-like |
| | | | | serine/threonine-protein |
| | | | | kinase |
| Bj-tRF106 | Glyma.07G007700 | SEQ ID NO: 50 | BJ-tRNA-Met-2 | ATPase E1-E2 type family |
| | | | | protein |
| Bj-tRF107 | Glyma.19G242600 | SEQ ID NO: 51 | BJ-tRNA-Pro-2 | DNA-binding storekeeper |
| | | | | protein-related transcriptional |
| | | | | regulator |
| Bj-tRF107 | Glyma.04G054600 | SEQ ID NO: 51 | BJ-tRNA-Pro-2 | Mitochondrial substrate |
| | | | | carrier family protein |
| Bj-tRF107 | Glyma.18G278400 | SEQ ID NO: 51 | BJ-tRNA-Pro-2 | Pentatricopeptide repeat |
| | | | | (PPR) superfamily protein |
| Bj-tRF107 | Glyma.13G073900 | SEQ ID NO: 51 | BJ-tRNA-Pro-2 | protein kinase 2A |
| Bj-tRF109 | Glyma.10G021500 | SEQ ID NO: 52 | BJ-tRNA-Thr-2 | F-box/RNI-like superfamily |
| | | | | protein |
| Bj-tRF144 | Glyma.11G053000 | SEQ ID NO: 53 | BJ-tRNA-Pro-1 | Nucleotide-diphospho-sugar |
| | | | | transferases superfamily |
| | | | | protein |
| Bj-tRF144 | Glyma.01G189200 | SEQ ID NO: 53 | BJ-tRNA-Pro-1 | Nucleotide-diphospho-sugar |
| | | | | transferases superfamily |
| | | | | protein |
| Bj-tRF145 | Glyma.17G021200 | SEQ ID NO: 54 | BJ-tRNA-Gln-2 | 2-oxoglutarate (2OG) and |
| | | | | Fe(II)-dependent oxygenase |
| | | | | superfamily protein |
| Bj-tRF145 | Glyma.05G015400 | SEQ ID NO: 54 | BJ-tRNA-Gln-2 | ATPase E1-E2 type family |
| | | | | protein |
| Bj-tRF145 | Glyma.06G017300 | SEQ ID NO: 54 | BJ-tRNA-Gln-2 | DNA-binding bromodomain- |
| | | | | containing protein |
| Bj-tRF147 | Glyma.13G050800 | SEQ ID NO: 55 | BJ-tRNA-Ser-1 | RING/U-box superfamily |
| | | | | protein |
| Bj-tRF147 | Glyma.19G037800 | SEQ ID NO: 55 | BJ-tRNA-Ser-1 | RING/U-box superfamily |
| | | | | protein |
| Bj-tRF148 | Glyma.05G005600 | SEQ ID NO: 56 | BJ-tRNA-Ser-2 | domains rearranged |
| | | | | methyltransferase 2 |
| Bj-tRF148 | Glyma.13G050800 | SEQ ID NO: 56 | BJ-tRNA-Ser-2 | RING/U-box superfamily |
| | | | | protein |
| Bj-tRF148 | Glyma.19G037800 | SEQ ID NO: 56 | BJ-tRNA-Ser-2 | RING/U-box superfamily |
| | | | | protein |
| Bj-tRF151 | Glyma.15G007900 | SEQ ID NO: 57 | BJ-tRNA-OTHER-4 | Tetratricopeptide repeat |
| | | | | (TPR)-like superfamily |
| | | | | protein |
| Bj-tRF154 | Glyma.06G185400 | SEQ ID NO: 58 | BJ-tRNA-Arg-2 | plant U-box 38 |
| Bj-tRF157 | Glyma.11G089100 | SEQ ID NO: 59 | BJ-tRNA-His-1 | Concanavalin A-like lectin |
| | | | | protein kinase family protein |
| Bj-tRF160 | Glyma.09G191700 | SEQ ID NO: 60 | BJ-tRNA-Trp-1 | Enoyl-CoA |
| | | | | hydratase/isomerase family |
| Bj-tRF160 | Glyma.11G105900 | SEQ ID NO: 60 | BJ-tRNA-Trp-1 | glycine-rich protein 2B |
| Bj-tRF160 | Glyma.03G238400 | SEQ ID NO: 60 | BJ-tRNA-Trp-1 | Integral membrane Yip1 |
| | | | | family protein |
| Bj-tRF160 | Glyma.19G235900 | SEQ ID NO: 60 | BJ-tRNA-Trp-1 | Integral membrane Yip1 |
| | | | | family protein |
| Bj-tRF163 | Glyma.20G114800 | SEQ ID NO: 61 | BJ-tRNA-Arg-1 | Hypothetical protein |
| Bj-tRF164 | Glyma.04G092100 | SEQ ID NO: 62 | BJ-tRNA-Leu-4 | dicarboxylate carrier 2 |
| Bj-tRF171 | Glyma.19G189300 | SEQ ID NO: 63 | BJ-tRNA-Ala-3 | Exostosin family protein |
| Bj-tRF173 | Glyma.02G034300 | SEQ ID NO: 64 | BJ-tRNA-Val-3 | splicing factor PWI domain- |
| | | | | containing protein |
| Bj-tRF173 | Glyma.04G236700 | SEQ ID NO: 64 | BJ-tRNA-Val-3 | Hypothetical protein |
| Bj-tRF176 | Glyma.06G108100 | SEQ ID NO: 65 | BJ-tRNA-Gly-2 | Galactosyltransferase family |
| | | | | protein |
| Bj-tRF176 | Glyma.04G254100 | SEQ ID NO: 65 | BJ-tRNA-Gly-2 | Galactosyltransferase family |
| | | | | protein |
| Bj-tRF176 | Glyma.11G065200 | SEQ ID NO: 65 | BJ-tRNA-Gly-2 | GRAS family transcription |
| | | | | factor |
| Bj-tRF176 | Glyma.01G177200 | SEQ ID NO: 65 | BJ-tRNA-Gly-2 | GRAS family transcription |
| | | | | factor |
| Bj-tRF177 | Glyma.18G085100 | SEQ ID NO: 66 | BJ-tRNA-Phe-1 | beta-1,2-xylosyltransferase |
| Bj-tRF320 | Glyma.11G087300 | SEQ ID NO: 67 | BJ-tRNA-Leu-4 | Auxin efflux carrier family |
| | | | | protein |
| Bj-tRF320 | Glyma.04G255100 | SEQ ID NO: 67 | BJ-tRNA-Leu-4 | Coatomer, alpha subunit |
| | | | | cysteine-rich RLK |
| Bj-tRF344 | Glyma.10G253300 | SEQ ID NO: 68 | BJ-tRNA-Gly-1 | (RECEPTOR-like protein |
| | | | | kinase) 29 |
| Bj-tRF344 | Glyma.07G044400 | SEQ ID NO: 68 | BJ-tRNA-Gly-1 | Subtilase family protein |
| Bj-tRF344 | Glyma.16G012700 | SEQ ID NO: 68 | BJ-tRNA-Gly-1 | Subtilase family protein |
| Bj-tRF550 | Glyma.10G092500 | SEQ ID NO: 69 | BJ-tRNA-Asn-1 | cytochrome P450, family 93, |
| | | | | subfamily D, polypeptide 1 |
| Bj-tRF550 | Glyma.20G209700 | SEQ ID NO: 69 | BJ-tRNA-Asn-1 | myb domain protein 15 |
| Bj-tRF550 | Glyma.14G082600 | SEQ ID NO: 69 | BJ-tRNA-Asn-1 | Plant protein of unknown |
| | | | | function (DUF827) |
| Bj-tRF550 | Glyma.06G023300 | SEQ ID NO: 69 | BJ-tRNA-Asn-1 | RAC-like 3 |
| Bj-tRF597 | Glyma.19G127000 | SEQ ID No: 3 | BJ-tRNA-Gln-2 | Homeodomain-like |
| | | | | superfamily protein |
| Bj-tRF597 | Glyma.05G190300 | SEQ ID No: 3 | BJ-tRNA-Gln-2 | Plant invertase/pectin |
| | | | | methylesterase inhibitor |
| | | | | superfamily |
| Bj-tRF597 | Glyma.18G176600 | SEQ ID No: 3 | BJ-tRNA-Gln-2 | XB3 ortholog 1 in |
| | | | | Arabidopsis thaliana |
| Bj-tRF656 | Glyma.07G269000 | SEQ ID NO: 70 | BJ-tRNA-Leu-5 | C2 calcium/lipid-binding and |
| | | | | GRAM domain containing |
| | | | | protein |

A total of 68 soybean genes, which also showed at least two-fold reduction of expression in nodules (as compared with uninoculated roots) were defined as the targets of 36 unique tRFs from 25 rhizobial tRNAs (Table 2). These tRFs were neither found in small RNA libraries from non-nodule soybean tissues (NCBI accession no. GSE58779; Table 3 (representative data based on Arikit et al., An atlas of soybean small RNAs identifies phased siRNAs from hundreds of coding genes, Plant Cell 26: 4584-4601 (2014), the entirety of which is incorporated herein by reference) nor conventionally predicted to target rhizobial genes.

TABLE 3
Presence/absence of B. japonicum-derived tRFs in different tissues of soybean.

| tRF Sequences | Nod10_1 | Nod10_3 | Nod15 | Nod20 | Nod25 | Nod30 | AntW82 |
|---|---|---|---|---|---|---|---|
| SEQ ID NO: 10 | 419 | 569 | 1011 | 1925 | 1021 | 834 | 0 |
| SEQ ID NO: 11 | 265 | 372 | 340 | 731 | 284 | 265 | 0 |
| SEQ ID NO: 12 | 3530 | 6450 | 3234 | 5231 | 2102 | 1603 | 0 |
| SEQ ID NO: 13 | 60 | 95 | 63 | 145 | 69 | 126 | 0 |
| SEQ ID NO: 14 | 268 | 659 | 726 | 1557 | 662 | 478 | 0 |
| SEQ ID NO: 15 | 489 | 437 | 209 | 667 | 325 | 275 | 0 |
| SEQ ID NO: 16 | 1142 | 1382 | 577 | 1240 | 494 | 732 | 0 |
| SEQ ID NO: 17 | 974 | 1170 | 621 | 1021 | 619 | 846 | 0 |
| SEQ ID NO: 18 | 1955 | 1903 | 1868 | 2859 | 1519 | 1078 | 0 |
| SEQ ID NO: 19 | 414 | 367 | 231 | 450 | 174 | 238 | 0 |
| SEQ ID NO: 20 | 383 | 408 | 199 | 346 | 154 | 191 | 0 |
| SEQ ID NO: 21 | 139 | 253 | 165 | 357 | 124 | 158 | 0 |
| SEQ ID NO: 22 | 6079 | 7235 | 4324 | 8686 | 5301 | 7720 | 0 |
| SEQ ID NO: 23 | 1067 | 634 | 326 | 538 | 289 | 754 | 0 |
| SEQ ID NO: 24 | 150 | 227 | 175 | 315 | 113 | 120 | 0 |
| SEQ ID NO: 25 | 79 | 126 | 79 | 164 | 76 | 72 | 0 |
| SEQ ID NO: 26 | 163 | 182 | 81 | 211 | 99 | 152 | 0 |
| SEQ ID NO: 27 | 206 | 274 | 115 | 289 | 117 | 126 | 0 |
| SEQ ID NO: 28 | 2301 | 2744 | 3270 | 7007 | 3333 | 2424 | 0 |
| SEQ ID NO: 29 | 0 | 9 | 12 | 33 | 12 | 8 | 0 |
| SEQ ID NO: 30 | 58 | 100 | 48 | 93 | 49 | 61 | 0 |
| SEQ ID NO: 31 | 9 | 18 | 7 | 14 | 7 | 15 | 0 |
| SEQ ID NO: 32 | 7 | 11 | 8 | 15 | 19 | 18 | 0 |
| SEQ ID NO: 33 | 27 | 18 | 11 | 16 | 19 | 145 | 0 |

Referring back to Table 2, of the soybean genes, GmRHD3a/GmRHD3b, GmHAM4a/GmHAM4b, and GmLRX5—which are orthologs of the Arabidopsis Root Hair Defective 3 (RHD3), Hairy Meristem 4 (HAM4), and Leucine-Rich Repeat Extension-Like 5 (LRX5), respectively—were identified for further study, in part, because these Arabidopsis genes are important for root hair and plant development. Indeed, these soybean genes were predicted to be the targets of three rhizobial tRFs-dubbed Bj-tRF001, Bj-tRF002, and Bj-tRF003—which are the predominant products derived from three tRNAs: Val-1-tRNA(CAC), Gly-1-tRNA(UCC), and Gln-1-tRNA(CUG), respectively (see FIG. 2, subparts I-K).

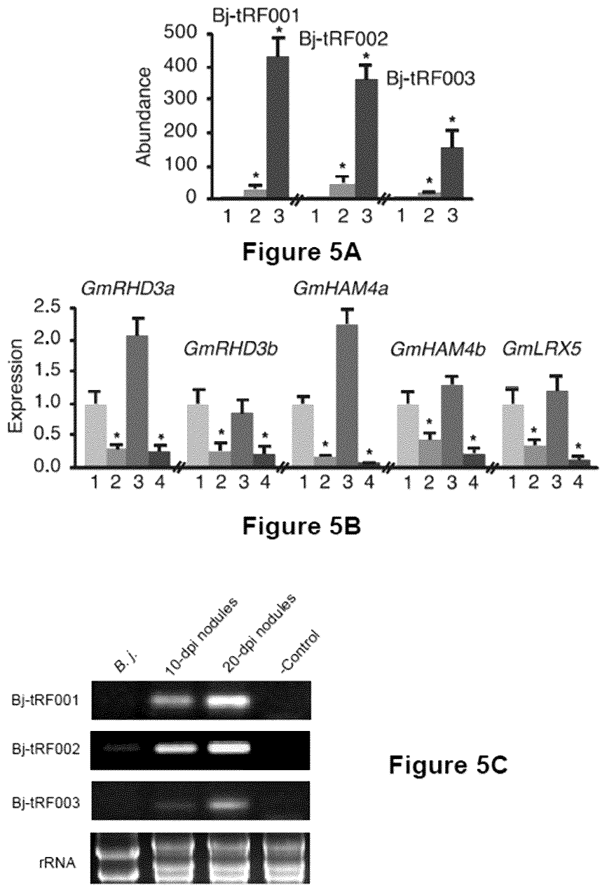

Enrichment of the three identified rhizobial tRFs in the nodules compared with the uninoculated Rhizobium culture (i.e. the transcripts from a B. japonicum gene b11631 in the free-living (wild-type) B.j. strain USDA110) was further validated by means of stem-loop RT-qPCR assay (see FIGS. 5A and 5C). Specifically, abundance of these three rhizobial tRFs in the 10-dpi and 20-dpi nodules was detected (x-axis labeled 2 and 3 in FIG. 5A, respectively). A reaction from the 20-dpi RNA sample without adding reverse transcriptase served as a negative control (x-axis labeled 1 in FIG. 5A).

The specificity of stem-loop RT-PCR products from individual tRFs was then confirmed by sequencing the amplified fragments. Equal amounts of total RNAs from different samples were loaded onto the gel to visualize different sizes and relative abundances of rhizobial rRNAs and soybean rRNAs. As expected, the reduced expression of their candidate gene targets in the nodules as compared with the uninoculated roots was revealed (see FIG. 5B).

### Example 2

Target Genes' Cleavage Site in the Transcripts and Suppression by tRFs

To confirm that the identified soybean genes were the targets of the three rhizobial tRFs, cleavage sites in the transcripts of these genes were identified by RNA ligase-mediated 5′ rapid amplification of cDNA ends (RLM-RACE). If the reduced expression of these soybean genes was caused by the identified rhizobial tRFs through miRNA-like posttranscriptional regulation, cleavage of the mRNAs from these genes at the predicted tRF target sites would occur.

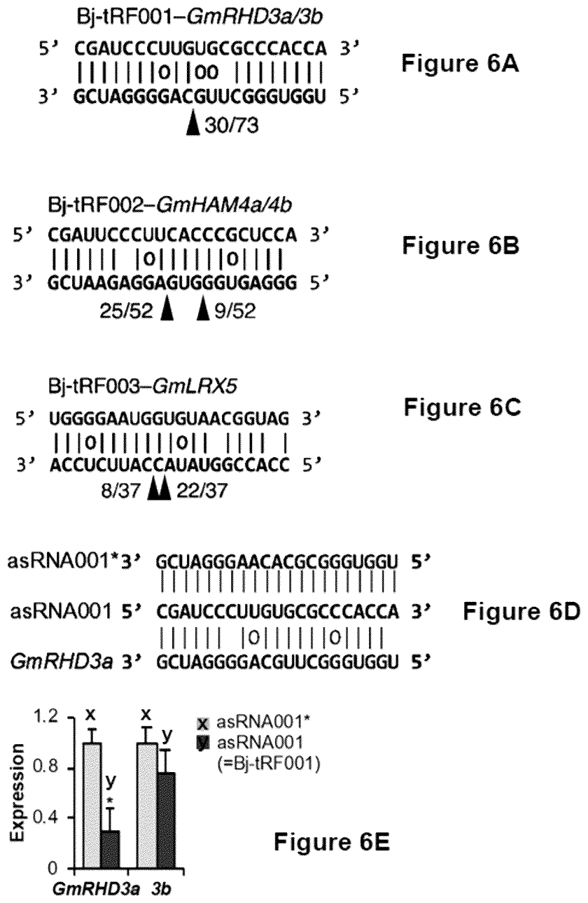

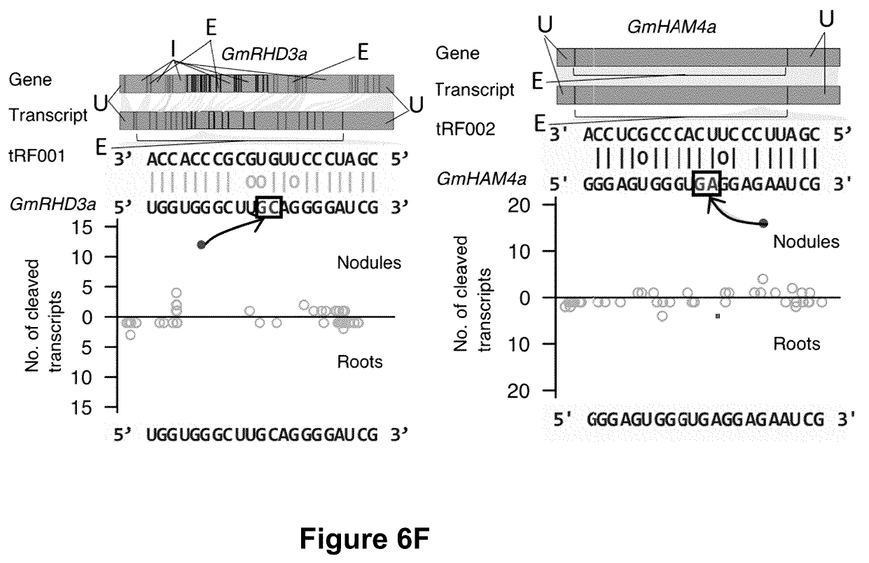

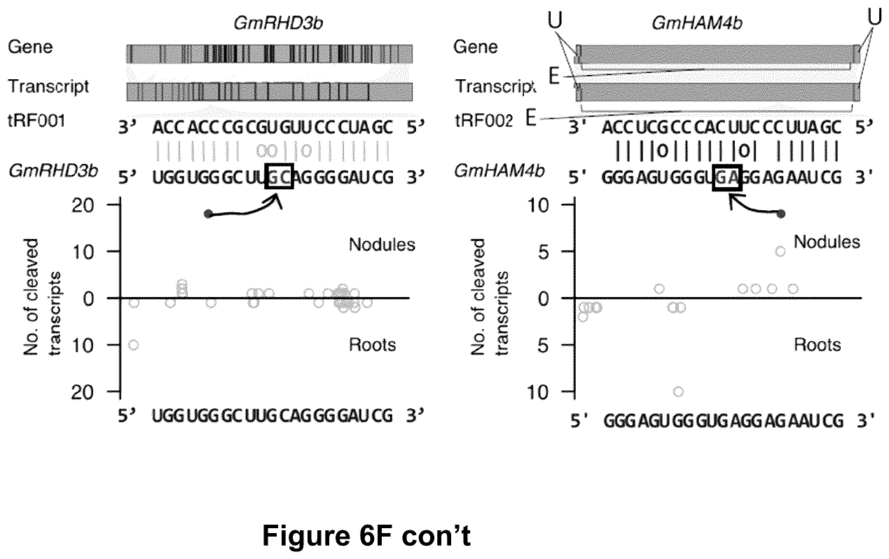

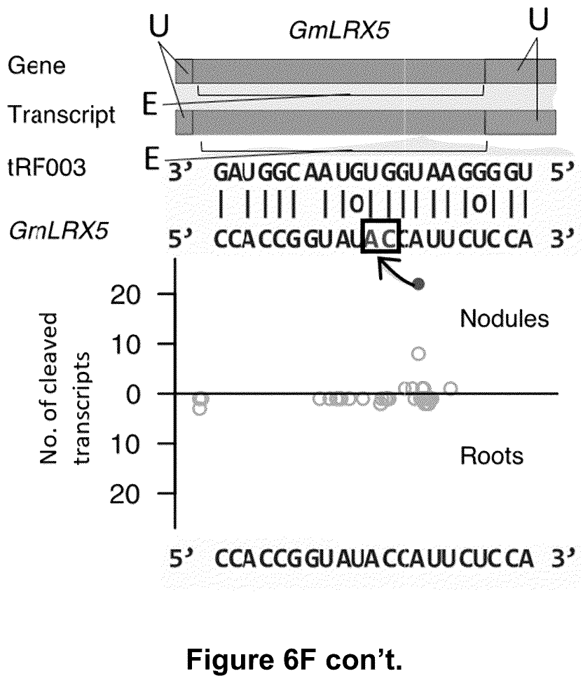

Examining this, the data supports that the mRNAs of these genes were in fact exactly cleaved at the predicted tRF target sites in the 20-day nodules, whereas none of these sites were cleaved in the uninoculated roots (see FIGS. 6A-6C and 6E). This clearly supports these tRFs repress the expression of the target genes by a miRNA-like post-transcriptional regulatory mechanism.

Importantly, none of these sites were complementary to or, prior to this study, predicted to be targeted by previously identified soybean small RNAs. Indeed, soybean miR171k was the only small RNA predicted to target GmHAM4a/GmHAM4b, but it was primarily expressed in the uninoculated roots (9.38 counts per million reads) instead of the nodules (0.27 counts per million reads) and, thus, unlikely to be responsible for the observed repression of GmHAM4a/GmHAM4b in the nodules. Accordingly, the data indicates that there is, in fact, cross-kingdom communication between the newly identified rhizobial tRFs and the soybean genes thus supporting that the rhizobial tRFs do participate in the regulation of nodulation.

To understand the significance of individual tRFs in repressing its target(s), two artificial small RNAs, asRNA001 (identical to Bj-tRF001) and asRNA001* (complementary to asRNA001) (FIG. 6D) were synthesized and transformed separately into the protoplasts prepared from soybean (cv., Williams 82) roots grown in sterile tissue culture. Significant or substantial reductions of the transcripts of both GmRHD3a and GmRHD3b were detected in the asRNA001 transformation protoplast as compared to these in the asRNA001* transformation protoplast (FIG. 6E), supporting that an individual 21-nt rhizobial tRF is sufficient for post-transcriptional repression of its target.

### Example 3

Mutations of tRFs' Target Genes Resulted in Nodule Initiation

Heretofore, the potential functions of GmRHD3a/GmRHD3b, GmHAM4a/GmHAM4b, and GmLRX5 in soybean and their homologs in other legumes were unknown. As these genes were downregulated by rhizobial tRFs only detected locally in the root nodules of soybean as previously described, it was contemplated that these genes are associated with nodulation. To determine whether the repression of GmRHD3a/GmRHD3b, GmHAM4a/GmHAM4b, and GmLRX5 expression in the nodules is, in fact, associated with nodulation, root mutants were created by means of CRISPR-Cas9 (CR) (FIGS. 7A and 7B) through hairy root transformation for each of the five genes and for both copies of each of the two duplicated gene pairs, followed by inoculation with Rhizobium strain USDA 110. The controls were transgenic roots of empty vectors used for the CR knockouts.

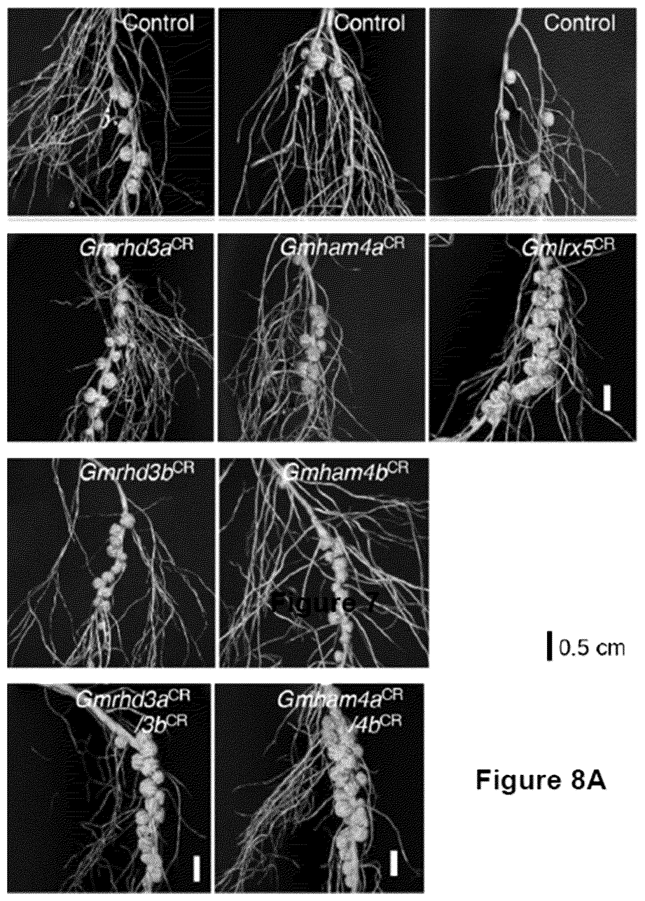

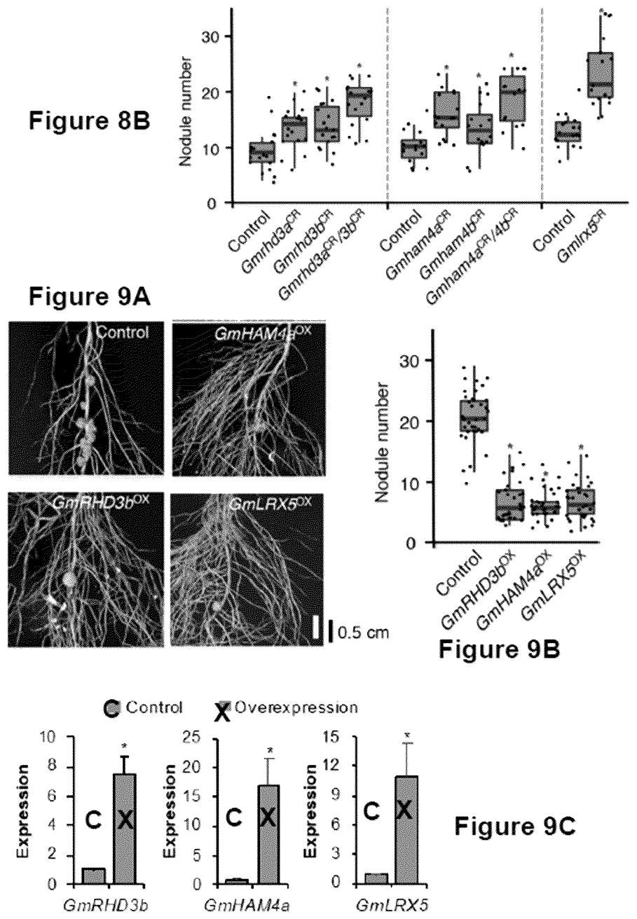

In all cases, expression of the edited genes produced more nodules than those of the empty-vector transgenic controls. Furthermore, the double mutants (Gmrhd3a/Gmrhd3b and Gmham4a/Gmham4b) produced the greatest number of nodules. FIG. 8A shows photographs of the roots of each mutant and the controls, with all mutants having knockouts of the identified tRF targets by means of CR resulting in significantly increased nodule numbers. FIG. 8B additionally shows a graphical representation of the resulting nodule numbers.

### Example 4

Overexpression of tRFs' Target Genes Reduces Nodule Numbers

Overexpression of GmRHD3b, GmHAM4a, and GmLRX5 in the nodules was also examined with respect to its effect on nodulation. Transgenic roots were prepared that overexpressed GmRHD3b, GmHAM4a, or GmLRX5, separately, by the cauliflower mosaic virus (CaMV) 35S promoter, which increased expression of such genes (i.e. the tRF targets). As seen in FIGS. 9A-9C, overexpression of these genes resulted in a dramatic reduction in nodule numbers in the gene-overexpression roots as compared to the control non-transgenic roots. This, taken with the data of the other studies presented herein, supports that these genes are negative regulators of nodulation.

### Example 5

Rhizobial tRFs Directly Act on Nodulation Through Cleavage of Transcripts of Target Genes

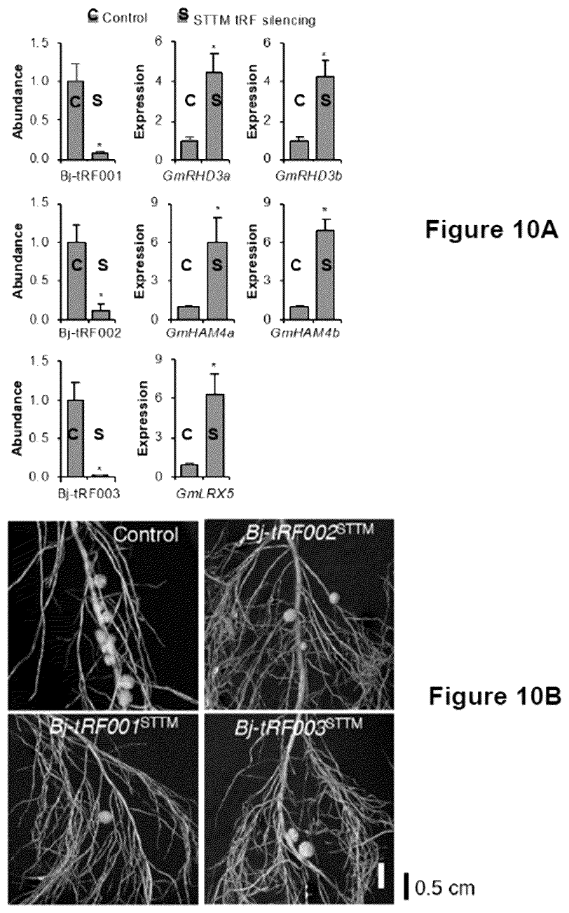

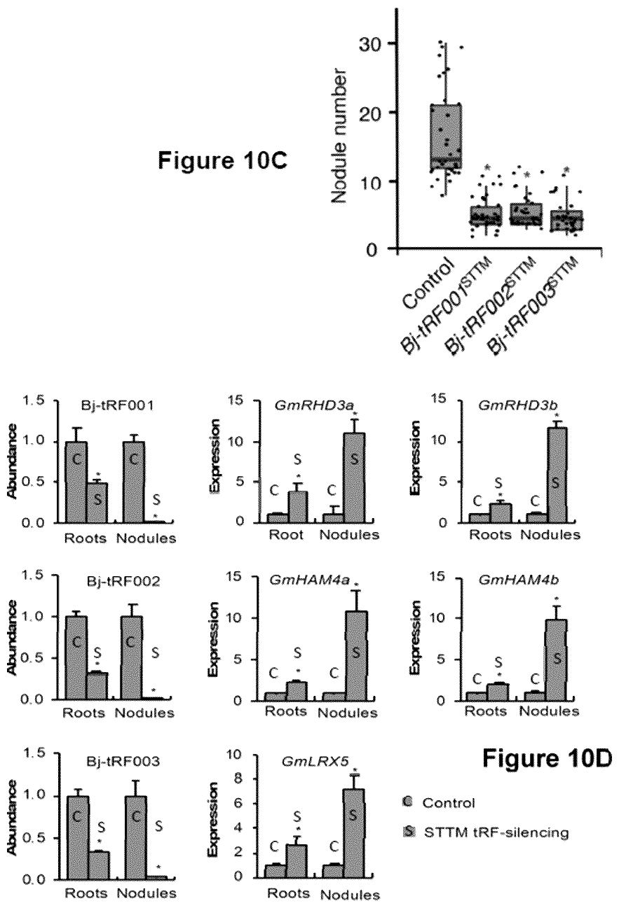

To examine the direct effects of individual rhizobial tRFs on nodulation through the cleavage of the transcripts from their targets, transgenic short tandem target mimic (STTM) soybean roots were generated to silence each of the three rhizobial tRFs. As shown in FIG. 10A, the expression of each of the three tRFs was almost completely blocked following completion of the STTM protocol described herein; consequently, the expression levels of the identified genes were significantly increased. FIG. 10D shows the reduced abundance of rhizobial tRFs and increased expression of soybean genes in the STTM hairy roots with the nodules removed and in the nodules (controls (C) and STTM roots/nodules (S)).

As shown in FIGS. 10B and 10C, nodule numbers in the STTM roots were significantly decreased as compared to those of the empty-vector transgenic controls. As expected, relative abundance of the three tRFs was also decreased and expression of the identified targets was increased, supporting that these tRFs are positive regulators of nodulation and function through repressing the associated target genes. Given the lack of sequence complementarity of these rhizobial tRFs to the transcripts of any other soybean genes in the entire genome, there is a significantly high likelihood that the effect of the tRFs on nodule formation directly results from the repressed expression of their targets.

### Example 6

Rhizobial tRFs and the Target Genes in the Host Cells Interact During Rhizobial Infection

As rhizobial infection is a critical step for initiating nodules, how tRF-mediated regulation of the five soybean target genes affects rhizobial infection was examined. The expression patterns of the five tRF targets in soybean root hairs at early stage post inoculation with USDA110 was then explored.

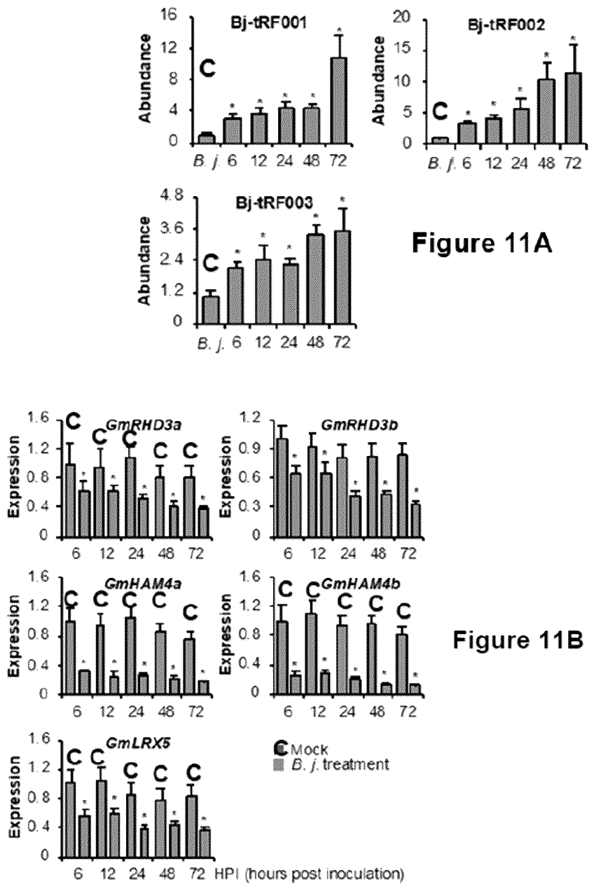

At all five time points examined, the abundance of the three tRFs targeting these genes was increased from 6 hpi to 72 hpi (FIG. 11A). By contrast, all five genes showed significantly reduced levels of expression in inoculated root hairs as compared with uninoculated root hairs (FIG. 11B). All five genes also showed decreased expression from 6 hpi to 72 hpi. This data supports that interactions do occur between rhizobial tRFs and their target genes in the host cells during rhizobial infection.

### Example 7

Root Phenotypes in Relation with Target Gene Expression and Silencing by Rhizobial tRFs

Following the profiling of host gene expression in inoculated root hairs at early stages of rhizobial infection, the morphology of inoculated root hairs in which GmRHD3b, GmHAM4a, and GmLRX5 were overexpressed by the 35S promoter were examined, as well as the morphology of inoculated root hairs in which each of the three rhizobial tRFs targeting GmRHD3a/GmRHD3b, GmHAM4a/GmHAM4b, and GmLRX5 were silenced by STTM (both studies using vector-transgenic roots as controls). For each hairy root, 1-cm segments in the mature zone were cut, fixed and examined under microscope.

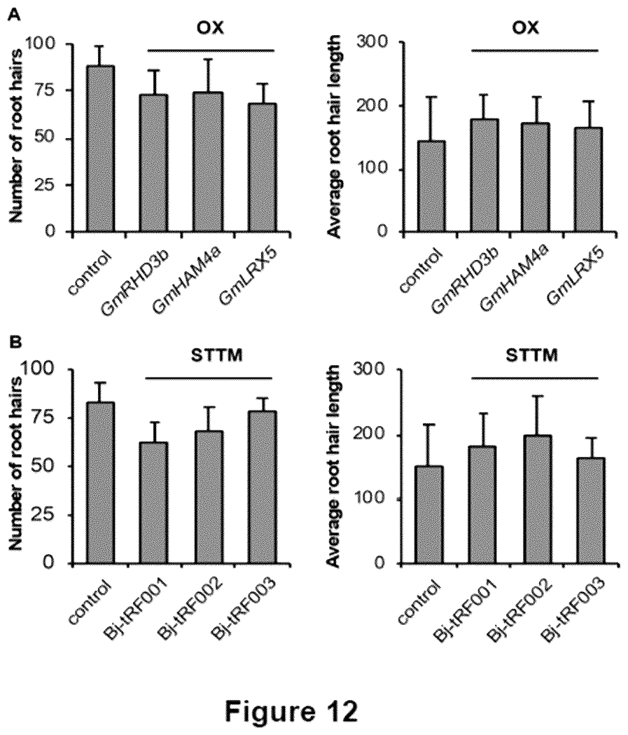

No significant differences in root hair number and root hair length were observed between each of the three GmRHD3b, GmHAM4a, and GmLRX5 overexpression roots and the controls, or between the tRF-silencing STTM roots and the controls (FIG. 12); however, the proportions of deformed and curled root hairs were significantly decreased in each of the three overexpression and STTM roots as compared with respective controls (FIG. 13, subparts A-C). It is documented that rhizobial Nod factor perception in legume roots induces expression of nodulation genes and causes curling of root hairs that entrap the Rhizobia to form nodules. In view of this, the presently described data supports that both root hair curling and nodule number are positively regulated by rhizobial tRF and that rhizobial tRFs promote rhizobial infection.

### Example 8

### Verification of Rhizobial tRF Mechanism

To better understand the mechanism through which rhizobial tRFs regulate nodulation and to confirm expression of certain constructs can result in production of the desired “artificial tRFs” in a plant in vivo, two artificial miRNA precursors, aMIR-tRF001 and aMIR-tRF003, were constructed by replacing the miR172a and miR172a* sequences from the soybean miR172a precursor MIR172a with rhizobial tRF001 and its complementary tRF001* or with tRF003 and its complementary tRF003*. FIG. 14D illustrates the structure of such artificial miRNA genes. aMIR-tRF001 and aMIR-tRF003 (identical/complementary to Bj-tRF001 and Bj-tRF003) were expressed separately in Williams 82 hairy roots under the control of 35S promoter to produce artificial miRNAs amiR-tFR001 and amiR-tFR003 in the transgenic roots (see FIG. 14A, control data labeled C, transgenic root data labeled X).

As shown in FIG. 14B, expressions of the amiR-tFR001 and amiR-tFR003 targets GmRHD3a 3b and GmLRX5 (data labeled X) was significantly reduced as compared with that of empty-vector transgenic controls (data labeled C). Likewise, as seen in FIG. 14C, more nodules were produced in the aMIR-tRF001 and aMIR-tRF003 transgenic roots (data labeled X) than in the respective controls (data labeled C). These results support that the artificial miRNA/tRF sequences did result in production of the desired artificial tRFs in the roots and directly repressed the identified host gene targets to promote nodulation.

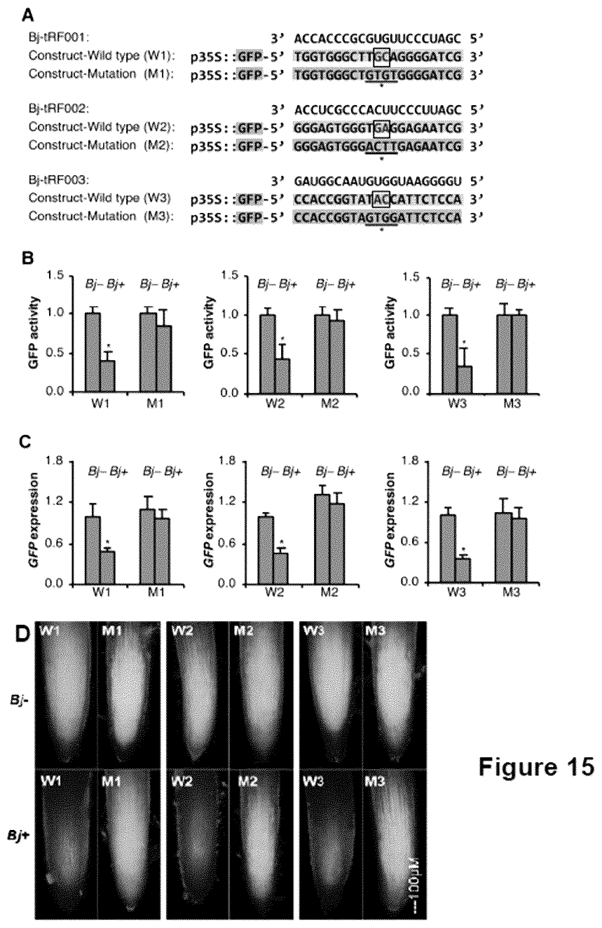

To determine the extent sequence complementarity is required for artificial miRNA/tRF-mediated gene regulation, two sets of fusion genes were made by adding each of the 21-base pair (bp) of DNA fragments corresponding to each the three identified tRF target sites (wild type) and each of the 21-bp of DNA fragments with 4-bp modification at the detected cleavage site of the targeted plant gene (mutation type) to the coding sequence of the Green Fluorescence Protein (GFP) gene. The fusion genes were then separately expressed under the control of the 35S promoter in Williams 82 hairy roots (see FIG. 15, subpart A).

As shown in FIG. 15, subparts B and D, there was a notable reduction of GFP activity in the “wild-type” roots (W1-W3) about 24 hours after inoculation with USDA110, whereas no observable change of the GFP activity in the “mutation type” roots (M1-M3). The relative abundance of GFP transcripts was also consistent with GFP activity (see FIG. 15, subpart C). The mutation type plant genes were not cleaved by the tRFs, which strongly supports the tRF cleavage mechanism described herein. Accordingly, these results indicate that the “wild-type” fusion genes were negatively regulated through base-pairing of their mRNAs at the integrated “target sites” with the rhizobial tRFs.

### Example 9

tRFs Action Through AGO1 Pathways

In Arabidopsis, AGO1 is a component of the RNA-induced silencing complexes that mediate miRNA-guided cleavage of target mRNAs. The expression of AGO1 is upregulated in response to infection by several plant viruses and has been shown to be responsible for translational inhibition or cleavage of complementary target mRNAs in the miRNA pathway.

To determine whether the rhizobial tRFs act through the functional counterpart of AGO1 in soybean, one (GmAGO1b) of the two soybean orthologs of the Arabidopsis AGO1, whose transcripts are relatively more abundant than those of the other (GmAGO1a) in soybean root nodules, was fused with the Myc epitope tag and expressed in the hairy roots of Williams 82. The fusion protein was then immunoprecipitated by the Myc antibody from the 20-day nodules induced by USDA110.

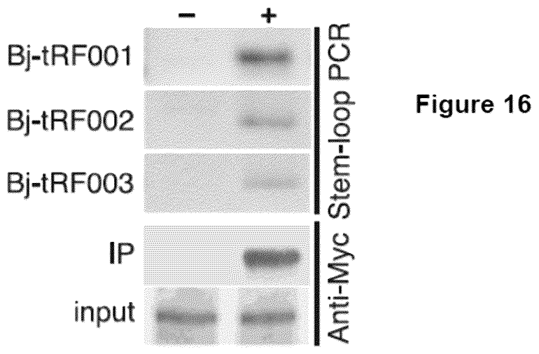

As shown in FIG. 16, all three rhizobial tRFs were detected in the GmAGO1b-Myc-associated fraction pulled down by the Myc antibody, but not detected in the nodule lysate incubated without the antibody. This data supports that these rhizobial tRFs hijacked the soybean AGO1 to instead catalyze tRF-guided cleavage of target mRNAs in the host cells.

Interestingly, referring back to Example 6, the tRF-mediated regulation of host gene expression was actually detected at early stages of rhizobial infection. At all five time points from 6 to 72 hours after inoculation with USDA110, the abundance of the three tRFs was increased in the inoculated root hairs as compared to the uninoculated root hairs (FIG. 11A), whereas the expression of their targets was decreased (FIG. 11B). This data supports tRFs do, in fact, employ an miRNA-like mechanism to regulate gene expression.

### Example 10

Sequence Conservation of Three Rhizobial tRFs, tRF Target Sites, and their Corresponding Sequences Among Different Legume-Rhizobial Partners

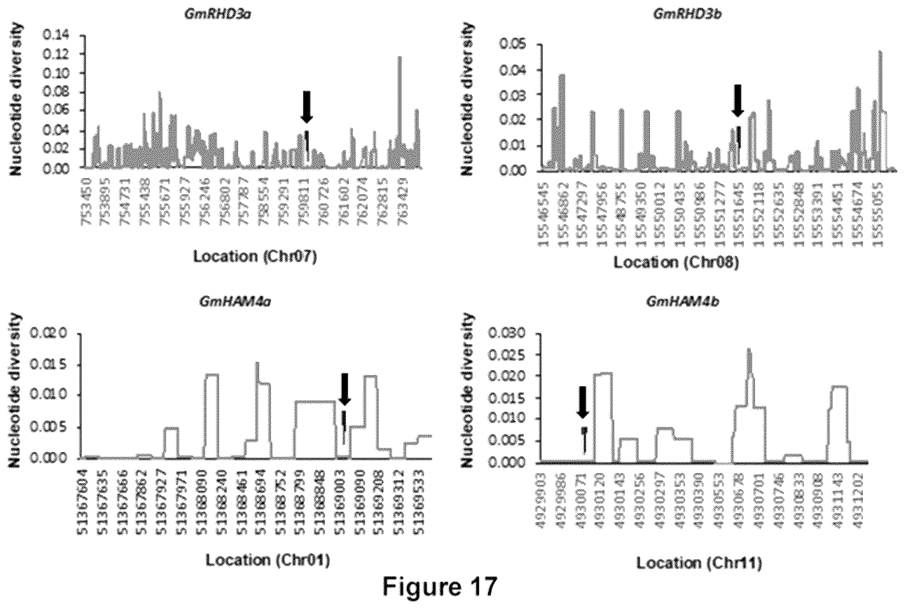

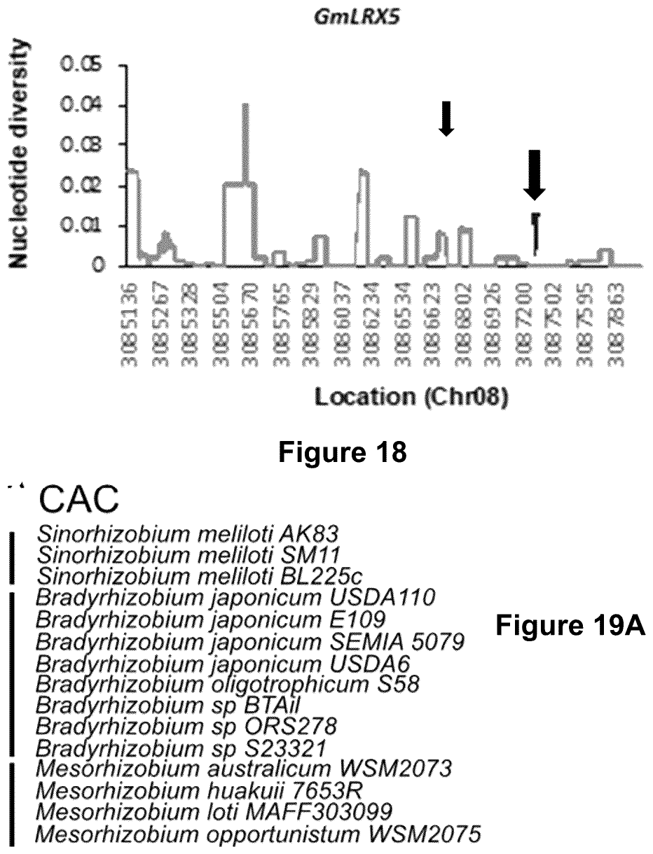

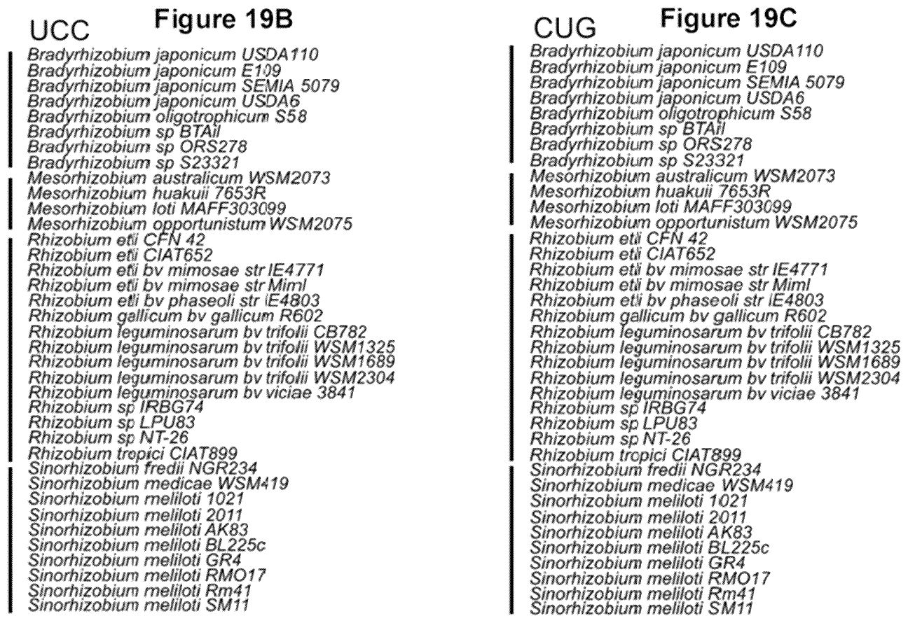

To determine if rhizobial tRF-mediated host gene regulation is conserved across evolutionarily related legumes, sequence data from four legumes (soybean, common bean (Phaseolus vulgaris), Medicago truncatula, and Lotus japonica) and 12 rhizobium species was analyzed, as well as the GmRHD3a/GmRHD3b, GmHAM4a/GmHAM4b, and GmLRX5 sequences from soybean populations, with respect to compatible symbiotic interactions. As shown in FIGS. 17 and 18, among 699 soybean accessions, no sequence variation at the three tRF target sites across the five genes was found. Further, among the eight B. japonicum strains, no sequence variation at the three tRF sites within respective rhizobial tRNAs was detected. FIG. 19A-19C lists bacterial strains/species that conserve the three tRF target sites CAC, UCC, and CUG, with the thick vertical black bars indicating different rhizobium groups.

By contrast, sequences at the target sites diverged among the four legumes (data not shown). Sinorhizobium meliloti (Sm), Mesorhizobium loti (Ml), Bradyrhizobium japonicum (Bj) and Rhizobium etli (Re), are compatible rhizobium species often used to inoculate Medicago truncatula (Mtru), Lotus japonicus (Ljap), Glycine max (Gmax) and Phaseolus vulgaris (Pvul), respectively, in scientific research. Rhizobial DNA sequences (dubbed tRF orthologs) corresponding to Bj-tRF001, Bj-tRF002, and Bj-tRF003 from four rhizobium species and the sequences (target site orthologs) corresponding to the three tRF target sites from four legume species were assessed. The highest levels of sequence complementarities between any of the tRF orthologs and any of the target site orthologs were identified, with only the sequence complementarities of (1) Bj-tRF001 and Gmax & Pvul, (2) Bj-tRF002 and Gmax, and (3) Bj-tRF003 and Gmax predicted by psRNA Target to be sufficient for miRNA-guided cleavage. In particular, no orthologs of GmLRX5 were found in the other three legumes. The counterparts of the three rhizobial tRF sequences in respective tRNAs also showed interspecific divergence (data not shown).

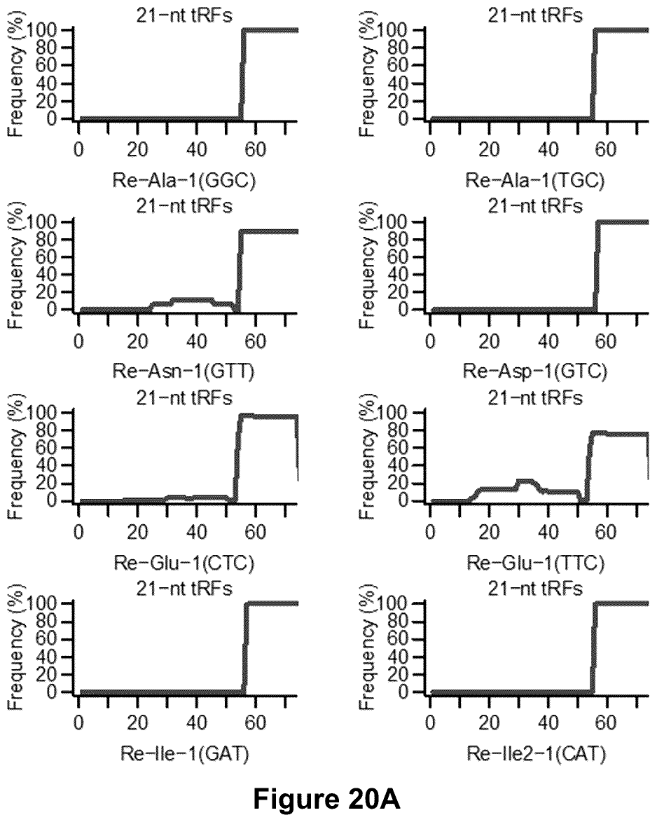

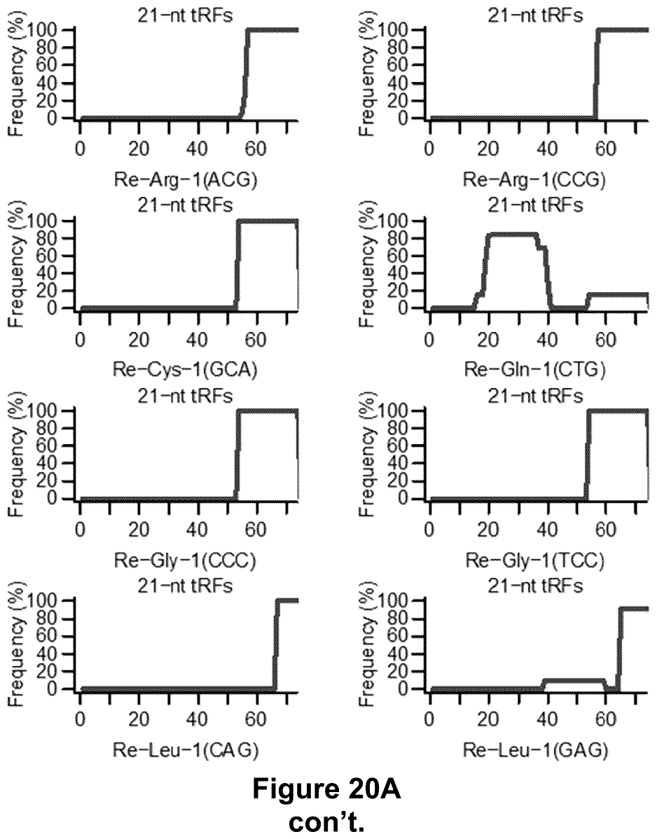

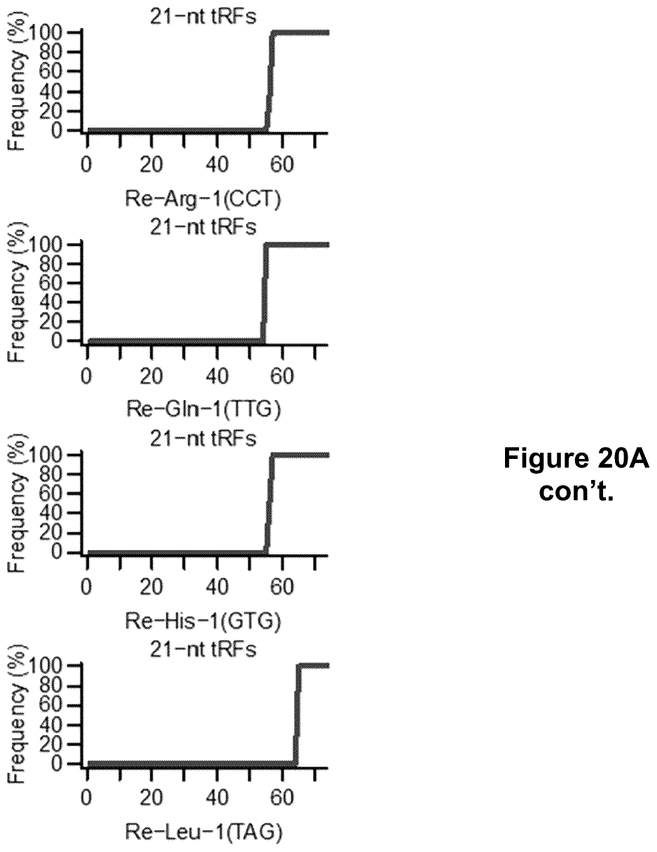

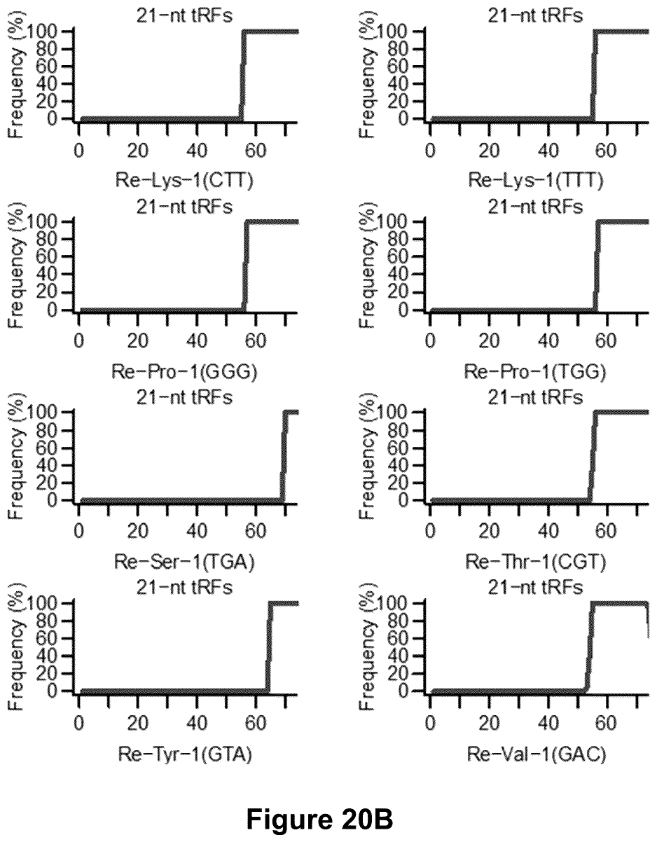

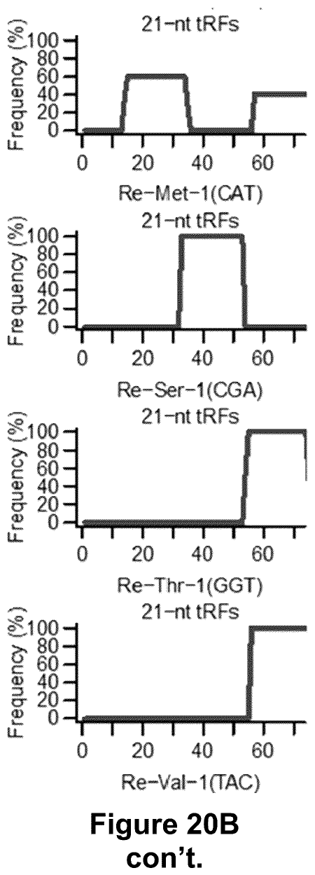

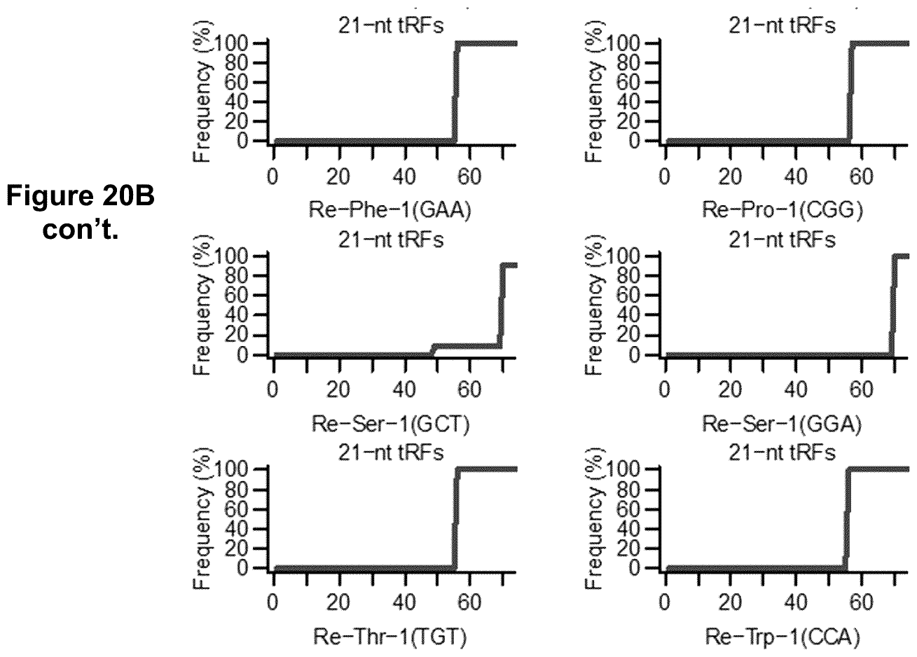

However, while PvRHD3 in common bean, the ortholog of GmRHD3a/3b, does have a tRF001 target site identical to that of GmRHD3a/3b, Rhizobium etli, a compatible symbiotic partner of common bean, does not have the B. japonicum Val-1-tRNA (CAC) from which tRF001 was derived. Using the small RNA data from the common bean nodules induced by a R. etli strain, 38 R. etli tRNAs were identified to have produced 21-nt tRFs. As shown in FIGS. 20A and 20B, these tRFs were primarily derived from the 3′ ends of the tRNAs.

Ten different 21-nt tRFs, each with a relative abundance of >100 counts per million rhizobial small RNA reads in the common bean nodules, were predicted to target 14 common bean genes, including genes encoding a protein kinase, a GRAS transcription factor, and an APETALA2-like transcription factor that is thought to be involved in nodulation regulation (see Table 4).

TABLE 4
Rhizobial tRFs from Rhizobium etli and their potential targets in common bean.

| tRF-seq | tRNA | Gene Symbol | Annotation |
|---|---|---|---|
| SEQ ID NO: 34 | Met-CAT | AtPERK12, IGI1 | Protein kinase superfamily protein |
| SEQ ID NO: 35 | Ile-GAT | | P-loop containing nucleoside triphosphate |
| | | | hydrolases superfamily protein |
| SEQ ID NO: 36 | Gln-CTG | HSP70T-2 | heat-shock protein 70T-2 |
| SEQ ID NO: 37 | Met-CAT | BLH1, EDA29 | BEL1-like homeodomain 1 |
| SEQ ID NO: 38 | Met-CAT | ABCA1, | ATP-binding cassette A1 |
| | | AtABCA1 | |
| SEQ ID NO: 39 | Met-CAT | | Ubiquitin-like superfamily protein |
| SEQ ID NO: 40 | Ile-GAT | ATXIA, XIA | myosin XI A |
| SEQ ID NO: 41 | Pro-CGG | RGA, RGA1 | GRAS family transcription factor family |
| | | | protein |
| SEQ ID NO: 42 | Asn-GTT | | Pyridoxal-dependent decarboxylase family |
| | | | protein |
| SEQ ID NO: 43 | Trp-CCA | HEMA1 | Glutamyl-tRNA reductase family protein |
| SEQ ID NO: 44 | Val-TAC | AP2, FL1, FLO2 | Integrase-type DNA-binding superfamily |
| | | | protein |
| SEQ ID NO: 45 | Glu-TTC | | Zinc finger, C3HC4 type (RING finger) family |
| | | | protein |
| SEQ ID NO: 46 | Glu-TTC | ACL5 | S-adenosyl-L-methionine-dependent |
| | | | methyltransferases superfamily protein |
| SEQ ID NO: 47 | Met-CAT | KAN, KAN1 | Homeodomain-like superfamily protein |
| SEQ ID NO: 48 | Met-CAT | BLH1, EDA29 | BEL1-like homeodomain 1 |
| SEQ ID NO: 49 | Met-CAT | | Ubiquitin-like superfamily protein |

Nevertheless, none of these 14 putative R. etli tRF targets in common bean are orthologs of the 25 targeted B. japonicum tRF targets in soybean (see Table 2).

The results support that, while the use of bacterial tRFs to target host genes appears to be conserved, the tRFs and their target genes may vary across species. Indeed, based on the data presented herein, the rhizobial-derived sequences and paired R. elti tRF gene targets of Table 3 above can be leveraged in the same fashion as described in connection with the B. japonicum tRFs/gene targets described herein. These findings, combined with the other studies presented herein, highlight the complexity of the regulatory network that occurs during the establishment of nodulation.

Accordingly, the data presented herein demonstrates that rhizobial tRFs are positive regulators of rhizobial infection and nodule formation in soybean and play an important role in balancing plant growth and symbiosis. In addition to the rhizobial tRFs identified herein, the other rhizobial tRFs identified in Table 2 are predicted to target soybean genes annotated to encode auxin receptors and efflux carriers, RING/U-box proteins, and protein kinases, which likely affects nodulation. Furthermore, the data supports that such cross-kingdom communications are likely common among symbiotic partners, but the nodes of rhizobial tRFs-host gene interactions appear to be diverse. These findings have exemplified the effectiveness of genome editing for enhancement of the nodulation capability of leguminous plants.

While various embodiments of compounds, transgenic plants/plant roots/plant cells, and methods hereof have been described in considerable detail, the embodiments are merely offered by way of non-limiting examples. Many variations and modifications of the embodiments described herein will be apparent to one of ordinary skill in the art in light of the disclosure. It will therefore be understood by those skilled in the art that various changes and modifications may be made, and equivalents may be substituted for elements thereof, without departing from the scope of the disclosure. Indeed, this disclosure is not intended to be exhaustive or too limiting. The scope of the disclosure is to be defined by the appended claims, and by their equivalents.

Further, in describing representative embodiments, the disclosure may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

1-27. (canceled)

28. A construct encoding one or more ribonucleic acid (RNA) fragments that fold into a hairpin structure and are cleaved in planta to result in one or more artificial transfer RNA-derived fragments (tRFs),
wherein when the construct is expressed in a leguminous plant or a leguminous plant root, increases root nodule formation when the leguminous plant or leguminous plant root is inoculated with a Bradyrhizobium or Sinorhizobium species or strain as compared to a corresponding leguminous plant or leguminous plant root that does not comprise the at least one small RNA fragment.

29. The construct of claim 28, wherein each of the at least one small RNA fragments has a nucleotide sequence selected from a group consisting of SEQ ID NOs. 1-3 and 50-70.

30. The construct of claim 28, wherein the leguminous plant or leguminous plant root is a Glycine max plant or plant root.

31. The construct of claim 28, wherein at least one of the one or more RNA fragments downregulates at least one target gene of the leguminous plant or leguminous plant root that is a negative regulator of nodulation.

32. The construct of claim 31, wherein the target gene is:
GmRHD3a which encodes the amino acid sequence of SEQ ID NO: 80,
GmRHD3b which encodes the amino acid sequence of SEQ ID NO: 81,
GmHAM4a which encodes the amino acid sequence of SEQ ID NO: 82,
GmHAM4b which encodes the amino acid sequence of SEQ ID NO: 83, or
GmLRX5 which encodes the amino acid sequence of SEQ ID NO: 84.

33. The construct of claim 31, wherein the at least one target gene comprises:
GmRHD3a which encodes the amino acid sequence of SEQ ID NO: 80 and GmRHD3b which encodes the amino acid sequence of SEQ ID NO: 81, or
GmHAM4a which encodes the amino acid sequence of SEQ ID NO: 83 and GmHAM4b which encodes the amino acid sequence of SEQ ID NO: 83.

34. The construct of claim 28, wherein the each of the one or more artificial tRFs has a nucleotide sequence selected from the group consisting of SEQ ID NOS: 34-49.

35. A method for increasing nodulation activity in a Glycine max plant, plant root, or plant cell, the method comprising introducing or expressing the construct of claim 1 into the Glycine max plant, plant root, or plant cell; and
inoculating, or having inoculated, the modified Glycine max plant, plant root, or plant cell with at least one Bradyrhizobium or Sinorhizobium species or strain.

36. The method of claim 35, wherein at least one of the one or more RNA fragments downregulates at least one target gene of the Glycine max plant, plant root, or plant cell that is a negative regulator of nodulation.

37. The method of claim 36, wherein the at least one target gene is selected from the group consisting of GmRHD3a which encodes the amino acid sequence of SEQ ID NO: 80, GmRHD3b which encodes the amino acid sequence of SEQ ID NO: 81, GmHAM4a which encodes the amino acid sequence of SEQ ID NO: 82, GmHAM4b which encodes the amino acid sequence of SEQ ID NO: 83, and GmLRX5 which encodes the amino acid sequence of SEQ ID NO: 84.

38. The method of claim 36, further comprising expressing a CRISPR-Cas9 vector in the Glycine max plant, plant root, or plant cell that is constructed to silence the at least one target gene.


SEQUENCE LISTING (84 sequences)

SEQ ID NO: 1 - RNA, 21 nt, Bradyrhizobium japonicum
  source 1..21: mol_type = genomic RNA
cgatcccttgtgcgcccacca

SEQ ID NO: 2 - RNA, 21 nt, Bradyrhizobium japonicum
  source 1..21: mol_type = genomic RNA
cgattcccttcacccgctcca

SEQ ID NO: 3 - RNA, 21 nt, Bradyrhizobium japonicum
  source 1..21: mol_type = genomic RNA
tggggaatggtgtaacggtag

SEQ ID NO: 4 - DNA, 23 bp, synthetic construct
  source 1..23: mol_type = other DNA; note = oligonucleotides for targeting a RHD3 gene (RHD3_pSC_F)
attgaggtgaagttggctgaatg

SEQ ID NO: 5 - DNA, 23 bp, synthetic construct
  source 1..23: mol_type = other DNA; note = oligonucleotides for targeting a RHD3 gene (RHD3_pSC_B)
aaccattcagccaacttcacctc

SEQ ID NO: 6 - DNA, 23 bp, synthetic construct
  source 1..23: mol_type = other DNA; note = oligonucleotides for targeting a HAM4 gene (HAM4_pSC_F)
attgcctgtggggatgagggaac

SEQ ID NO: 7 - DNA, 23 bp, synthetic construct
  source 1..23: mol_type = other DNA; note = oligonucleotides for targeting a HAM4 gene (HAM4_pSC_B)
aacgttccctcatccccacaggc

SEQ ID NO: 8 - DNA, 23 bp, synthetic construct
  source 1..23: mol_type = other DNA; note = oligonucleotides for targeting a LRX5 gene (LRX5_pSC-F)
attgttgaaactgctctacgaac

SEQ ID NO: 9 - DNA, 23 bp, synthetic construct
  source 1..23: mol_type = other DNA; note = oligonucleotides for targeting a LRX5 gene (LRX5_pSC-B)
aacgttcgtagagcagtttcaac

SEQ ID NO: 10 - DNA, 21 bp, Bradyrhizobium japonicum
  source 1..21: mol_type = genomic DNA
caaatcctatccccgcaacca

SEQ ID NO: 11 - DNA, 21 bp, Bradyrhizobium japonicum
  source 1..21: mol_type = genomic DNA
caaatcctctcgctccgacca

SEQ ID NO: 12 - DNA, 21 bp, Bradyrhizobium japonicum
  source 1..21: mol_type = genomic DNA
caatcctgtctggcagcacca

SEQ ID NO: 13 - DNA, 21 bp, Bradyrhizobium japonicum
  source 1..21: mol_type = genomic DNA
cgaatccagctgccccgacca

SEQ ID NO: 14 - DNA, 21 bp, Bradyrhizobium japonicum
  source 1..21: mol_type = genomic DNA
cgaatccaggttccccagcca

SEQ ID NO: 15 - DNA, 21 bp, Bradyrhizobium japonicum
  source 1..21: mol_type = genomic DNA
cgaatcccactctctccgcca

SEQ ID NO: 16 - DNA, 21 bp, Bradyrhizobium japonicum
  source 1..21: mol_type = genomic DNA
cgaatccctccctctccgcca

SEQ ID NO: 17 - DNA, 21 bp, Bradyrhizobium japonicum
  source 1..21: mol_type = genomic DNA
cgaatcctgccactccgacca

SEQ ID NO: 18 - DNA, 21 bp, Bradyrhizobium japonicum
  source 1..21: mol_type = genomic DNA
cgaatcgtgtcgggtgcgcca

SEQ ID NO: 19 - DNA, 21 bp, Bradyrhizobium japonicum
  source 1..21: mol_type = genomic DNA
cgagcccacccaactgtacca

SEQ ID NO: 20 - DNA, 21 bp, Bradyrhizobium japonicum
  source 1..21: mol_type = genomic DNA
cgagccctgccactcctgcca

SEQ ID NO: 21 - DNA, 21 bp, Bradyrhizobium japonicum
  source 1..21: mol_type = genomic DNA
cgagtcatcccgggatcgcca

SEQ ID NO: 22 - DNA, 21 bp, Bradyrhizobium japonicum
  source 1..21: mol_type = genomic DNA
cgagtccctccgagcgcacca

SEQ ID NO: 23 - DNA, 21 bp, Bradyrhizobium japonicum
  source 1..21: mol_type = genomic DNA
cgatcccgtctggctccacca

SEQ ID NO: 24 - DNA, 21 bp, Bradyrhizobium japonicum
  source 1..21: mol_type = genomic DNA
cgatccctgtcgtgcccacca

SEQ ID NO: 25 - DNA, 21 bp, Bradyrhizobium japonicum
  source 1..21: mol_type = genomic DNA
cgatcccttgtgcgcccacca

SEQ ID NO: 26 - DNA, 21 bp, Bradyrhizobium japonicum
  source 1..21: mol_type = genomic DNA
cgattcccttcacccgctcca

SEQ ID NO: 27 - DNA, 21 bp, Bradyrhizobium japonicum
  source 1..21: mol_type = genomic DNA
cgattcccttcgcccgctcca

SEQ ID NO: 28 - DNA, 21 bp, Bradyrhizobium japonicum
  source 1..21: mol_type = genomic DNA
cgattccgcccctgggcacca

SEQ ID NO: 29 - DNA, 21 bp, Bradyrhizobium japonicum
  source 1..21: mol_type = genomic DNA
gcgctcgtggcggaactggta

SEQ ID NO: 30 - DNA, 21 bp, Bradyrhizobium japonicum
  source 1..21: mol_type = genomic DNA
gcgggtgtagctcagtggtag

SEQ ID NO: 31 - DNA, 21 bp, Bradyrhizobium japonicum
  source 1..21: mol_type = genomic DNA
tcctcggtagctcagcggtag

SEQ ID NO: 32 - DNA, 21 bp, Bradyrhizobium japonicum
  source 1..21: mol_type = genomic DNA
tggggaatggtgtaacggtag

SEQ ID NO: 33 - DNA, 21 bp, Bradyrhizobium japonicum
  source 1..21: mol_type = genomic DNA
ttggtagacgcaagggactta

SEQ ID NO: 34 - DNA, 21 bp, Rhizobium etli
  source 1..21: mol_type = genomic DNA
caaatccctcctccgctacca

SEQ ID NO: 35 - DNA, 21 bp, Rhizobium etli
  source 1..21: mol_type = genomic DNA
caagtcttcccgggcccacca

SEQ ID NO: 36 - DNA, 21 bp, Rhizobium etli
  source 1..21: mol_type = genomic DNA
cgagtccaggttccccagcca

SEQ ID NO: 37 - DNA, 21 bp, Rhizobium etli
  source 1..21: mol_type = genomic DNA
agtaggtcagagcagaggaat

SEQ ID NO: 38 - DNA, 21 bp, Rhizobium etli
  source 1..21: mol_type = genomic DNA
agtaggtcagagcagaggaat

SEQ ID NO: 39 - DNA, 21 bp, Rhizobium etli
  source 1..21: mol_type = genomic DNA
agtaggtcagagcagaggaat

SEQ ID NO: 40 - DNA, 21 bp, Rhizobium etli
  source 1..21: mol_type = genomic DNA
caagtcttcccgggcccacca

SEQ ID NO: 41 - DNA, 21 bp, Rhizobium etli
  source 1..21: mol_type = genomic DNA
cgaatcactccactccgacca

SEQ ID NO: 42 - DNA, 21 bp, Rhizobium etli
  source 1..21: mol_type = genomic DNA
cgaatccggcccggggagcca

SEQ ID NO: 43 - DNA, 21 bp, Rhizobium etli
  source 1..21: mol_type = genomic DNA
cgagccctgctgcccctgcca

SEQ ID NO: 44 - DNA, 21 bp, Rhizobium etli
  source 1..21: mol_type = genomic DNA
cgagtctctcatcgcccacca

SEQ ID NO: 45 - DNA, 21 bp, Rhizobium etli
  source 1..21: mol_type = genomic DNA
gattttcattctgtaaagagg

SEQ ID NO: 46 - DNA, 21 bp, Rhizobium etli
  source 1..21: mol_type = genomic DNA
gattttcattctgtaaagagg

SEQ ID NO: 47 - DNA, 21 bp, Rhizobium etli
  source 1..21: mol_type = genomic DNA
gtaggtcagagcagaggaatc

SEQ ID NO: 48 - DNA, 21 bp, Rhizobium etli
  source 1..21: mol_type = genomic DNA
gtaggtcagagcagaggaatc

SEQ ID NO: 49 - DNA, 21 bp, Rhizobium etli
  source 1..21: mol_type = genomic DNA
gtaggtcagagcagaggaatc

SEQ ID NO: 50 - RNA, 21 nt, Bradyrhizobium japonicum
  source 1..21: mol_type = genomic RNA
caaatcctatccccgcaacca

SEQ ID NO: 51 - RNA, 21 nt, Bradyrhizobium japonicum
  source 1..21: mol_type = genomic RNA
caaatcctctcgctccgacca

SEQ ID NO: 52 - RNA, 21 nt, Bradyrhizobium japonicum
  source 1..21: mol_type = genomic RNA
caatcctgtctggcagcacca

SEQ ID NO: 53 - RNA, 21 nt, Bradyrhizobium japonicum
  source 1..21: mol_type = genomic RNA
cgaatccagctgccccgacca

SEQ ID NO: 54 - RNA, 21 nt, Bradyrhizobium japonicum
  source 1..21: mol_type = genomic RNA
cgaatccaggttccccagcca

SEQ ID NO: 55 - RNA, 21 nt, Bradyrhizobium japonicum
  source 1..21: mol_type = genomic RNA
cgaatcccactctctccgcca

SEQ ID NO: 56 - RNA, 21 nt, Bradyrhizobium japonicum
  source 1..21: mol_type = genomic RNA
cgaatccctccctctccgcca

SEQ ID NO: 57 - RNA, 21 nt, Bradyrhizobium japonicum
  source 1..21: mol_type = genomic RNA
cgaatcctgccactccgacca

SEQ ID NO: 58 - RNA, 21 nt, Bradyrhizobium japonicum
  source 1..21: mol_type = genomic RNA
cgaatcgtgtcgggtgcgcca

SEQ ID NO: 59 - RNA, 21 nt, Bradyrhizobium japonicum
  source 1..21: mol_type = genomic RNA
cgagcccacccaactgtacca

SEQ ID NO: 60 - RNA, 21 nt, Bradyrhizobium japonicum
  source 1..21: mol_type = genomic RNA
cgagccctgccactcctgcca

SEQ ID NO: 61 - RNA, 21 nt, Bradyrhizobium japonicum
  source 1..21: mol_type = genomic RNA
cgagtcatcccgggatcgcca

SEQ ID NO: 62 - RNA, 21 nt, Bradyrhizobium japonicum
  source 1..21: mol_type = genomic RNA
cgagtccctccgagcgcacca

SEQ ID NO: 63 - RNA, 21 nt, Bradyrhizobium japonicum
  source 1..21: mol_type = genomic RNA
cgatcccgtctggctccacca

SEQ ID NO: 64 - RNA, 21 nt, Bradyrhizobium japonicum
  source 1..21: mol_type = genomic RNA
cgatccctgtcgtgcccacca

SEQ ID NO: 65 - RNA, 21 nt, Bradyrhizobium japonicum
  source 1..21: mol_type = genomic RNA
cgattcccttcgcccgctcca

SEQ ID NO: 66 - RNA, 21 nt, Bradyrhizobium japonicum
  source 1..21: mol_type = genomic RNA
cgattccgcccctgggcacca

SEQ ID NO: 67 - RNA, 21 nt, Bradyrhizobium japonicum
  source 1..21: mol_type = genomic RNA
gcgctcgtggcggaactggta

SEQ ID NO: 68 - RNA, 21 nt, Bradyrhizobium japonicum
  source 1..21: mol_type = genomic RNA
gcgggtgtagctcagtggtag

SEQ ID NO: 69 - RNA, 21 nt, Bradyrhizobium japonicum
  source 1..21: mol_type = genomic RNA
tcctcggtagctcagcggtag

SEQ ID NO: 70 - RNA, 21 nt, Bradyrhizobium japonicum
  source 1..21: mol_type = genomic RNA
ttggtagacgcaagggactta

SEQ ID NO: 71 - RNA, 75 nt, Bradyrhizobium japonicum
  source 1..75: mol_type = genomic RNA
gggcgcatagctcagcgggagagcgttcccttcacacgggagaggtccaaggttcgatcccttgtgcgcccacca

SEQ ID NO: 72 - RNA, 74 nt, Bradyrhizobium japonicum
  source 1..74: mol_type = genomic RNA
gcgggtgtagctcaatggtagagcagcagccttccaagctgaatacgagggttcgattcccttcacccgctcca

SEQ ID NO: 73 - RNA, 74 nt, Bradyrhizobium japonicum
  source 1..74: mol_type = genomic RNA
tggggaatggtgtaacggtagcacaacagactctgactctgtttgtcttggttcgaatccaggttccccagcca

SEQ ID NO: 74 - DNA, 33 bp, synthetic construct
  source 1..33: mol_type = other DNA; note = a forward primer for an overexpression construct for target gene RHD3b
gcgcgtcgacatggcaaatagtgagacttgttg

SEQ ID NO: 75 - DNA, 34 bp, synthetic construct
  source 1..34: mol_type = other DNA; note = a reverse primer for an overexpression construct for target gene RHD3b
gcgctctagactactcatcttttaatggacttgc

SEQ ID NO: 76 - DNA, 34 bp, synthetic construct
  source 1..34: mol_type = other DNA; note = a forward primer for an overexpression construct for target gene HAM4a
ttgcctcgagatgagagttcccgttccctcatcc

SEQ ID NO: 77 - DNA, 35 bp, synthetic construct
  source 1..35: mol_type = other DNA; note = a reverse primer for an overexpression construct for target gene HAM4a
gcttggtaccctaacagcgccatgctgacgtggca

SEQ ID NO: 78 - DNA, 35 bp, synthetic construct
  source 1..35: mol_type = other DNA; note = a forward primer for an overexpression construct for target gene LRX5
gcgcgtcgacatgatgatgatgatgaagaagaagg

SEQ ID NO: 79 - DNA, 35 bp, synthetic construct
  source 1..35: mol_type = other DNA; note = a reverse primer for an overexpression construct for target gene LRX5
gcaatctagactaataaaagggtggtggtggaggc

SEQ ID NO: 80 - protein, 808 aa, Glycine max
MANSETCCSTQLIDGDGTFNVFGVENFMKEVKLAECGLSYAVVSIMGPQSSGKSTLLNHLFGTNFREMDAFKGRSQTTKGIWMARCGIEPCTLVMDLEGTDGRERGEDDTAFEKQSALFALAVSDIVLINMWCHDIGREQAANKPLLKTVFQVMMRLFSPRKTTLLFVIRDKTRTPLENLEPVLREDIQKIWDSVPKPQAHKETPLSEFFNVEVVALSSYEEKEEQFKDQVASLRQRFHHSIAPGGLAGDRRGVVPASGFSFSSEHIWKVIKENKDLDLPAHKVMVATVRCEEIANEKYVASFVANEDWCQLEEAVQSGPIPGFGKKLSSLLDICFSEYDAEATYFDEGVRSSKQKQLQEKLFQLVQPAFQSALGHIRSGTLDKFKEAFDKTLKGGEGFSVAANNCIGSCMVQFDEACTDVVIEQTNWDTSKVREKLLRDIDAHVATVRATKISELTSSYEEKLKQALSGPVEALLDGANSDTWPSIRNLFRRETESAVSGFSAALTGFDMDEETRQKIILSLEDYARGLVEGKAREEAGRVLIRMKDRFTMLFSHDSDSMPRVWTGKEDIRAITKTARSSSLKLLSVMAAIRLDDDDTDNIEKVLAVALVDSSPNSNATRSMTMVDPLASSSWEQVSSSKTLITPVQCKSLWRQFKTETEYSVSQAISAQEANKRNNNWLPPPWAIVALVILGFNEFMTLLRNPLYLGVIFVGFLLIKALWVQLDVSGEFRNGALPGIISLSSKFIPTIMNLMKKLAEEGQNPAANNPQRTPSKSSYNEGHAVSSSASSNLTRLDNGTEYASPLKDD

SEQ ID NO: 81 - protein, 808 aa, Glycine max
MANSETCCSTQLIDGDGTFNVSGLESFMKEVKLAECGLSYAVVSIMGPQSSGKSTLLNNLFGTNFREMDAFKGRSQTTKGIWMARCADIEPCTLVMDLEGTDGRERGEDDTAFEKQSALFALAVSDIVLINMWCHDIGREQAANKPLLKTVFQVMMRLFSPRKTTLLFVIRDKTRTPLENLEPVLREDIQKIWDSVPKPQAHKETPLSEFFNVEVVALSSYEEKEEQFKEQVASLQKRFHHSIAPGGLAGDRRGVVPASGFSFSSEHIWKVIKENKDLDLPAHKVMVATVRCEEIANEKYASFVANEDWCQLEEAVQSGPIPGFGKKLSSLLDTCFSEYDAEATYFDEGVRSSKQKQLQEKLFQLVQPAFQSALGHIRSGTLDKFKEAFDKALKGGEGFSVAANNCIGSGLVQFDEACTDVVIEQTNWDTSKVREKLLRDIDAYVATVRATKISELTSSYEEKLKQALSGPVEALLDGANRDTWPSIRNLLRRETESAVSGFSAALTGFDMDEETRQKMILSLEAYARGLVEGKAREEAGRVLMRMKDRFTMLFSHDSDSMPRVWTGKEDIRAITKTARSSSLKLLSVMAAIRLDDDDTDNIEKVLAVALVDSSPSSNATRSITMVDPLASSSWEQVSSSKTLITPVQCKSLWRQFKTETEYSVSQAISAQEANKRNNNWLPPPWAIVALVILGFNEFMTLLRNPLYLGVIFVGFLLIKALWVQLDVSGEFRNGALPGIISLSSKFIPTIMNLMRKLAEEGQNPAANNPQRTPSKNSYNDGHAVSSSASSNLTALDNGTEYASPLKDE

SEQ ID NO: 82 - protein, 525 aa, Glycine max
MRVPVPSSPQANPKPTNNVVRTIALPNLNNSTTPPTGLCYEPTSVLDLCRSPSPGTEKPTTDHSVLVTNSQDYLDLEDHALHNLDWDSIMKDLGLHDDSATPVLKTFLHPDDDDDDDNNNNPSCDDFTLFDHALEFTTLSDIYSNQNFAFDFNHLPHDFNHLNGFDFIEELIRAADCFDTKQLHVAQLILERLNQRLRSPVGKPLHRAAFYLKEALQSLLSGSNRTPRISSLVEIVHSIRTFKAFSGISPIPMFSIFTTNQIVLDHAASSFMHVIDFDIGLGIQYASLMKEIAEKAADSPVLRITAVVPEEYAVESTLVRDNLAQFALDLRIRVQVEFVPLRTFENLSFKAVKFVNGENTAVLLSPAIFRHLGNAAAFLADVRRISPSVVVFVDGEGWAETATASAASFRRGVVSSLEYYSMMLESLDASTVGGGGEWVRRIEMMQLRPKILAAVESAWRRVPPWREAFYGAGMRPVQLSQFADFQAECLLAKSQIRGFHVAKRQNELVLFWHDRAIVATSAWRC

SEQ ID NO: 83 - protein, 516 aa, Glycine max
MRVPVPSSPQANPKPTNNVVRTIALPNLNTTTTTTTTSLCYEPTSVLDLCRSPSPSPGTEKPTNDHAVLDLDDHALHNLDWDSIMKDLGLHDDSATPVLKTFLHPDDDNNNPSCDDFTPFDHALEFTSLSDIYSNQNLAFDFNHLPHDFNHHLNGFDFIEELIRAADCFDTKQLHVAQVILERLNQRLRSPVGKPLQRAAFYFKEALQSLLSGSNRTPRISSLVIEVHSIRTFKAFSGISPIPMFSIFTTNQIVLDHAACSFMHVIDFDIGLGIQYASLMKEIAEKAAESPVLRITAVVPEEYAVESTLVHDNLAQFALELRIRVQVEFVALRTFENLSFKSVKFVDGENTTVLLSPAIFGHLGNAAAFLADVRRISPSMVVFVDGEGWAETATASAASFRRGVVSSLEYYSMMLESLDASTVGGGGEWVRRIEMMQLGPKILAAVESAWRKLPPWREAFYGAGMRPVQLSQFADFQAECLLRKSQIRGFHVARRQNELVLFWHDRAMVATSAWRC

SEQ ID NO: 84 - protein, 735 aa, Glycine max
MMMMMKKKDAAYSSILTLLLIATLLSLTVSASSLSDAEAMYVKQRQLLYYRDEFGDRGENVIVDPSLAFENDRLRNAYIALQAWKQAILSDPRNQTADWVGSDVCSYTGVYCAPALDIPKIRTVAGIDLNHCDIAGYLPEELGLLLDLALFHVNSNRFCGTVPHKFERLKLLYELDLSNNRFAGNFPEVVLRLPSLKFLDLRFNEFEGTVPRELFDKDLDAIFINDNRFVFDLPDNFGNSPVSVIVLANNRFHGCIPASLGNMSNLNEIILMNNAFRSCLPSEIGLLKNLTVFDVSFNQLLGPIPNAVGNAVSLENLNVAHNLLSGNIPASICMLPHLQNFTYSFNFFTGEPPACLALPAFDDRVNCLPARPLQRPPAQCKSFLSKPVDCNSFKCKHFVPSPPSPSPFPSPTAPVTPSPSPSTPSPFPSPTTPVTPPSPSHSPPSPSTPSPTTPVTPSPSHSPPSPATPSPFPSPTTPVTPSPSHSPPSPATPSPFPSPTTPVTPSPSHSPPSPATPTTPVTPSPSHSPPSPATPSPFPSPHHPVTPSPSHSPPSPATPSPFPSPTTPVTPSPPSPSTPSPFPSPTTPVTPSPVHSPPSPVYLPPPHSSPTPPAHNYGSPPPHHSPAQPVYPYLSPPPPPSVHSSPPPPPPPPCIELPPPPPQSTPSPTPYLPPPSPSPPPPPPPVYHSPPPASPPPCETPPSVSPPPPPAPVYEGPLPPIVGLPYASPPPPPFY